(19) United States
(12) Patent Application Publication
(10) Pub. No.: US 2016/0137630 A1
(43) Pub. Date: May 19, 2016

(54) INDUCTION OF GATA2 BY HDAC1 AND HDAC2 INHIBITORS

(71) Applicant: ACETYLON PHARMACEUTICALS, INC., Boston, MA (US)

(72) Inventors: Jeffrey R. Shearstone, Framingham, MA (US); Matthew B. Jarpe, Quincy, MA (US)

(21) Appl. No.: 14/878,536

(22) Filed: Oct. 8, 2015

Related U.S. Application Data
(60) Provisional application No. 62/195,565, filed on Jul. 22, 2015.
(60) Provisional application No. 62/189,049, filed on Jul. 6, 2015.
(60) Provisional application No. 62/088,007, filed on Dec. 5, 2014.
(60) Provisional application No. 62/061,200, filed on Oct. 8, 2014.

(51) Int. Cl. C07D 409/14; C07D 295/155; C07D 333/24; C07D 409/12

(52) U.S. Cl. CPC C07D 409/14; C07D 409/12; C07D 295/155; C07D 333/24

(57) ABSTRACT

Provided herein are compounds, pharmaceutical compositions comprising such compounds, and methods of using such compounds to treat diseases or disorders associated with Gata2 deficiency, particularly diseases or disorders that involve any type of HDAC1 and/or HDAC2 expression. Such diseases include acute myeloid leukemia (AML); familial myelodysplastic syndrome (MDS); leukemia; sickle-cell anemia; beta-thalassemia; monocytopenia and mycobacterial infections; dendritic cell, nonocyte, B, and natural killer lymphoid deficiency; Emberger syndrome; asymptomatic neurocognitive impairment; mild neurocognitive disorder; and HIV-associated dementia.

### RELATED APPLICATIONS

This application is related to U.S. Provisional Application Ser. No. 62/061,200, filed Oct. 8, 2014, U.S. Provisional Application Ser. No. 62/088,007, filed Dec. 5, 2014, U.S. Provisional Application Ser. No. 62/189,049, filed Jul. 6, 2015, and U.S. Provisional Application Ser. No. 62/195,565, filed Jul. 22, 2015, each of which are incorporated herein by reference in their entirety.

### BACKGROUND

A biological target of recent interest is histone deacetylase (HDAC) (see, for example, a discussion of the use of inhibitors of histone deacetylases for the treatment of cancer: Marks et al. Nature Reviews Cancer 2001, 7, 194; Johnstone et al. Nature Reviews Drug Discovery 2002, 287). Post-translational modification of proteins through acetylation and deacetylation of lysine residues plays a critical role in regulating their cellular functions. HDACs are zinc hydrolases that modulate gene expression through deacetylation of the N-acetyl-lysine residues of histone proteins and other transcriptional regulators (Hassig et al. Curr. Opin. Chem. Biol. 1997, 1, 300-308). HDACs participate in cellular pathways that control cell shape and differentiation, and an HDAC inhibitor has been shown effective in treating an otherwise recalcitrant cancer (Warrell et al. J. Natl. Cancer Inst. 1998, 90, 1621-1625).

There remains a need for identifying the mechanism of action by which HDAC inhibitors act.

### SUMMARY

Provided herein are compounds, pharmaceutical compositions comprising such compounds, and methods of using such compounds to treat diseases or disorders associated with GATA binding protein 2 (Gata2) deficiency, particularly diseases or disorders that involve any type of HDAC1 and/or HDAC2 expression. Such diseases include acute myeloid leukemia (AML); familial myelodysplastic syndrome (MDS); leukemia; sickle-cell anemia; beta-thalassemia; monocytopenia and mycobacterial infections; dendritic cell, nonocyte, B, and natural killer lymphoid deficiency; Emberger syndrome; asymptomatic neurocognitive impairment; mild neurocognitive disorder; and HIV-associated dementia.

In one aspect, provided herein are methods for treating a disease or disorder associated with Gata2 deficiency comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof.

In another aspect, provided herein are methods for increasing Gata2 expression in a cell comprising contacting the cell with a compound of Formula I, II, III, IV, V, VI, VII, VIII, IX, X, or any of the compounds presented in Table 1, Table 2, Table 3, or Table 4, or a pharmaceutically acceptable salt thereof. In some aspects, Gata2 overexpression induces HbG (gamma globin).

In an additional aspect, provided herein are methods for increasing acetylation at Gata2 regulatory regions within a cell comprising contacting the cell with a compound of Formula I, II, III, IV, V, VI, VII, VIII, IX, X, or any of the compounds presented in Table 1, Table 2, Table 3, or Table 4, or a pharmaceutically acceptable salt thereof.

In a further aspect, provided herein are methods for increasing binding of Gata2 to Gata2 regulatory regions within a cell comprising contacting the cell with a compound of Formula I, II, III, IV, V, VI, VII, VIII, IX, X, or any of the compounds presented in Table 1, Table 2, Table 3, or Table 4, or a pharmaceutically acceptable salt thereof.

In one aspect, provided herein is a compound of Formula I:

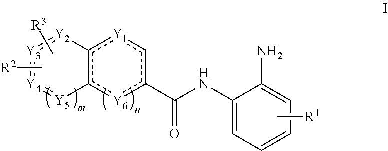

or a pharmaceutically acceptable salt thereof.

In another aspect, provided herein is a compound of Formula II:

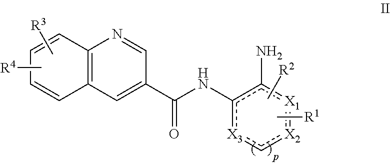

or a pharmaceutically acceptable salt thereof.

In one aspect, provided herein is a compound of Formula IV:

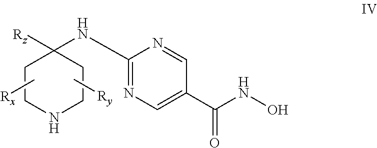

or a pharmaceutically acceptable salt thereof.

In a particular aspect, provided herein is a compound of Formula V:

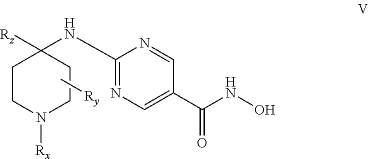

or a pharmaceutically acceptable salt thereof.

In another aspect, provided herein is a compound of Formula VII:

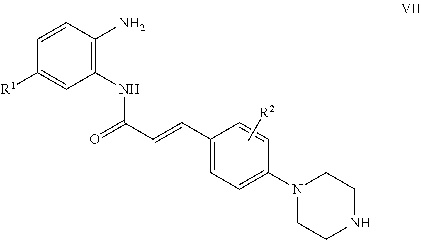

or a pharmaceutically acceptable salt thereof.

In another aspect, provided herein is a compound of Formula VIII:

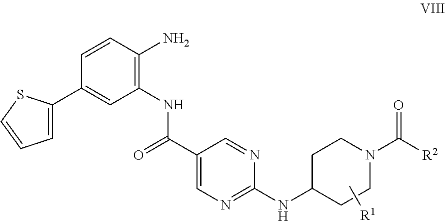

or a pharmaceutically acceptable salt thereof.

In another aspect, provided herein is a compound of Formula IX:

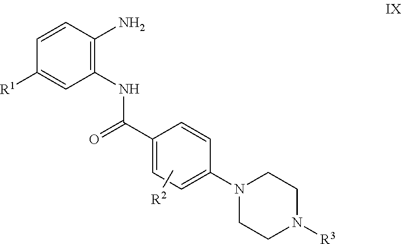

or a pharmaceutically acceptable salt thereof.

In another aspect, provided herein is a compound of Formula X:

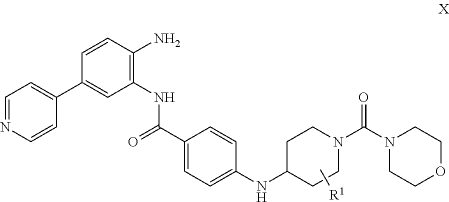

or a pharmaceutically acceptable salt thereof.

In another aspect, provided herein is a pharmaceutical composition comprising a compound of Formula I, Formula II, Formula III, Formula IV, Formula V, Formula VI, Formula VII, Formula VIII, Formula IX, Formula X, or any of the compounds presented in Table 1, Table 2, Table 3, or Table 4, or a pharmaceutically acceptable salt thereof, together with a pharmaceutically acceptable carrier.

In another aspect, provided herein is a method for inhibiting the activity of HDAC1 or HDAC2 in a subject by administering a compound of Formula I, Formula II, Formula III, Formula IV, Formula V, Formula VI, Formula VII, Formula VIII, Formula IX, Formula X, or any of the compounds presented in Table 1, Table 2, Table 3, or Table 4, or a pharmaceutically acceptable salt thereof.

In another aspect, provided herein is a method for selectively inhibiting the activity of HDAC1 or HDAC2 over other HDACs in a subject by administering to the subject a compound of Formula I, Formula II, Formula III, Formula IV, Formula V, Formula VI, Formula VII, Formula VIII, Formula IX, Formula X, or any of the compounds presented in Table 1, Table 2, Table 3, or Table 4, or a pharmaceutically acceptable salt thereof. In some aspects, the compound has a selectivity for HDAC1 over HDAC2. In other aspects, the compound has a selectivity for HDAC2 over HDAC1. In some aspects, the compound has a balanced HDAC1 and HDAC2 selectivity. The term “balanced” means that the selectivity for HDAC1 and HDAC2 is approximately equal, i.e., that the selectivities for HDAC1 and HDAC2 are within about ±10% of each other.

In another aspect, provided herein is a method for inducing histone acetylation within a cell by contacting the cell with either a histone deacetylase 1 (HDAC1) inhibitor or a HDAC2 inhibitor. In some aspects, the HDAC1 inhibitor or the HDAC2 inhibitor is a compound of Formula I, II, III, IV, V, VI, VII, VIII, IX, X, or any of the compounds presented in Table 1, Table 2, Table 3, or Table 4, or a pharmaceutically acceptable salt thereof.

In another aspect, provided herein is a method for inducing HbG (gamma globin) within a cell by contacting the cell with a compound of Formula I, II, III, IV, V, VI, VII, VIII, IX, X, or any of the compounds presented in Table 1, Table 2, Table 3, or Table 4, or a pharmaceutically acceptable salt thereof. In some aspects, the cell is a sickle cell.

In another aspect, provided herein is a method for inducing HbF (fetal hemoglobin) within a cell by contacting the cell with a compound of Formula I, II, III, IV, V, VI, VII, VIII, IX, X, or any of the compounds presented in Table 1, Table 2, Table 3, or Table 4, or a pharmaceutically acceptable salt thereof.

In another aspect, provided herein is a method for attenuating HbG (gamma globin) induction by a histone deacetylase 1 (HDAC1) inhibitor or a HDAC2 inhibitor within a cell comprising contacting the cell with a compound that knocks down GATA binding protein 2 (Gata2).

In another aspect, provided herein is a method for co-occupying the GATA binding protein 2 (Gata2) locus within a cell comprising contacting the cell with a histone deacetylase 1 (HDAC1) inhibitor, an HDAC2 inhibitor, or both an HDAC1 and HDAC2 inhibitor. In some aspects, the HDAC1 inhibitor or HDAC2 inhibitor is a compound of Formula I, II, III, IV, V, VI, VII, VIII, IX, X, or any of the compounds presented in Table 1, Table 2, Table 3, or Table 4, or a pharmaceutically acceptable salt thereof.

In another aspect, provided herein is a method for hyperacetylating histones at GATA binding protein 2 (Gata2) regulatory regions within a cell comprising contacting the cell with a compound of Formula I, II, III, IV, V, VI, VII, VIII, IX, X, or any of the compounds presented in Table 1, Table 2, Table 3, or Table 4, or a pharmaceutically acceptable salt thereof.

In another aspect, provided herein is a method for increasing GATA binding protein 2 (Gata2) at the HbD (delta globin) promoter within a cell comprising contacting the cell with a compound of Formula I, II, III, IV, V, VI, VII, VIII, IX, X, or any of the compounds presented in Table 1, Table 2, Table 3, or Table 4, or a pharmaceutically acceptable salt thereof. In some aspects, increased Gata2 binding at the HbD promoter alters HbG expression.

In a further aspect of the methods of treatment described herein, the subject to be treated is a human.

### BRIEF DESCRIPTION OF THE FIGURES

FIG. 1A is a table showing Affymetrix GeneChip data of mRNA expression changes resulting from Compound 001 treatment or HDAC1 or HDAC2 short hairpin RNA knockdown, relative to untreated controls. NS=not significant, Ratio=fold change resulting from Compound 001 treatment or knockdown.

FIG. 1B is a series of graphs showing quantitative real time PCR (QRT-PCR) data of mRNA expression changes over time resulting from Compound 001 treatment (GATA2 is induced) in culture conditions supporting early erythroblasts derived from CD34+ cells isolated from human bone marrow.

FIG. 1C is a series of graphs showing QRT-PCR data of mRNA expression changes over time resulting from Compound 001 treatment (GATA2 is induced) in culture conditions supporting early erythroblasts derived from CD34+ cells isolated from human bone marrow.

FIG. 1D shows the experimental protocol that was used to generate the data shown in FIG. 1E, FIG. 1F, and FIG. 1G.

FIG. 1E shows a scatter plot of CD71 v. GlyA at Day 0.

FIG. 1F show scatter plots of CD71 v. GlyA for vehicle and Compound 001 at Day 5.

FIG. 1G shows a scatter plot of Compound 001 mRNA v. vehicle mRNA.

FIG. 1H shows the experimental protocol that was used to generate the data shown in FIGS. 1B, 1C and FIG. 1I.

FIG. 1I is a graph that shows the mRNA ratio of Compound 001/vehicle for Gata2, Sox6, Bcl11A, Gata1, Myb, and Klf1 at days 2, 3, 4, 5, 6, and 8.

FIG. 1J shows a gene set enrichment analysis (GSEA) demonstrating that genes up-regulated by HDAC2 knockdown (‘Up in HDAC2 KD’ gene set) are significantly overrepresented at the top of a ranked list of fold changes resulting from Compound 001 treatment.

FIG. 1K shows enrichment scores of the ‘Up in HDAC1 KD’, ‘Up in HDAC2 KD’, and ‘Down in HDAC2 KD’ gene sets relative to all gene sets (2777 total) in the Molecular Signatures Database collection of Chemical and Genetic Perturbations.

FIG. 1L shows CD71 and GlyA surface expression of cells used as input for GeneChip experiments in FIG. 1A, FIG. 1J and FIG. 1K (Exp, experimental replicate); the CD71/GlyA profile for experimental replicate 1 can be found in FIG. 1F (‘vehicle, day 5’ and ‘Compound 001, day 5’).

FIG. 1M shows in the top panel a gene set enrichment analysis demonstrating that genes up-regulated by HDAC1 knockdown (‘Up in HDAC1 KD’ gene set) are significantly overrepresented at the top of a ranked list of fold changes resulting from Compound 001 treatment, and in the bottom panel shows a gene set enrichment analysis demonstrating that genes down-regulated by HDAC2 knockdown (‘Down in HDAC2 KD’ gene set) are significantly overrepresented at the bottom of a ranked list of fold changes resulting from Compound 001 treatment.

FIG. 1N shows a validation of candidate gene expression by QPCR in CS1. Expanded CD34+ bone marrow-derived cells were differentiated in CS1 with 1 μM Compound 001 or vehicle control for the indicated number of days (expression of each candidate gene is relative to β-actin).

FIG. 2A shows that treatment of erythroid progenitors with various HDAC1,2 inhibitors (Compounds 001, 2001 and 2002) leads to induction of Gata2 mRNA (the experimental series was performed on different days and with different donor cells than those of FIG. 2B and FIG. 2C).

FIG. 2B shows that treatment of erythroid progenitors with various HDAC1,2 inhibitors (Compounds 001, Y, 2003, 2004 and 2005) leads to induction of Gata2 mRNA (the experimental series was performed on different days and with different donor cells than those of FIG. 2A and FIG. 2C).

FIG. 2C shows that treatment of erythroid progenitors with various HDAC1,2 inhibitors (Compounds 001, 2007, 2008, 2009 and 2010) leads to induction of Gata2 mRNA (the experimental series was performed on different days and with different donor cells than those of FIG. 2A and FIG. 2B).

FIG. 3 is a graph that shows data for K562 erythroleukemia cells that were treated with Compound 001 for 3 days.

FIG. 4 is a graph that shows that Beta-thalassemia patient samples treated with selective HDAC1,2 inhibitors have elevated levels of Gata2 mRNA.

FIG. 5A shows that sickle cell patient samples (patients 1 and 2) treated with selective HDAC1,2 inhibitors have elevated levels of Gata2 mRNA.

FIG. 5B shows that sickle cell patient samples (patient 3) treated with selective HDAC1,2 inhibitors have elevated levels of Gata2 mRNA.

FIG. 5C shows that sickle cell patient samples (patient 4) treated with selective HDAC1,2 inhibitors have elevated levels of Gata2 mRNA.

FIG. 6A shows overexpression of Gata2 induces gamma globin in erythroid progenitors derived from CD34+ human bone marrow cells. Expanded hematopoietic progenitors were infected with lentivirus carrying the full length Gata2 gene (oeG2) or green fluorescent protein control (oeCtrl). Transduced cells were selected by puromycin treatment and then shifted to culture conditions supporting differentiation of cells into early erythroblasts (Day 0). RNA was isolated at indicated time points and the level of Gata2 mRNA was determined by quantitative real time PCR (QRT-PCR).

FIG. 6B shows the HbG and HbB mRNA levels for the cells in FIG. 6A.

FIG. 7A shows that knockdown of Gata2 mRNA attenuates gamma globin induction by Compound 001. K562 cells were infected with lentivirus, carrying short hairpin RNA targeting Gata2 (shG2-1 and shG2-2) or a non-targeting control short hairpin RNA (shCtrl), as described above. Puromycin was removed (Day 0) and then cells were cultured for an additional three days in the presence of 1 micromolar Compound 001, or vehicle control. RNA was isolated at indicated time point and the level of Gata2 mRNA was determined by quantitative real time PCR (QRT-PCR).

FIG. 7B shows protein levels at Day 3 as determined by Western blot using antibodies against Gata2 and beta-actin as a loading control.

FIG. 7C shows HbG mRNA levels in Compound 001 treated cells. Data was first normalized to beta-actin control and then expressed relative to vehicle treated cells.

FIG. 8A shows Gata2 binding at the beta-like globin gene cluster using ChIP-seq in differentiating primary erythroid progenitors treated with 1 micromolar of Compound 001 or vehicle control. Compound 001 treatment resulted in elevated Gata2 binding at a single region within the beta-like globin gene cluster, located at the delta globin promoter.

FIG. 8B shows an expanded view of data presented in FIG. 8A at the delta globin gene locus showing that Compound 001 treatment results in elevated Gata2 binding near the delta globin promoter.

In FIG. 8C, ChIP-seq results in FIG. 8A were validated in a second experimental series using QRT-PCR and two primer sets directed to the delta globin promoter. A primer set at the beta globin promoter was used as a control.

FIG. 8D shows a proposed mechanism by which HDAC1,2 selective inhibitor induces gamma globin.

FIG. 9A is a graph that shows that Compound 001 was much more selective for HDAC1 (IC₅₀ of 7 nM) and HDAC2 (IC₅₀ of 18 nM) than HDAC3 (IC₅₀ of 1300 nM).

FIG. 9B is a western blot showing that Compound 001, which selectively inhibits HDAC1/2, induced histone acetylation at the indicated sites (H3K9/14ac=histone H3 lysine 9 and 14, H3K56ac=histone H3 lysine 56, H3K79ac=histone H3 lysine 79, H2BK5ac=histone H2B lysine 5, and Total H4=histone H4.

FIG. 10A shows the experimental protocol that was used to generate the data shown in FIG. 10B and FIG. 10C.

FIG. 10B shows a time-dependent increase in the percent HbG mRNA. Expanded CD34+ cells culture in CS1 differentiation media for 8 days with vehicle (dimethyl sulfoxide, DMSO), 30 μM hydroxyurea, 1 μM MS-275, or 1 μM Compound 001 (n=2 QPCR replicates for each of n=2 cell culture replicates, SD).

FIG. 10C shows the percent of HbF containing cells and abundance of HbF per cell are increased in Compound 001 treated cells.

FIG. 11A shows the experimental protocol that was used to generate the data shown in FIG. 11B.

FIG. 11B shows that Compound 001 induced HbG in each of four sickle donor cells.

FIG. 12A shows the experimental protocol that was used to generate the data shown in FIG. 12B, FIG. 12C and FIG. 12D and FIGS. 6A and 6B.

FIG. 12B is a graph that shows Gata2 expression for oeCtrl and oeGata2 on days 0, 3, and 5, and a photo of a western blot showing Gata2 and β-actin for oeCtrl and oeGata2.

FIG. 12C is a scatter plot of cell surface CD71 v. GlyA for cells expressing oeCtrl at day 5, and a scatter plot of cell surface CD71 v. GlyA for cells expressing oeGata2 at day 5.

FIG. 12D is a graph that shows HbG mRNA at days 0, 3, and 5 for oeCtrl and oeGata2.

FIG. 12E shows an effect of Gata2 overexpression on each β-like globin transcript expressed relative to β-actin mRNA.

FIG. 12F shows total β-like globin mRNA (sum of HbB, HbD, HbG, HbE) during erythroid differentiation measured using a QPCR standard curve and then normalized to β-actin levels

FIG. 13A shows the experimental protocol that was used to generate the data shown in FIG. 13B and FIG. 13C.

FIG. 13B is a graph that shows Gata2 mRNA for each of shCtrl, shG2-1, and shG2-2 that was treated with vehicle or Compound 001.

FIG. 13C is a graph that shows HbG mRNA for each of shCtrl, shG2-1, and shG2-2 that was treated with vehicle or Compound 001, and a graph that shows HbB mRNA for each of shCtrl, shG2-1, and shG2-2 that was treated with vehicle or Compound 001.

FIG. 13D shows Gata2 protein levels at day 4.

FIG. 14A shows the experimental protocol that was used to generate the data shown in FIG. 14B.

FIG. 14B shows the location of HDAC1 and HDAC2 binding at the Gata2 gene in CD34+ derived cells and K562 cells.

FIG. 15A shows the experimental protocol that was used to generate the data shown in FIG. 15B.

FIG. 15B are graphs that show histone acetylation levels at various regulatory regions for vehicle or Compound 001 at positions H3K9, H2BK5, and H3K27.

FIG. 16A shows the experimental protocol that was used to generate the data shown in FIG. 16B.

FIG. 16B shows the location of Gata2 binding at the Gata2 gene in CD34+ cells treated with vehicle and Compound 001, and K562 cells.

FIG. 17A shows the chemical structure of the HDAC1/2/3-selective inhibitor Entinostat.

FIG. 17B is a graph that shows that Entinostat is selective for HDAC1 (IC₅₀ of 37 nM), HDAC2 (IC₅₀ of 47 nM) and HDAC3 (IC₅₀ of 95 nM).

FIG. 17C shows the chemical structure of the HDAC1/2-selective inhibitor Compound 001.

FIG. 17D shows that Compound 001 and Entinostat have comparable HDAC2 inhibition activity inside of the cell, using a live cell permeant substrate.

FIG. 18A shows a dose dependent increase in percent HbG mRNA in BFU-E colonies derived from human bone marrow mononuclear cells cultured with Compound 001 (n=2 QPCR replicates for each of n=3 cell culture replicates, SD).

FIG. 18B shows an effect of Compound 001 on each β-like globin transcript. Samples from ‘B’ with each β-like globin transcript plotted relative to β-actin.

FIG. 19A shows that human CD34+ bone marrow cells had a lower level of viability upon treatment with Entinostat when compared to treatment with Compound 001.

FIG. 19B shows representative images of BFU-E colonies following treatment with vehicle, Entinostat (1 μM), or Compound 001 (1 μM).

FIG. 19C shows BFU-E colony counts derived from human bone marrow mononuclear cells plated in vehicle, hydoxyurea (HU), Entinostat (1 μM), or Compound 001 (1 μM).

FIG. 19D shows CFU-GM colony counts derived from human bone marrow mononuclear cells plated in vehicle, hydoxyurea (HU), Entinostat (1 μM), or Compound 001 (1 μM).

FIG. 19E shows erythroid maturation profiles over 8 days using CD71/GlyA following treatment with vehicle, Entinostat (1 μM), or Compound 001 (1 μM).

FIG. 20 shows results of an in vitro HDAC inhibition assay indicating that Entinostat and Compound 001 have negligible inhibitory activity on HDACs 4, 5, 6, 7, 8 and 9 at the concentrations tested.

FIG. 21A shows a dose-dependent increase in the percent HbG and HbE mRNA in CD34+ cells isolated from human bone marrow and cultured in C2 differentiation media for 3 days with Decitabine, Entinostat or Compound 001 (n=3 cell culture replicates).

FIG. 21B shows a comparison of the time-dependent increase in the percent HbG mRNA in CD34+ cells isolated from human bone marrow and cultured in differentiation media for 4 or 5 days with vehicle (DMSO), 1 μM decitabine, or 1 μM Compound 001 (n=2 cell culture replicates, SD).

FIG. 21C shows a comparison of the time-dependent increase in the percent HbG mRNA in CD34+ cells isolated from human bone marrow and cultured in differentiation media for 4 or 5 days with vehicle (DMSO), 30 μM hydroxyurea (HU), 1 μM Entinostat, or 1 μM Compound 001 (n=2 cell culture replicates, SD).

FIG. 21D shows the time-dependent regulation of the β-like globin transcripts from FIG. 10B with each β-hike globin transcript plotted relative to β-actin.

FIG. 21E shows the time-dependent regulation of the β-like globin transcripts from FIG. 21C with each β-like globin transcript plotted relative to β-actin.

FIG. 22 shows CFU-MK and CFU-E colony counts derived from human bone marrow mononuclear cells plated in vehicle, Entinostat (1 μM), or Compound 001 (1 μM) (hydroxyurea (HU) at 10 μM was used as a positive control).

FIG. 23A shows the differentiation stage of cells used for ChIP in FIGS. 14B and 16B. Human CD34+ bone marrow cells were expanded in CS1, then shifted to CS1 differentiation media for 8 days with vehicle or 1 μM Compound 001.

FIG. 23B shows the differentiation stage of cells used for ChIP in FIG. 15B. Human CD34+ bone marrow cells were expanded in CS1, then shifted to CS1 differentiation media for 7 days with vehicle or 1 μM Compound 001.

FIG. 24 shows Compound 005 and azacitidine synergistically induce GATA2 expression in MV4-11 AML cells.

FIG. 25A shows a dosing and blood sampling schedule used to assess the pharmacokinetics of Compound 001 in rats.

FIG. 25B shows a dosing and blood sampling schedule used to assess the pharmacokinetics of Compound 001 in cynomolgus monkeys.

FIG. 25C shows Compound 001 levels in peripheral blood during the 24 hours following the first dose of Compound 001 and at a single point 24 hours following the last dose of Compound 001 for the experiments described in FIG. 25A and FIG. 25B.

FIG. 26A shows white blood cell counts in the rats treated with Compound 001 according to the dosing schedule of FIG. 25A.

FIG. 26B shows white blood cell counts in the monkeys treated with Compound 001 according to the dosing schedule of FIG. 25B.

FIG. 27A shows HbE2 mRNA levels in the rats treated with Compound 001 according to dosing schedule of FIG. 25A.

FIG. 27B shows HbE2 mRNA levels from FIG. 27A for each individual animal at day 6.

FIG. 27C shows HbG mRNA levels in the monkey treated with Compound 001 according to the dosing schedule of FIG. 25B.

FIG. 27D shows HbG mRNA levels from FIG. 27C for each individual animal at day 7.

FIG. 28A shows the dosing schedules used for the data presented in FIGS. 26B and 27C.

FIG. 28B shows the effect of various dosing schedules on embryonic globin (HbE2) mRNA induction in peripheral blood of Rats.

FIG. 28C shows the effect of various dosing schedules on white blood cell counts in peripheral blood of Rats.

FIG. 29 is a graphic representation of heterocellular versus pancellular modes of expression that can result from different dosing and scheduling regimens.

### DETAILED DESCRIPTION

Provided herein are compounds, pharmaceutical compositions comprising such compounds, and methods of using such compounds to treat diseases or disorders associated with GATA binding protein 2 (Gata2) deficiency, particularly diseases or disorders that involve any type of HDAC1 and/or HDAC2 expression. Such diseases include AML, MDS, leukemia, sickle-cell anemia, and beta-thalassemia.

GATA2 has been identified as a new predisposing gene for familial MDS/AML (Hahn, C. N. et al. Nat. Genet. 2011, 43, 929-931; Ostergaard, P. et al. Nat. Genet. 2011, 43, 1012-1017; and R Katherine Hyde & P Paul Liu Nat. Genet. 2011, 43, 926-927). Heterozygous GATA2 germline mutations, both inherited and de novo, have been identified in patients with MDS/AML (Hahn, C. N. et al. Nat. Genet. 2011, 43, 929-931). Most of these mutations have been shown or predicted to result in nonfunctional protein or protein with dominant negative activities. Therefore, restoration of GATA2 function could potentially provide therapeutic benefit for patients with MDS/AML.

### DEFINITIONS

Listed below are definitions of various terms used herein. These definitions apply to the terms as they are used throughout this specification and claims, unless otherwise limited in specific instances, either individually or as part of a larger group.

The term “about” generally indicates a possible variation of no more than 10%, 5%, or 1% of a value. For example, “about 25 mg/kg” will generally indicate, in its broadest sense, a value of 22.5-27.5 mg/kg, i.e., 25±2.5 mg/kg.

The number of carbon atoms in a hydrocarbyl substituent or an alkyl substituent can be indicated by the prefix “C_{x-y},” where x is the minimum and y is the maximum number of carbon atoms in the substituent. Likewise, a Cₓ chain means a hydrocarbyl chain or an alkyl chain containing x carbon atoms.

The term “alkyl” refers to saturated, straight- or branched-chain hydrocarbon moieties containing, in certain embodiments, between one and six (C₁₋₆ alkyl), or one and eight carbon atoms (C₁₋₈ alkyl), respectively. Examples of C₁₋₆ alkyl moieties include, but are not limited to, methyl, ethyl, propyl, isopropyl, n-butyl, tert-butyl, neopentyl, n-hexyl moieties; and examples of C₁₋₈ alkyl moieties include, but are not limited to, methyl, ethyl, propyl, isopropyl, n-butyl, tert-butyl, neopentyl, n-hexyl, heptyl, and octyl moieties.

The term “alkenyl” denotes a monovalent group derived from a hydrocarbon moiety containing, in certain embodiments, from two to six (C₂₋₆ alkenyl), or two to eight carbon atoms having at least one carbon-carbon double bond (C₂₋₈ alkenyl). The double bond may or may not be the point of attachment to another group. Alkenyl groups include, but are not limited to, for example, ethenyl, propenyl, butenyl, 1-methyl-2-buten-1-yl, heptenyl, octenyl and the like.

The term “alkynyl” denotes a monovalent group derived from a hydrocarbon moiety containing, in certain embodiments, from two to six (C₂₋₆ alkynyl), or two to eight carbon atoms having at least one carbon-carbon triple bond (C₂₋₈ alkynyl). The alkynyl group may or may not be the point of attachment to another group. Representative alkynyl groups include, but are not limited to, for example, ethynyl, 1-propynyl, 1-butynyl, heptynyl, octynyl and the like.

The term “alkoxy” refers to an —O-alkyl moiety.

The term “aryl” refers to a mono- or poly-cyclic carbocyclic ring system having one or more aromatic rings, fused or non-fused, including, but not limited to, phenyl, naphthyl, tetrahydronaphthyl, indanyl, idenyl and the like. In some embodiments, aryl groups have 6 carbon atoms. In some embodiments, aryl groups have from six to ten carbon atoms (C₆₋₁₀-aryl). In some embodiments, aryl groups have from six to sixteen carbon atoms (C₆₋₁₆-aryl).

The term “cycloalkyl” denotes a monovalent group derived from a monocyclic or polycyclic saturated or partially unsaturated carbocyclic ring compound. Examples of C₃₋₈-cycloalkyl include, but are not limited to, cyclopropyl, cyclobutyl, cyclopentyl, cyclohexyl, cyclopentyl and cyclooctyl; and examples of C₃-C₁₂-cycloalkyl include, but are not limited to, cyclopropyl, cyclobutyl, cyclopentyl, cyclohexyl, bicyclo[2.2.1]heptyl, and bicyclo[2.2.2]octyl. Also contemplated are groups derived from a monocyclic or polycyclic carbocyclic ring compound having at least one carbon-carbon double bond. Examples of such groups include, but are not limited to, cyclopropenyl, cyclobutenyl, cyclopentenyl, cyclohexenyl, cycloheptenyl, cyclooctenyl, and the like. In some embodiments, cycloalkyl groups have from three to six carbon atoms (C₃₋₆ cycloalkyl). In some embodiments, cycloalkyl groups have from three to eight carbon atoms (C₃₋₈ cycloalkyl).

The term “heteroaryl” refers to a mono- or poly-cyclic (e.g., bi-, or tri-cyclic or more) fused or non-fused moiety or ring system having at least one aromatic ring, having from five to sixteen ring atoms of which one ring atom is selected from oxygen, sulfur, and nitrogen; zero, one or two ring atoms are additional heteroatoms independently selected from oxygen, sulfur, and nitrogen; and the remaining ring atoms are carbon. In some embodiments, the heteroaryl group has from about one to six carbon atoms, and in further embodiments from one to fifteen carbon atoms. In some embodiments, the heteroaryl group contains five to ten ring atoms of which one ring atom is selected from oxygen, sulfur, and nitrogen; zero, one, two, or three ring atoms are additional heteroatoms independently selected from oxygen, sulfur, and nitrogen; and the remaining ring atoms are carbon. Heteroaryl includes, but is not limited to, pyridinyl, pyrazinyl, pyrimidinyl, pyrrolyl, pyrazolyl, imidazolyl, thiazolyl, oxazolyl, isooxazolyl, thiazolyl, thiadiazolyl, oxadiazolyl, thiophenyl, furanyl, indolyl, quinolinyl, isoquinolinyl, benzimidazolyl, benzooxazolyl, quinoxalinyl, acridinyl, and the like.

The term “heterocycloalkyl” refers to a non-aromatic 3-, 4-, 5-, 6- or 7-membered ring or a bi- or tri-cyclic group fused of non-fused system, where (i) each ring contains between one and three heteroatoms independently selected from oxygen, sulfur, and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quaternized, and (iv) any of the above rings may be fused to a benzene ring. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In an embodiment, the heterocycloalkyl group is a 4-7, e.g., 4-6, membered ring.

The terms “halo” and “halogen” refer to an atom selected from fluorine, chlorine, bromine and iodine.

The term “HDAC” refers to histone deacetylases, which are enzymes that remove the acetyl groups from the lysine residues in core histones, thus leading to the formation of a condensed and transcriptionally silenced chromatin. There are currently 18 known histone deacetylases, which are classified into four groups. Class I HDACs, which include HDAC1, HDAC2, HDAC3, and HDAC8, are related to the yeast RPD3 gene. Class II HDACs, which include HDAC4, HDAC5, HDAC6, HDAC7, HDAC9, and HDAC10, are related to the yeast Hda1 gene. Class III HDACs, which are also known as the sirtuins are related to the Sir2 gene and include SIRT1-7. Class IV HDACs, which contains only HDAC11, has features of both Class I and II HDACs. The term “HDAC” refers to any one or more of the 18 known histone deacetylases, unless otherwise specified.

The term “HDAC1/2 selective” means that the compound binds to HDAC1 and HDAC2 to a substantially greater extent, such as 5×, 10×, 15×, 20× greater or more, than to any other type of HDAC enzyme, such as HDAC3 or HDAC6. That is, the compound is selective for HDAC1 and HDAC2 over any other type of HDAC enzyme. For example, a compound that binds to HDAC1 and HDAC2 with an IC₅₀ of 10 nM and to HDAC3 with an IC₅₀ of 50 nM is HDAC1/2 selective. On the other hand, a compound that binds to HDAC1 and HDAC2 with an IC₅₀ of 50 nM and to HDAC3 with an IC₅₀ of 60 nM is not HDAC1/2 selective.

The term “inhibitor” is synonymous with the term antagonist.

The terms “isolated”, “purified”, or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. Particularly, in embodiments the compound is at least 85% pure, more preferably at least 90% pure, more preferably at least 95% pure, and most preferably at least 99% pure.

The term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts provided herein include the conventional non-toxic salts of the parent compound formed, for example, from non-toxic inorganic or organic acids. The pharmaceutically acceptable salts provided herein can be synthesized from the parent compound which contains a basic or acidic moiety by conventional chemical methods. Generally, such salts can be prepared by reaction of the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

Combinations of substituents and variables envisioned by the formulae provided herein are only those that result in the formation of stable compounds. The term “stable” refers to compounds that possess stability sufficient to allow manufacture and that maintain the integrity of the compound for a sufficient period of time to be useful for the purposes detailed herein (e.g., therapeutic or prophylactic administration to a subject).

The term “subject” refers to a mammal. A subject therefore refers to, for example, dogs, cats, horses, cows, pigs, guinea pigs, and the like. Preferably the subject is a human. When the subject is a human, the subject may be referred to herein as a patient.

The terms “treat”, “treating” and “treatment” refer to a method of alleviating or abating a disease and/or its attendant symptoms.

### Compounds

In one aspect, provided herein is a compound of Formula I:

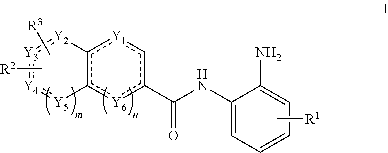

or a pharmaceutically acceptable salt thereof,

wherein

Y₁ is CR⁷ or NR⁷;

Y₂, Y₃, Y₄, Y₅, and Y₆ are each independently CH, CH₂, N, or C(O), wherein at least one of Y₂, Y₃, Y₄, and Y₅ are CH;

R¹ is mono-, bi-, or tri-cyclic aryl or heteroaryl, wherein the mono-, bi-, or tri-cyclic aryl or heteroaryl is optionally substituted one or more times with C₁₋₄-alkyl, CO₂R⁶, C(O)R⁶, or C₁₋₆-alkyl-OR⁶;

R² and R³ are each independently selected from C₂₋₆-alkenyl, C₂₋₆-alkynyl, C₃₋₆-cycloalkyl, C₁₋₆-alkyl-C₃₋₆-cycloalkyl, heterocyclo alkyl, C₁₋₆-alkyl-heterocycloalkyl, NR⁴R⁵, O—C₁₋₆-alkyl-OR⁶, C₁₋₆-alkyl-OR⁶, aryl, heteroaryl, C(O)N(H)-heteroaryl, C(O)-heteroaryl, C(O)-heterocycloalkyl, C(O)-aryl, C(O)—C₁₋₆-alkyl, CO₂—C₁₋₆-alkyl, or C(O)—C₁₋₆-alkyl-heterocycloalkyl, wherein the cycloalkyl, heterocycloalkyl, aryl, or heteroaryl is optionally, independently substituted one or more times with C₁₋₆-alkyl, C₁₋₆-alkoxy, halo, —OH, CO₂R⁶, C(O)R⁶, or C₁₋₆-alkyl-OR⁶.

R⁴ is H, C₁₋₆-alkyl, or C₁₋₆-alkyl-OR⁶;

R⁵ is CO₂R⁶, C₁-C₆-alkyl-aryl, or C₁₋₆-alkyl-OR⁶;

R⁶ is H or C₁₋₆-alkyl;

R⁷ is null, H, C₁₋₆-alkyl, C₃₋₆-cycloalkyl, C₁₋₆-alkyl-C₃₋₆-cyclo alkyl, heterocycloalkyl, or C₁₋₆-alkyl-heterocycloalkyl;

a [symbol] line denotes an optionally double bond;

m is 0 or 1; and

n is 0 or 1, provided at least one of m or n is 1.

In one embodiment of the compound of Formula I, R¹ is mono-, bi-, or tri-cyclic aryl or heteroaryl, wherein the mono-, bi-, or tri-cyclic aryl or heteroaryl is optionally, independently substituted one or more times with halo, C₁₋₄-alkyl, CO₂R⁶, C(O)R⁶, or C₁₋₆-alkyl-OR⁶;

and R² and R³ are each independently selected from C₂₋₆-alkenyl, C₂₋₆-alkynyl, C₃₋₆-cycloalkyl, C₁₋₆-alkyl-C₃₋₆-cycloalkyl, heterocycloalkyl, C₁₋₆-alkyl-heterocycloalkyl, NR⁴R⁵, O—C₁₋₆-alkyl-OR⁶, C₁₋₆-alkyl-OR⁶, aryl, heteroaryl, C(O)N(H)-heteroaryl, C(O)-heteroaryl, C(O)-heterocycloalkyl, C(O)-aryl, C(O)—C₁₋₆-alkyl, CO₂—C₁₋₆-alkyl, and C(O)—C₁₋₆-alkyl-heterocycloalkyl, wherein the cycloalkyl, heterocycloalkyl, aryl, or heteroaryl is optionally, independently substituted one or more times with C₁₋₄-alkyl, CO₂R⁶, C(O)R⁶, or C₁₋₆-alkyl-OR⁶.

In another embodiment of the compound of Formula I, R¹ is monocyclic aryl or heteroaryl, wherein the aryl or heteroaryl is optionally substituted with halo;

R² and R³ are each independently selected from C₂₋₆-alkenyl, C₃₋₆-cycloalkyl, C₁₋₆-alkyl-C₃₋₆-cycloalkyl, heterocycloalkyl, C₁₋₆-alkyl-heterocycloalkyl, NR⁴R⁵, O—C₁₋₆-alkyl-OR⁶, or C₁₋₆-alkyl-OR⁶;

R⁴ is H or C₁₋₆-alkyl;

R⁵ is CO₂R⁶ or C₁₋₆-alkyl-OR⁶; and

R⁶ is C₁₋₆-alkyl.

In one embodiment of the compound of Formula I, m is 1; n is 1; Y₁ is N; and Y₂, Y₃, Y₄, Y₅, and Y₆ are each CH.

In another embodiment of the compound of Formula I, m is 0; n is 1; Y₂ is N; Y₁ is CR⁷; and Y₃, Y₄, and Y₆ are each CH.

In another embodiment of the compound of Formula I, m is 0; n is 1; Y₁ is CR⁷; Y₂ is N; Y₃ is C(O); Y₄ is CH₂; and Y₆ is CH.

In another embodiment of the compound of Formula I m is 1; n is 1; Y₁ is CR⁷; Y₂ is N, and Y₃, Y₄, Y₅, and Y₆ are each CH.

In another embodiment of the compound of Formula I, m is 0; n is 1; Y₁ is CR⁷; Y₂ and Y₃ are each N; and Y₄ and Y₆ are each CH.

In another embodiment of the compound of Formula I, m is 0; n is 1; Y₁ and Y₂ are N; Y₃, Y₄, and Y₆ are each CH.

In yet another embodiment of the compound of Formula I, m is 1; n is 1; and Y₁, Y₂, Y₃, Y₄, Y₅, and Y₆ are each CH.

In one embodiment of the compound of Formula I, R¹ is mono-, bi-, or tri-cyclic aryl or heteroaryl, wherein the mono-, bi-, or tri-cyclic aryl or heteroaryl is optionally, independently substituted one or more times with halo, C₁₋₄-alkyl, CO₂R⁶, C(O)R⁶, or C₁₋₆-alkyl-OR⁶;

and R² and R³ are each independently selected from C₂₋₆-alkenyl, C₂₋₆-alkynyl, C₃₋₆-cycloalkyl, C₁₋₆-alkyl-C₃₋₆-cycloalkyl, heterocycloalkyl, C₁₋₆-alkyl-heterocycloalkyl, NR⁴R⁵, O—C₁₋₆-alkyl-OR⁶, C₁₋₆-alkyl-OR⁶, aryl, heteroaryl, C(O)N(H)-heteroaryl, C(O)-heteroaryl, C(O)-heterocycloalkyl, C(O)-aryl, C(O)—C₁₋₆-alkyl, CO₂—C₁₋₆-alkyl, and C(O)—C₁₋₆-alkyl-heterocycloalkyl, wherein the cycloalkyl, heterocycloalkyl, aryl, or heteroaryl is optionally, independently substituted one or more times with C₁₋₄-alkyl, CO₂R⁶, C(O)R⁶, or C₁₋₆-alkyl-OR⁶.

In another embodiment of the compound of Formula I, R¹ is monocyclic aryl or heteroaryl, wherein the aryl or heteroaryl is optionally substituted with halo;

R² and R³ are each independently selected from C₂₋₆-alkenyl, C₃₋₆-cycloalkyl, C₁₋₆-alkyl-C₃₋₆-cycloalkyl, heterocycloalkyl, C₁₋₆-alkyl-heterocycloalkyl, NR⁴R⁵, O—C₁₋₆-alkyl-OR⁶, or C₁₋₆-alkyl-OR⁶;

R⁴ is H or C₁₋₆-alkyl;

R⁵ is CO₂R⁶ or C₁₋₆-alkyl-OR⁶;

R⁶ is C₁₋₆-alkyl;
m is 1;

n is 1;

Y₁ is N; and

Y₂, Y₃, Y₄, Y₅, and Y₆ are each CH.

In one embodiment of the compound of Formula I, R¹ is mono-, bi-, or tri-cyclic aryl or heteroaryl, wherein the mono-, bi-, or tri-cyclic aryl or heteroaryl is optionally, independently substituted one or more times with halo, C₁₋₄-alkyl, CO₂R⁶, C(O)R⁶, or C₁₋₆-alkyl-OR⁶;

and R² and R³ are each independently selected from C₂₋₆-alkenyl, C₂₋₆-alkynyl, C₃₋₆-cycloalkyl, C₁₋₆-alkyl-C₃₋₆-cycloalkyl, heterocycloalkyl, C₁₋₆-alkyl-heterocycloalkyl, NR⁴R⁵, O—C₁₋₆-alkyl-OR⁶, C₁₋₆-alkyl-OR⁶, aryl, heteroaryl, C(O)N(H)-heteroaryl, C(O)-heteroaryl, C(O)-heterocycloalkyl, C(O)-aryl, C(O)—C₁₋₆-alkyl, CO₂—C₁₋₆-alkyl, and C(O)—C₁₋₆-alkyl-heterocycloalkyl, wherein the cycloalkyl, heterocycloalkyl, aryl, or heteroaryl is optionally, independently substituted one or more times with C₁₋₄-alkyl, CO₂R⁶, C(O)R⁶, or C₁₋₆-alkyl-OR⁶.

In another embodiment of the compound of Formula I, R¹ is monocyclic aryl or heteroaryl, wherein the aryl or heteroaryl is optionally substituted with halo;

R² and R³ are each independently selected from C₂₋₆-alkenyl, C₃₋₆-cycloalkyl, C₁₋₆-alkyl-C₃₋₆-cycloalkyl, heterocycloalkyl, C₁₋₆-alkyl-heterocycloalkyl, NR⁴R⁵, O—C₁₋₆-alkyl-OR⁶, or C₁₋₆-alkyl-OR⁶;

R⁴ is H or C₁₋₆-alkyl;

R⁵ is CO₂R⁶ or C₁₋₆-alkyl-OR⁶;

R⁶ is C₁₋₆-alkyl;

m is 0;

n is 1;

Y₂ is N;

Y₁ is CR⁷; and

Y₃, Y₄, and Y₆ are each CH.

In another embodiment of the compound of Formula I, R¹ is a monocyclic aryl or heteroaryl.

In yet a further embodiment of the compound of Formula I, R¹ is phenyl. R¹ can also be phenyl, wherein phenyl is optionally substituted with halo.

In another embodiment, R¹ is thienyl.

In a further embodiment, R¹ is pyridinyl.

In another embodiment of the compound of Formula I, R¹ is para to NH₂ in the compound of Formula I.

In one embodiment of the compound of Formula I, R² is C₃₋₆-cycloalkyl.

In another embodiment of the compound of Formula I, R² is cyclopropyl. In another embodiment, R² is cyclopentyl.

In a further embodiment of the compound of Formula I R² is C₁₋₆-alkyl-C₃₋₆-cycloalkyl. R² can be CH₂-cyclopropyl.

In a further embodiment of the compound of Formula I, R² is C₂₋₆-alkenyl. For example, R² can be CH₂CH═CH₂.

In an embodiment of the compound of Formula I, R³ is heterocycloalkyl.

In a further embodiment of the compound of Formula I, R³ is morpholinyl or piperazinyl.

In another embodiment of the compound of Formula I, R³ is C₁₋₆-alkyl-heterocycloalkyl. For example, R³ can be CH₂CH₂-morpholinyl, CH₂-morpholinyl, CH₂CH₂-piperazinyl, or CH₂-piperazinyl.

In another embodiment of the compound of Formula I, R³ is O—C₁₋₆-alkyl-OR⁶. For example, R³ can be OCH₂CH₂OCH₃ or OCH₂OCH₃.

In another embodiment of the compound of Formula I, R³ is C₁₋₆-alkyl-OR⁶. For example, R³ can be CH₂CH₂OCH₃.

In a further embodiment of the compound of Formula I, R³ is NR⁴R⁵. For example, R³ can be NHCO₂CH₂CH₃.

In an embodiment of the compound of Formula I, R⁷ is H or C₃₋₆-cycloalkyl. For example, R⁷ can be cyclopropyl.

In another embodiment of Formula I, m is 0; n is 1; Y₂ is N; Y₁ is CR⁷; and Y₃, Y₄, and Y₆ are each CH, and Formula I is of the Formula III:

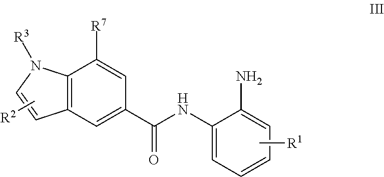

or a pharmaceutically acceptable salt thereof, wherein R¹, R², R³ and R⁷ have the definitions provided above. In an embodiment of Formula III, R² and R³ are each independently selected from C₃₋₆-cyclo alkyl, C₁₋₆-alkyl-C₃₋₆-cycloalkyl, heterocycloalkyl, or C₁₋₆-alkyl-heterocycloalkyl. In another embodiment of Formula III, R⁷ can be H or C₁₋₆-alkyl. In still another embodiment of Formula III, R¹ is R¹ is mono- or bi-cyclic aryl or heteroaryl, wherein the aryl or heteroaryl groups are optionally substituted with halogen. In yet another embodiment of Formula III, R¹ is para to the NH₂ group.

In another embodiment of Formula III, R² and R³ are each independently selected from C₃₋₆-cycloalkyl, C₁₋₆-alkyl-C₃₋₆-cyclo alkyl, heterocycloalkyl, or C₁₋₆-alkyl-heterocycloalkyl; R⁷ can be H or C₁₋₆-alkyl; and R¹ is R¹ is mono- or bi-cyclic aryl or heteroaryl, wherein the aryl or heteroaryl groups are optionally substituted with halogen.

In another aspect, provided herein is a compound of Formula II:

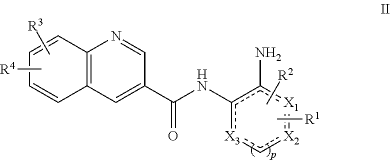

or a pharmaceutically acceptable salt thereof;

wherein

R¹ and R² are independently H, mono-, bi-, or tri-cyclic aryl or heteroaryl, wherein the mono-, bi-, or tri-cyclic aryl or heteroaryl is optionally, independently substituted one or more times with halo, C₁₋₄-alkyl, CO₂R⁷, C(O)R⁷, or C₁₋₆-alkyl-OR⁷;

or R¹ and R² are linked together to form a group of Formula:

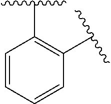 [SMILES: *c1ccccc1*]

R³ and R⁴ are independently selected from H, C₁₋₆-alkyl, C₂₋₆-alkenyl, C₂₋₆-alkynyl, C₃₋₆-cycloalkyl, C₁₋₆-alkyl-C₃₋₆-cycloalkyl, heterocycloalkyl, C₁₋₆-alkyl-heterocycloalkyl, NR⁵R⁶, O—C₁₋₆-alkyl-OR⁷, aryl, heteroaryl, C(O)N(H)-heteroaryl, C(O)-heteroaryl, C(O)-heterocycloalkyl, C(O)-aryl, C(O)—C₁₋₆-alkyl, CO₂—C₁₋₆-alkyl, or C(O)—C₁₋₆-alkyl-heterocycloalkyl, wherein the cycloalkyl, heterocycloalkyl, aryl, or heteroaryl is optionally, independently substituted one or more times with halo, C₁₋₄-alkyl, CO₂R⁷, C(O)R⁷, or C₁₋₆-alkyl-OR⁷;

R⁵ is H, C₁₋₆-alkyl, CO₂R⁷ or C₁₋₆-alkyl-OR⁷;

R⁶ is H, C₁₋₆-alkyl, CO₂R⁷ or C₁₋₆-alkyl-OR⁷;

R⁷ is H or C₁₋₆-alkyl;

X₁, X₂, and X₃ are each independently CH, N, or S, wherein at least one of X₁ or X₂ is N or S;

a [symbol] line denotes an optionally double bond; and

p is 0 or 1.

In one embodiment of the compound of Formula II, R¹ is mono-, bi-, or tri-cyclic aryl or heteroaryl, wherein the mono-, bi-, or tri-cyclic aryl or heteroaryl is optionally, independently substituted one or more times with halo, C₁₋₄-alkyl, CO₂R⁷, C(O)R⁷, or C₁₋₆-alkyl-OR⁷;

R² is H;

or R¹ and R² are linked together to form the following fused ring:

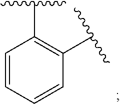 [SMILES: *c1ccccc1*]

and

R³ and R⁴ are independently selected from H, C₁₋₆-alkyl, C₂₋₆-alkenyl, C₂₋₆-alkynyl, C₃₋₆-cycloalkyl, C₁₋₆-alkyl-C₃₋₆-cycloalkyl, heterocycloalkyl, C₁₋₆-alkyl-heterocycloalkyl, NR⁵R⁶, O—C₁₋₆-alkyl-OR⁷, aryl, heteroaryl, C(O)N(H)-heteroaryl, C(O)-heteroaryl, C(O)-heterocycloalkyl, C(O)-aryl, C(O)—C₁₋₆-alkyl, CO₂—C₁₋₆-alkyl, or C(O)—C₁₋₆-alkyl-heterocycloalkyl.

In another embodiment of the compound of Formula II, R¹ is monocyclic aryl or heteroaryl, wherein aryl or heteroaryl is optionally substituted with halo;

R² is H;

or R¹ and R² are linked together to form the following fused ring:

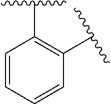 [SMILES: *c1ccccc1*]

R³ is H; and

R⁴ is heterocycloalkyl, wherein the heterocycloalkyl is optionally substituted with C₁₋₄-alkyl, CO₂R⁷, C(O)R⁷, or C₁₋₆-alkyl-OR⁷.

In another embodiment of the compound of Formula II, p is 1; X₁ is N; and X₂ and X₃ are CH.

In another embodiment of the compound of Formula II, p is 1; X₁ and X₂ are CH; and X₃ is N.

In yet another embodiment of the compound of Formula II, p is 1; X₁ and X₃ are CH; and X₂ is N.

In still another embodiment of the compound of Formula II, p is 0; X₁ is S; and X₂ and X₃ are CH.

In still another embodiment of the compound of Formula II p is 0; X₁ and X₂ are CH; and X₃ is S.

In another embodiment of the compound of Formula II, R¹ is monocyclic aryl or heteroaryl, wherein aryl or heteroaryl is optionally substituted with halo;

R² is H;

or R¹ and R² are linked together to form the following fused ring:

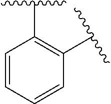 [SMILES: *c1ccccc1*]

R³ is H;

R⁴ is heterocycloalkyl, wherein the heterocycloalkyl is optionally substituted with C₁₋₄-alkyl, CO₂R⁷, C(O)R⁷, or C₁₋₆-alkyl-OR⁷;

p is 1; and

X₁, X₂ and X₃ are CH.

In another embodiment of the compound of Formula II, R¹ is monocyclic aryl or heteroaryl, and the aryl or heteroaryl can be optionally substituted with halo. In another embodiment, R¹ can be phenyl. R¹ can also be thienyl.

In another embodiment of the compound of Formula II, R² is H. In yet another embodiment of the compound of Formula II, R¹ and R² are each H.

In another embodiment of the compound of Formula II, R¹ and R² are linked together to form the following fused ring:

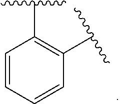 [SMILES: *c1ccccc1*]

In another embodiment of the compound of Formula II, R³ is H.

In another embodiment of the compound of Formula II R⁴ is heterocycloalkyl. R⁴ can be piperazinyl.

In another aspect, provided herein is a compound selected from the group consisting of:

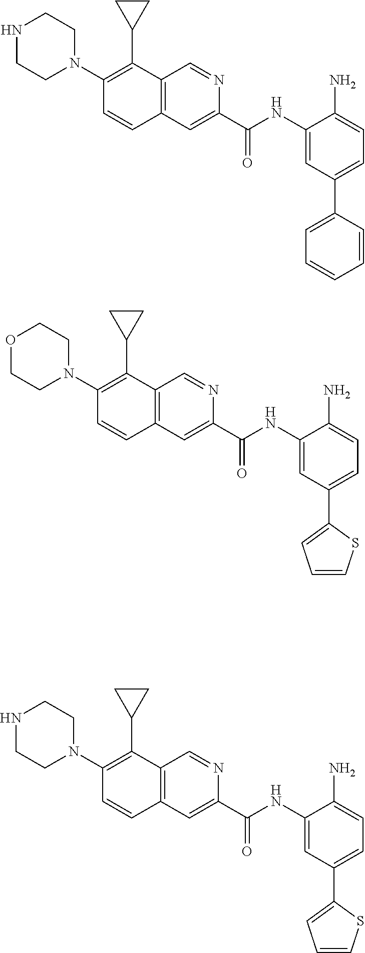 [SMILES: Nc1ccc(-c2ccccc2)cc1NC(=O)c1cc2ccc(N3CCNCC3)c(C3CC3)c2cn1 ; Nc1ccc(-c2cccs2)cc1NC(=O)c1cc2ccc(N3CCOCC3)c(C3CC3)c2cn1 ; Nc1ccc(-c2cccs2)cc1NC(=O)c1cc2ccc(N3CCNCC3)c(C3CC3)c2cn1]

or pharmaceutically acceptable salts thereof.

In one aspect, provided herein is a compound selected from the following compounds of Table 1:

TABLE 1

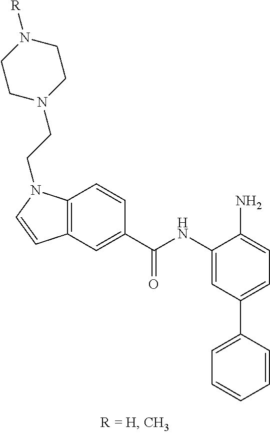

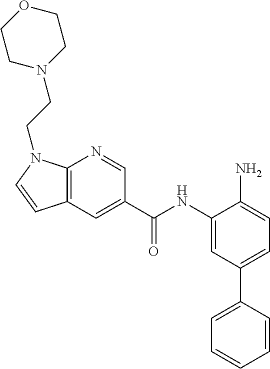 [SMILES: Nc1ccc(-c2ccccc2)cc1NC(=O)c1cnc2c(ccn2CCN2CCOCC2)c1]

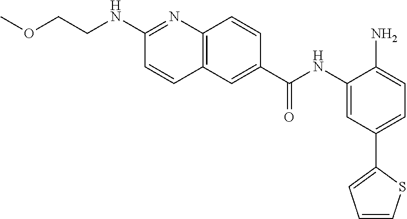 [SMILES: COCCNc1ccc2cc(C(=O)Nc3cc(-c4cccs4)ccc3N)ccc2n1]

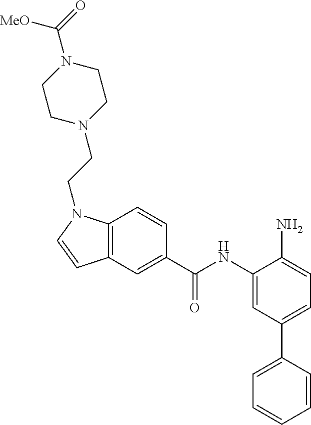 [SMILES: COC(=O)N1CCN(CCn2ccc3cc(C(=O)Nc4cc(-c5ccccc5)ccc4N)ccc32)CC1]

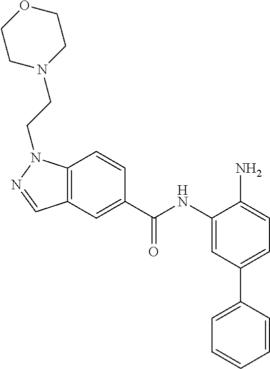 [SMILES: Nc1ccc(-c2ccccc2)cc1NC(=O)c1ccc2c(cnn2CCN2CCOCC2)c1]

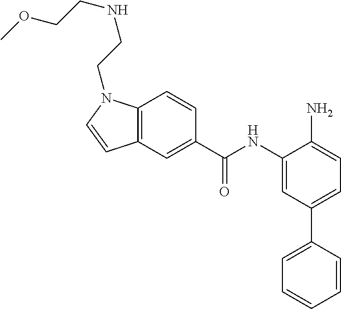 [SMILES: COCCNCCn1ccc2cc(C(=O)Nc3cc(-c4ccccc4)ccc3N)ccc21]

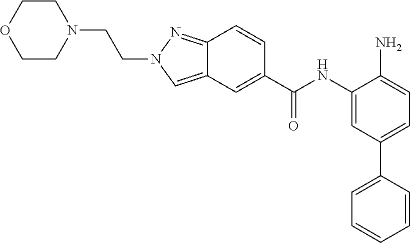 [SMILES: Nc1ccc(-c2ccccc2)cc1NC(=O)c1ccc2nn(CCN3CCOCC3)cc2c1]

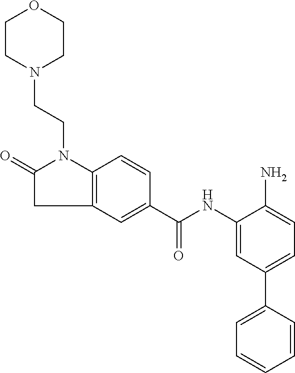 [SMILES: Nc1ccc(-c2ccccc2)cc1NC(=O)c1ccc2c(c1)CC(=O)N2CCN1CCOCC1]

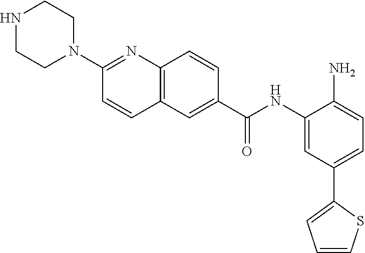 [SMILES: Nc1ccc(-c2cccs2)cc1NC(=O)c1ccc2nc(N3CCNCC3)ccc2c1]

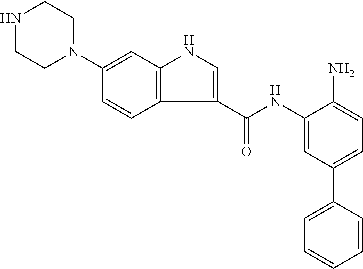 [SMILES: Nc1ccc(-c2ccccc2)cc1NC(=O)c1c[nH]c2cc(N3CCNCC3)ccc12]

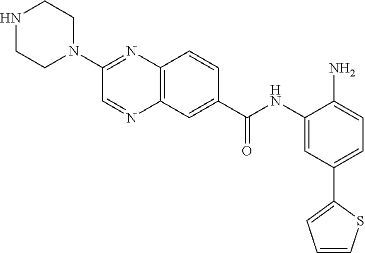 [SMILES: Nc1ccc(-c2cccs2)cc1NC(=O)c1ccc2nc(N3CCNCC3)cnc2c1]

or pharmaceutically acceptable salts thereof.

Representative compounds of the formulae provided herein include, but are not limited to, the following compounds of Table 2.

TABLE 2

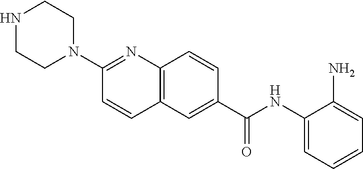 [SMILES: Nc1ccccc1NC(=O)c1ccc2nc(N3CCNCC3)ccc2c1]

N-(2-aminophenyl)-2-(piperazin-1-yl)quinoline-6-
carboxamide
IC₅₀(nM)
HDAC1 = >2,000 HDAC2 = 624
HDAC3 = 104

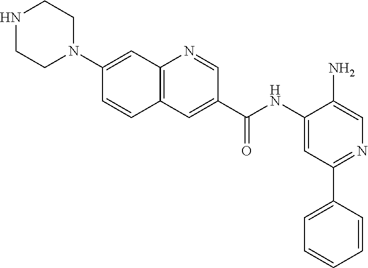 [SMILES: Nc1cnc(-c2ccccc2)cc1NC(=O)c1cnc2cc(N3CCNCC3)ccc2c1]

N-(5-amino-2-phenylpyridin-4-yl)-7-(piperazin-1-
yl)quinoline-3-carboxamide
IC₅₀(nM)
HDAC1 = 1,233 HDAC2 = 1192
HDAC2 = 1876

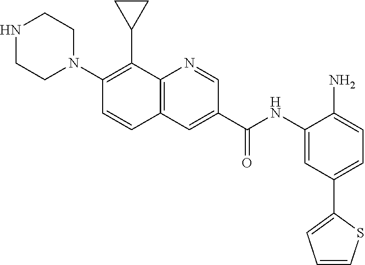 [SMILES: Nc1ccc(-c2cccs2)cc1NC(=O)c1cnc2c(C3CC3)c(N3CCNCC3)ccc2c1]

N-(2-amino-5-(thiophen-2-yl)phenyl)-8-
cyclopropyl-7-(piperazin-1-yl)quinoline-3-
carboxamide
IC₅₀(nM)
HDAC1 = 3.1 HDAC2 = 14
HDAC3 = 99

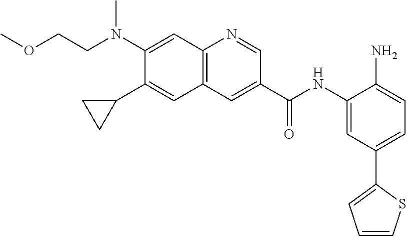 [SMILES: COCCN(C)c1cc2ncc(C(=O)Nc3cc(-c4cccs4)ccc3N)cc2cc1C1CC1]

N-(2-amino-5-(thiophen-2-yl)phenyl)-6-
cyclopropyl-7-((2-
methoxyethyl)(methyl)amino)quinoline-3-
carboxamide
IC₅₀(nM)
HDAC1 = 944 HDAC2 = 667
HDAC3 = >2,000

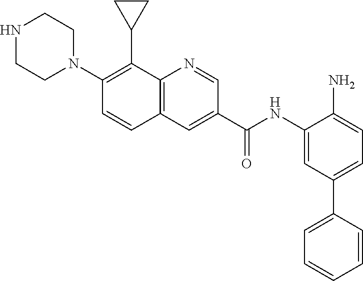 [SMILES: Nc1ccc(-c2ccccc2)cc1NC(=O)c1cnc2c(C3CC3)c(N3CCNCC3)ccc2c1]

N-(4-amino-[1,1′-biphenyl]-3-yl)-8-cyclopropyl-
7-(piperazin-1-yl)quinoline-3-carboxamide
IC₅₀(nM)
HDAC1 = 7.8 HDAC2 = 15
HDAC3 = 164

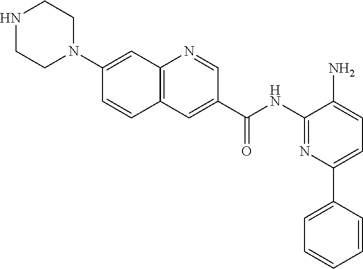 [SMILES: Nc1ccc(-c2ccccc2)nc1NC(=O)c1cnc2cc(N3CCNCC3)ccc2c1]

N-(3-amino-6-phenylpyridin-2-yl)-7-(piperazin-1-
yl)quinoline-3-carboxamide
IC₅₀(nM)
HDAC1 = 1,968 HDAC2 = 336
HDAC3 = 798

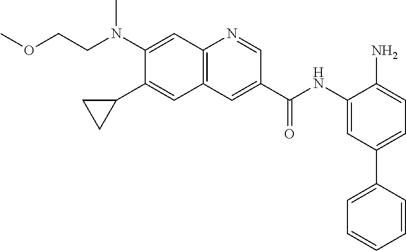 [SMILES: COCCN(C)c1cc2ncc(C(=O)Nc3cc(-c4ccccc4)ccc3N)cc2cc1C1CC1]

N-(4-amino-[1,1′-biphenyl]-3-yl)-6-cyclopropyl-7-
((2-methoxyethyl)(methyl)amino)quinoline-3-
carboxamide
IC₅₀(nM)
HDAC1 = >2,000 HDAC2 = 1220
HDAC3 = >2,000

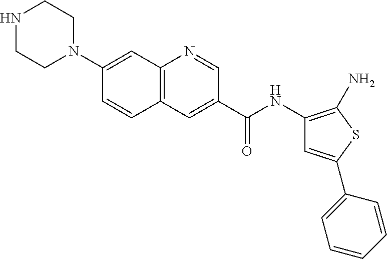 [SMILES: Nc1sc(-c2ccccc2)cc1NC(=O)c1cnc2cc(N3CCNCC3)ccc2c1]

N-(2-amino-5-phenylthiophen-3-yl)-7-(piperazin-1-
yl)quinoline-3-carboxamide
IC₅₀(nM)
HDAC1 = 1210 HDAC2 = 193
HDAC3 = 171

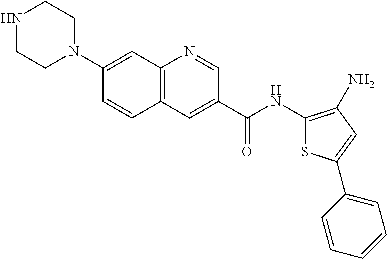 [SMILES: Nc1cc(-c2ccccc2)sc1NC(=O)c1cnc2cc(N3CCNCC3)ccc2c1]

N-(3-amino-5-phenylthiophen-2-yl)-7-(piperazin-
1-yl)quinoline-3-carboxamide
IC₅₀(nM)
HDAC1 = >2,000 HDAC2 = >2,000
HDAC3 = >2,000

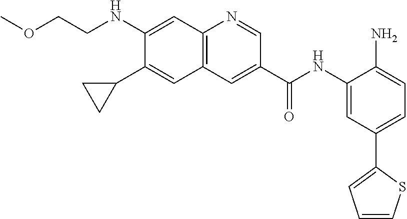 [SMILES: COCCNc1cc2ncc(C(=O)Nc3cc(-c4cccs4)ccc3N)cc2cc1C1CC1]

N-(2-amino-5-(thiophen-2-yl)phenyl)-6-
cyclopropyl-7-((2-methoxyethyl)amino)quinoline-
3-carboxamide
IC₅₀(nM)
HDAC1 = 89 HDAC2 = 243
HDAC3 = 1548

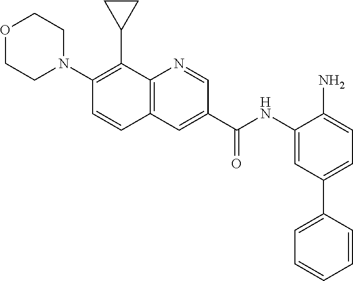 [SMILES: Nc1ccc(-c2ccccc2)cc1NC(=O)c1cnc2c(C3CC3)c(N3CCOCC3)ccc2c1]

N-(4-amino-[1,1′-biphenyl]-3-yl)-8-cyclopropyl-
7-morpholinoquinoline-3-carboxamide
IC₅₀(nM)
HDAC1 = 295 HDAC2 = 799
HDAC3 = >2,000

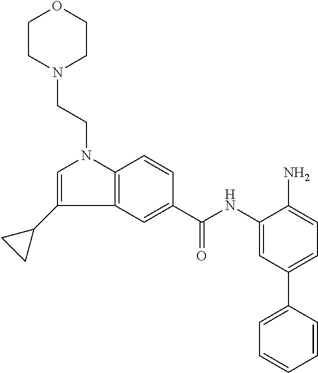 [SMILES: Nc1ccc(-c2ccccc2)cc1NC(=O)c1ccc2c(c1)c(C1CC1)cn2CCN1CCOCC1]

N-(4-amino-[1,1′-biphenyl]-3-yl)-3-cyclopropyl-1-
(2-morpholinoethyl)-1H-indole-5-carboxamide
IC₅₀(nM)
HDAC1 = >2,000 HDAC2 = 681
HDAC3 = 1905

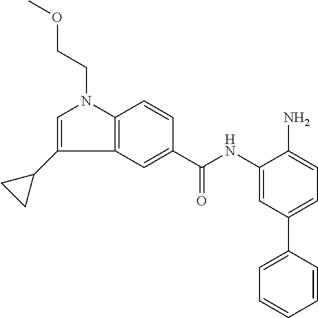 [SMILES: COCCn1cc(C2CC2)c2cc(C(=O)Nc3cc(-c4ccccc4)ccc3N)ccc21]

N-(4-amino-[1,1′-biphenyl]-3-yl)-3-cyclopropyl-1-
(2-methoxyethyl)-1H-indole-5-carboxamide
IC₅₀(nM)
HDAC1 = >2,000 HDAC2 = >2,000
HDAC3 = >2,000

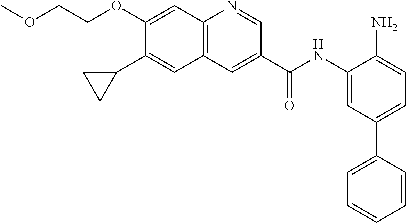 [SMILES: COCCOc1cc2ncc(C(=O)Nc3cc(-c4ccccc4)ccc3N)cc2cc1C1CC1]

N-(4-amino-[1,1′-biphenyl]-3-yl)-6-cyclopropyl-7-
(2-methoxyethoxy)quinoline-3-carboxamide
IC₅₀(nM)
HDAC1 = >2,000 HDAC2 = 1559
HDAC3 = >2,000

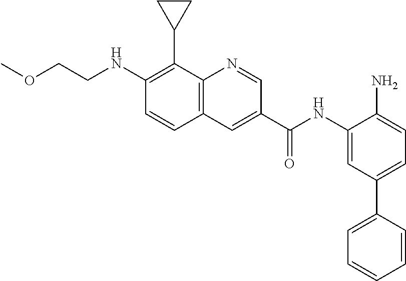 [SMILES: COCCNc1ccc2cc(C(=O)Nc3cc(-c4ccccc4)ccc3N)cnc2c1C1CC1]

N-(4-amino-[1,1′-biphenyl]-3-yl)-8-cyclopropyl-
7-((2-methoxyethyl)amino)quinoline-3-
carboxamide
IC₅₀(nM)
HDAC1 = 115 HDAC2 = 301
HDAC3 = >2,000

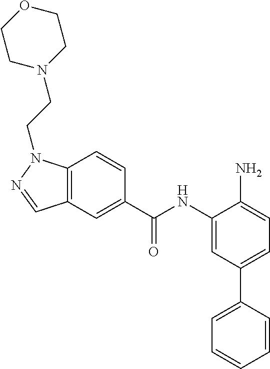 [SMILES: Nc1ccc(-c2ccccc2)cc1NC(=O)c1ccc2c(cnn2CCN2CCOCC2)c1]

N-(4-amino-[1,1′-biphenyl]-3-yl)-1-(2-
morpholinoethyl)-1H-indazole-5-carboxamide
IC₅₀(nM)
HDAC1 = 7.4 HDAC2 = 19
HDAC3 = 344

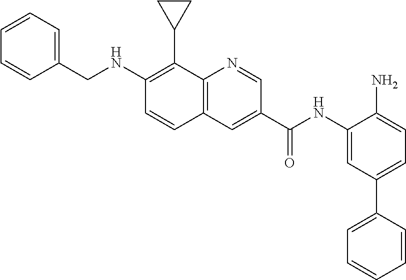 [SMILES: Nc1ccc(-c2ccccc2)cc1NC(=O)c1cnc2c(C3CC3)c(NCc3ccccc3)ccc2c1]

N-(4-amino-[1,1′-biphenyl]-3-yl)-7-(benzylamino)-
8-cyclopropylquinoline-3-carboxamide
IC₅₀(nM)
HDAC1 = 652 HDAC2 = >2,000
HDAC3 = No inhibition

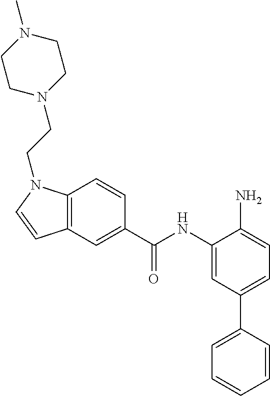 [SMILES: CN1CCN(CCn2ccc3cc(C(=O)Nc4cc(-c5ccccc5)ccc4N)ccc32)CC1]

N-(4-amino-[1,1′-biphenyl]-3-yl)-1-(2-(4-
methylpiperazin-1-yl)ethyl)-1H-indole-5-
carboxamide
IC₅₀(nM)
HDAC1 = 7.1 HDAC2 = 11
HDAC3 = 175

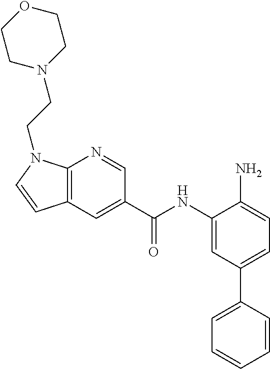 [SMILES: Nc1ccc(-c2ccccc2)cc1NC(=O)c1cnc2c(ccn2CCN2CCOCC2)c1]

N-(4-amino-[1,1′-biphenyl]-3-yl)-1-(2-
morpholinoethyl)-1H-pyrrolo[2,3-b]pyridine-5-
carboxamide
IC₅₀(nM)
HDAC1 = 6.8 HDAC2 = 31
HDAC3 = 373

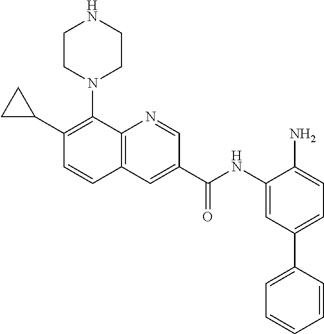 [SMILES: Nc1ccc(-c2ccccc2)cc1NC(=O)c1cnc2c(N3CCNCC3)c(C3CC3)ccc2c1]

N-(4-amino-[1,1′-biphenyl]-3-yl)-7-cyclopropyl-
8-(piperazin-1-yl)quinoline-3-carboxamide
IC₅₀(nM)
HDAC1 = 103 HDAC2 = 56
HDAC3 = 257

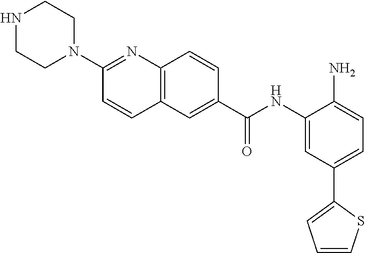 [SMILES: Nc1ccc(-c2cccs2)cc1NC(=O)c1ccc2nc(N3CCNCC3)ccc2c1]

N-(2-amino-5-(thiophen-2-yl)phenyl)-2-
(piperazin-1-yl)quinoline-6-carboxamide,
Compound 001
IC₅₀(nM)
HDAC1 = 4 HDAC2 = 15
HDAC3 = 114

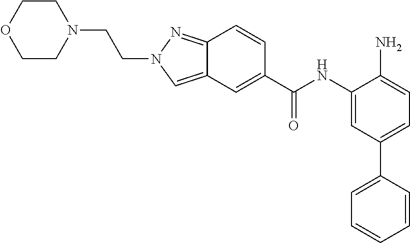 [SMILES: Nc1ccc(-c2ccccc2)cc1NC(=O)c1ccc2nn(CCN3CCOCC3)cc2c1]

N-(4-amino-[1,1′-biphenyl]-3-yl)-2-(2-
morpholinoethyl)-2H-indazole-5-carboxamide
IC₅₀(nM)
HDAC1 = 11 HDAC2 = 23
HDAC3 = 477

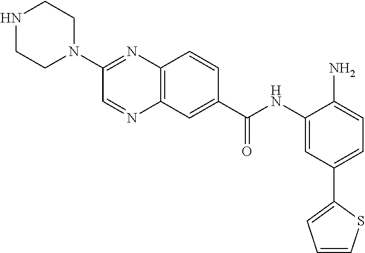 [SMILES: Nc1ccc(-c2cccs2)cc1NC(=O)c1ccc2nc(N3CCNCC3)cnc2c1]

N-(2-amino-5-(thiophen-2-yl)phenyl)-2-(piperazin-
1-yl)quinoxaline-6-carboxamide
IC₅₀(nM)
HDAC1 = 7 HDAC2 = 12
HDAC3 = 71

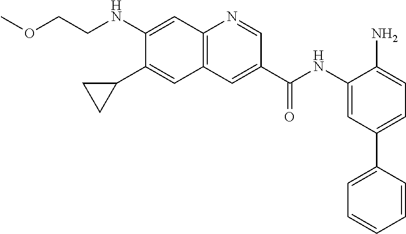 [SMILES: COCCNc1cc2ncc(C(=O)Nc3cc(-c4ccccc4)ccc3N)cc2cc1C1CC1]

N-(4-amino-[1,1′-biphenyl]-3-yl)-6-cyclopropyl-7-
((2-methoxyethyl)amino)quinoline-3-carboxamide

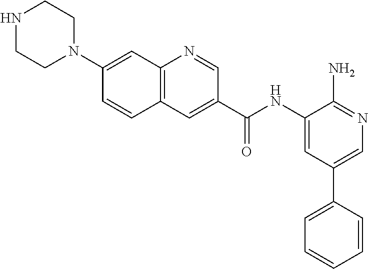 [SMILES: Nc1ncc(-c2ccccc2)cc1NC(=O)c1cnc2cc(N3CCNCC3)ccc2c1]

N-(2-amino-5-phenylpyridin-3-yl)-7-(piperazin-1-
yl)quinoline-3-carboxamide

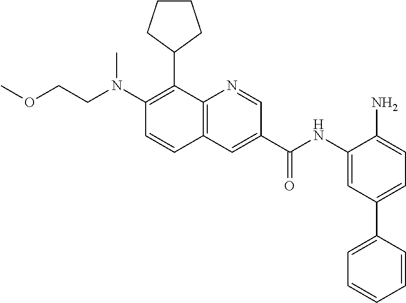 [SMILES: COCCN(C)c1ccc2cc(C(=O)Nc3cc(-c4ccccc4)ccc3N)cnc2c1C1CCCC1]

N-(4-amino-[1,1′-biphenyl]-3-yl)-8-cyclopentyl-7-
((2-methoxyethyl)(methyl)amino)quinoline-3-
carboxamide

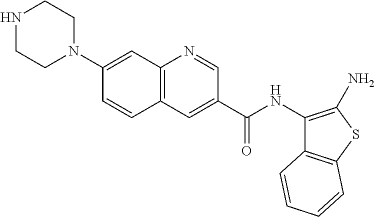 [SMILES: Nc1sc2ccccc2c1NC(=O)c1cnc2cc(N3CCNCC3)ccc2c1]

N-(2-aminobenzo[b]thiophen-3-yl)-7-(piperazin-
1-yl)quinoline-3-carboxamide

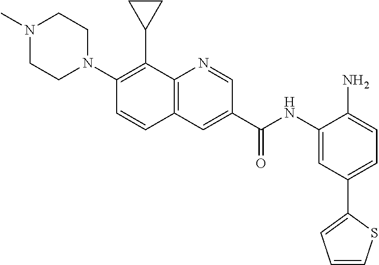 [SMILES: CN1CCN(c2ccc3cc(C(=O)Nc4cc(-c5cccs5)ccc4N)cnc3c2C2CC2)CC1]

N-(2-amino-5-(thiophen-2-yl)phenyl)-8-
cyclopropyl-7-(4-methylpiperazin-1-yl)quinoline-
3-carboxamide

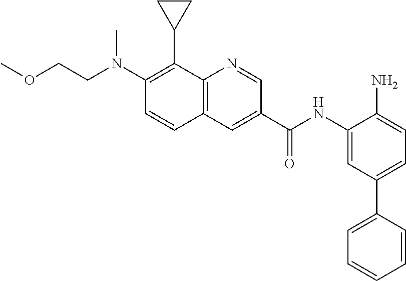 [SMILES: COCCN(C)c1ccc2cc(C(=O)Nc3cc(-c4ccccc4)ccc3N)cnc2c1C1CC1]

N-(4-amino-[1,1′-biphenyl]-3-yl)-8-cyclopropyl-7-
((2-methoxyethyl)(methyl)amino)quinoline-3-
carboxamide

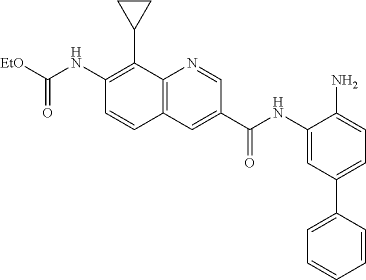 [SMILES: CCOC(=O)Nc1ccc2cc(C(=O)Nc3cc(-c4ccccc4)ccc3N)cnc2c1C1CC1]

ethyl (3-((4-amino-[1,1′-biphenyl]-3-yl)carbamoyl)-
8-cyclopropylquinolin-7-yl)carbamate

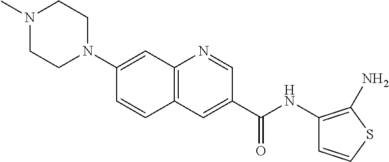 [SMILES: CN1CCN(c2ccc3cc(C(=O)Nc4ccsc4N)cnc3c2)CC1]

N-(2-aminothiophen-3-yl)-7-(4-methylpiperazin-1-
yl)quinoline-3-carboxamide

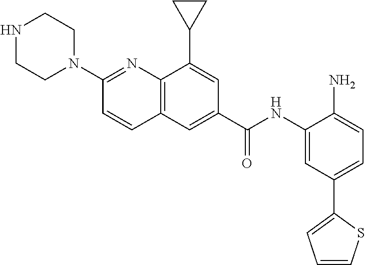 [SMILES: Nc1ccc(-c2cccs2)cc1NC(=O)c1cc(C2CC2)c2nc(N3CCNCC3)ccc2c1]

N-(2-amino-5-(thiophen-2-yl)phenyl)-8-
cyclopropyl-2-(piperazin-1-yl)quinoline-6-
carboxamide

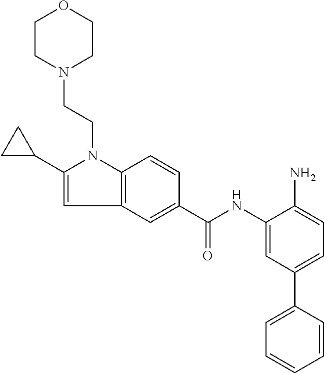 [SMILES: Nc1ccc(-c2ccccc2)cc1NC(=O)c1ccc2c(c1)cc(C1CC1)n2CCN1CCOCC1]

N-(4-amino-[1,1′-biphenyl]-3-yl)-2-cyclopropyl-
1-(2-morpholinoethyl)-1H-indole-5-carboxamide
IC₅₀(nM)
HDAC1 = 15 HDAC2 = 70
HDAC3 = 689

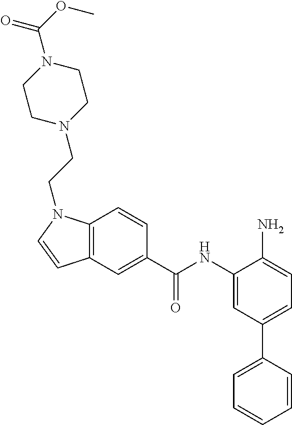 [SMILES: COC(=O)N1CCN(CCn2ccc3cc(C(=O)Nc4cc(-c5ccccc5)ccc4N)ccc32)CC1]

methyl 4-(2-(5-((4-amino-[1,1′-biphenyl]-3-
yl)carbamoyl)-1H-indol-1-yl)ethyl)piperazine-1-
carboxylate

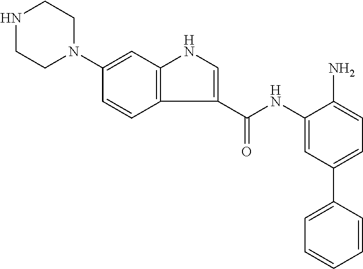 [SMILES: Nc1ccc(-c2ccccc2)cc1NC(=O)c1c[nH]c2cc(N3CCNCC3)ccc12]

N-(4-amino-[1,1′-biphenyl]-3-yl)-6-(piperazin-1-
yl)-1H-indole-3-carboxamide

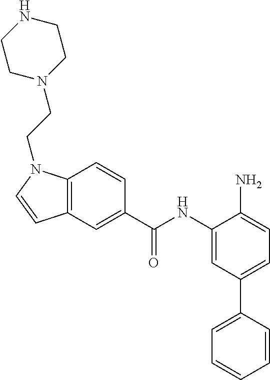 [SMILES: Nc1ccc(-c2ccccc2)cc1NC(=O)c1ccc2c(ccn2CCN2CCNCC2)c1]

N-(4-amino-[1,1′-biphenyl]-3-yl)-1-(2-(piperazin-1-
yl)ethyl)-1H-indole-5-carboxamide

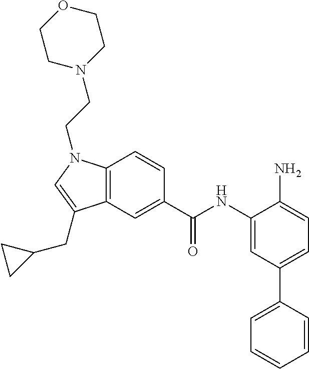 [SMILES: Nc1ccc(-c2ccccc2)cc1NC(=O)c1ccc2c(c1)c(CC1CC1)cn2CCN1CCOCC1]

N-(4-amino-[1,1′-biphenyl]-3-yl)-3-
(cyclopropylmethyl)-1-(2-morpholinoethyl)-1H-
indole-5-carboxamide

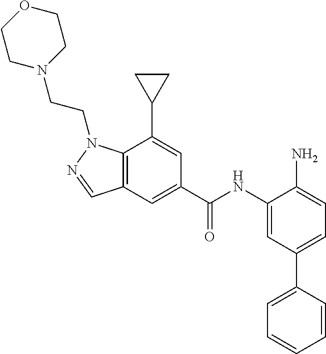 [SMILES: Nc1ccc(-c2ccccc2)cc1NC(=O)c1cc(C2CC2)c2c(cnn2CCN2CCOCC2)c1]

N-(4-amino-[1,1′-biphenyl]-3-yl)-7-cyclopropyl-1-
(2-morpholinoethyl)-1H-indazole-5-carboxamide

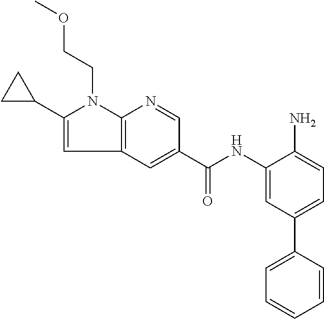 [SMILES: COCCn1c(C2CC2)cc2cc(C(=O)Nc3cc(-c4ccccc4)ccc3N)cnc21]

N-(4-amino-[1,1′-biphenyl]-3-yl)-2-cyclopropyl-
1-(2-methoxyethyl)-1H-indole-5-carboxamide

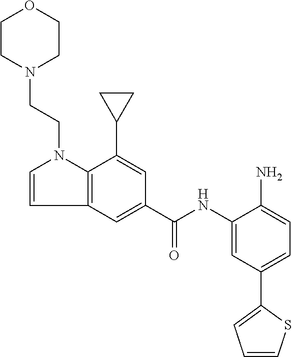 [SMILES: Nc1ccc(-c2cccs2)cc1NC(=O)c1cc(C2CC2)c2c(ccn2CCN2CCOCC2)c1]

N-(2-amino-5-(thiophen-2-yl)phenyl)-7-
cyclopropyl-1-(2-morpholinoethyl)-1H-indole-5-
carboxamide

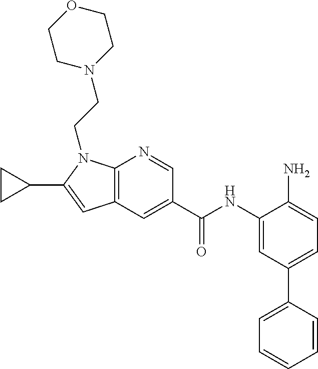 [SMILES: Nc1ccc(-c2ccccc2)cc1NC(=O)c1cnc2c(c1)cc(C1CC1)n2CCN1CCOCC1]

N-(4-amino-[1,1′-biphenyl]-3-yl)-2-cyclopropyl-
1-(2-morpholinoethyl)-1H-pyrrolo[2,3-b]pyridine-
5-carboxamide

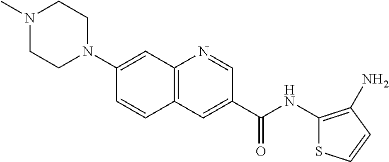 [SMILES: CN1CCN(c2ccc3cc(C(=O)Nc4sccc4N)cnc3c2)CC1]

N-(3-aminothiophen-2-yl)-7-(4-methylpiperazin-
1-yl)quinoline-3-carboxamide

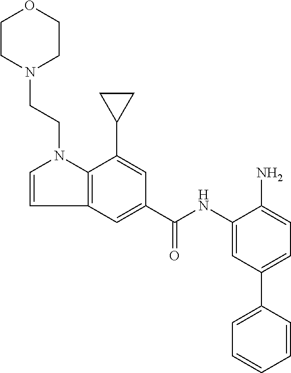 [SMILES: Nc1ccc(-c2ccccc2)cc1NC(=O)c1cc(C2CC2)c2c(ccn2CCN2CCOCC2)c1]

N-(4-amino-[1,1′-biphenyl]-3-yl)-7-cyclopropyl-1-
(2-morpholinoethyl)-1H-indole-5-carboxamide

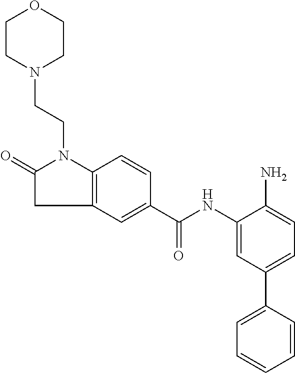 [SMILES: Nc1ccc(-c2ccccc2)cc1NC(=O)c1ccc2c(c1)CC(=O)N2CCN1CCOCC1]

N-(4-amino-[1,1′-biphenyl]-3-yl)-1-(2-
morpholinoethyl)-2-oxoindoline-5-carboxamide

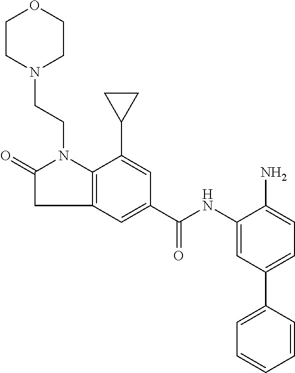 [SMILES: Nc1ccc(-c2ccccc2)cc1NC(=O)c1cc2c(c(C3CC3)c1)N(CCN1CCOCC1)C(=O)C2]

N-(4-amino-[1,1′-biphenyl]-3-yl)-7-cyclopropyl-1-
(2-morpholinoethyl)-2-oxoindoline-5-carboxamide

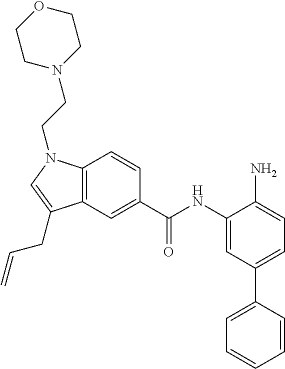 [SMILES: C=CCc1cn(CCN2CCOCC2)c2ccc(C(=O)Nc3cc(-c4ccccc4)ccc3N)cc12]

3-allyl-N-(4-amino-[1,1′-biphenyl]-3-yl)-1-(2-
morpholinoethyl)-1H-indole-5-carboxamide

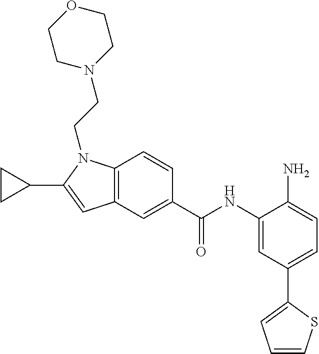 [SMILES: Nc1ccc(-c2cccs2)cc1NC(=O)c1ccc2c(c1)cc(C1CC1)n2CCN1CCOCC1]

N-(2-amino-5-(thiophen-2-yl)phenyl)-2-
cyclopropyl-1-(2-morpholinoethyl)-1H-indole-5-
carboxamide, Compound 005
IC₅₀(nM)
HDAC1 = 6 HDAC2 = 36
HDAC3 = 445

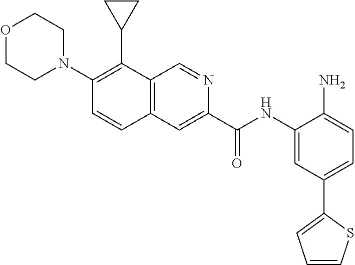 [SMILES: Nc1ccc(-c2cccs2)cc1NC(=O)c1cc2ccc(N3CCOCC3)c(C3CC3)c2cn1]

N-(2-amino-5-(thiophen-2-yl)phenyl)-8-
cyclopropyl-7-morpholinoisoquinoline-3-
carboxamide

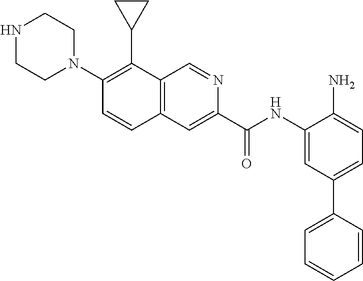 [SMILES: Nc1ccc(-c2ccccc2)cc1NC(=O)c1cc2ccc(N3CCNCC3)c(C3CC3)c2cn1]

N-(4-amino-[1,1′-biphenyl]-3-yl)-8-cyclopropyl-
7-(piperazin-1-yl)isoquinoline-3-carboxamide

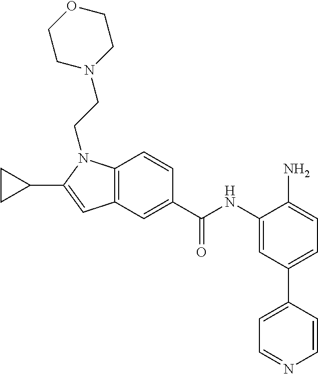 [SMILES: Nc1ccc(-c2ccncc2)cc1NC(=O)c1ccc2c(c1)cc(C1CC1)n2CCN1CCOCC1]

N-(2-amino-5-(pyridin-4-yl)phenyl)-2-cyclopropyl-
1-(2-morpholinoethyl)-1H-indole-5-carboxamide
IC₅₀(nM)
HDAC1 = 27 HDAC2 = 24
HDAC3 = 247

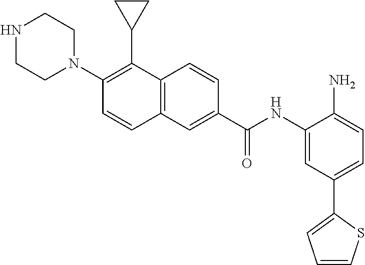 [SMILES: Nc1ccc(-c2cccs2)cc1NC(=O)c1ccc2c(C3CC3)c(N3CCNCC3)ccc2c1]

N-(2-amino-5-(thiophen-2-yl)phenyl)-5-
cyclopropyl-6-(piperazin-1-yl)-2-naphthamide

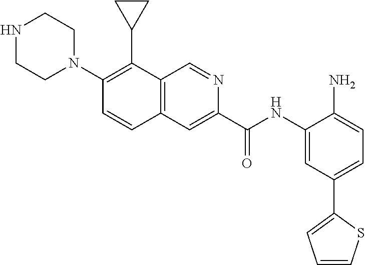 [SMILES: Nc1ccc(-c2cccs2)cc1NC(=O)c1cc2ccc(N3CCNCC3)c(C3CC3)c2cn1]

N-(2-amino-5-(thiophen-2-yl)phenyl)-8-
cyclopropyl-7-(piperazin-1-yl)isoquinoline-3-
carboxamide

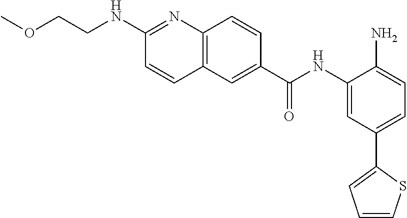 [SMILES: COCCNc1ccc2cc(C(=O)Nc3cc(-c4cccs4)ccc3N)ccc2n1]

N-(2-amino-5-(thiophen-2-yl)phenyl)-2-((2-
methoxyethyl)amino)quinoline-6-carboxamide

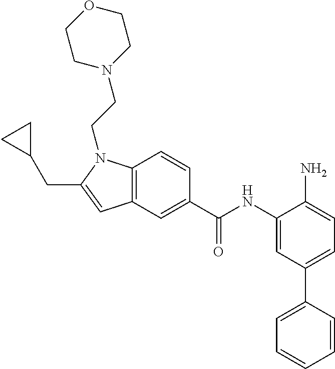 [SMILES: Nc1ccc(-c2ccccc2)cc1NC(=O)c1ccc2c(c1)cc(CC1CC1)n2CCN1CCOCC1]

N-(4-amino-[1,1′-biphenyl]-3-yl)-2-
(cyclopropylmethyl)-1-(2-morpholinoethyl)-1H-
indole-5-carboxamide

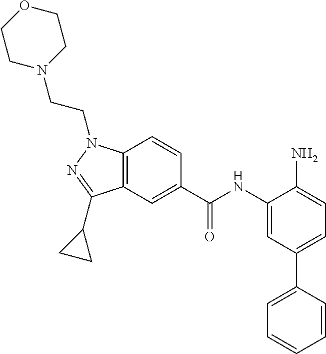 [SMILES: Nc1ccc(-c2ccccc2)cc1NC(=O)c1ccc2c(c1)c(C1CC1)nn2CCN1CCOCC1]

N-(4-amino-[1,1′-biphenyl]-3-yl)-3-cyclopropyl-
1-(2-morpholinoethyl)-1H-indazole-5-
carboxamide

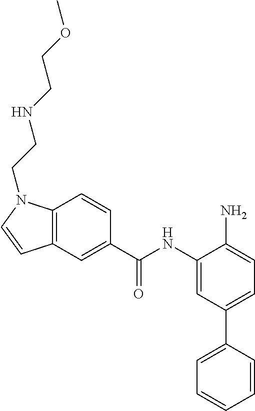 [SMILES: COCCNCCn1ccc2cc(C(=O)Nc3cc(-c4ccccc4)ccc3N)ccc21]

N-(4-amino-[1,1′-biphenyl]-3-yl)-1-(2-((2-
methoxyethyl)amino)ethyl)-1H-indole-5-
carboxamide

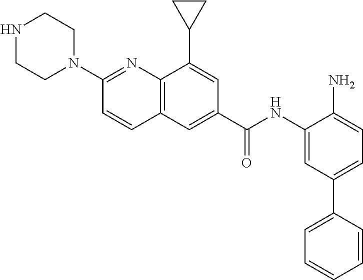 [SMILES: Nc1ccc(-c2ccccc2)cc1NC(=O)c1cc(C2CC2)c2nc(N3CCNCC3)ccc2c1]

N-(4-amino-[1,1′-biphenyl]-3-yl)-8-cyclopropyl-2-
(piperazin-1-yl)quinoline-6-carboxamide

or pharmaceutically acceptable salts thereof.

In one aspect, provided herein is a compound of Formula IV:

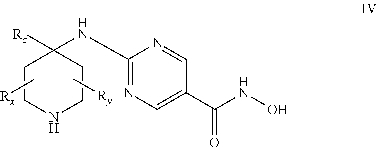

or a pharmaceutically acceptable salt thereof,
wherein,

Rₓ is selected from the group consisting of C₁₋₆-alkyl, C₁₋₆-alkoxy, halo, —OH, —C(O)R¹, —CO₂R¹, —C(O)N(R¹)₂, aryl, —C(S)N(R¹)₂, and S(O)₂R¹, wherein aryl may be optionally substituted by one or more groups selected from C₁₋₆-alkyl, C₁₋₆-alkoxy, —OH, halo, and haloalkyl;

R_{y} is selected from the group consisting of H, C₁₋₆-alkyl, C₁₋₆-alkoxy, halo, —OH, —C(O)R¹, —CO₂R¹, and —C(O)N(R¹)₂;

R_{z} is selected from the group consisting of C₁₋₆-alkyl, C₁₋₆-alkenyl, C₁₋₆-alkynyl, C₃₋₈-cycloalkyl, C₃₋₇-heterocycloalkyl, aryl, and heteroaryl, each of which may be optionally substituted by C₁₋₆-alkyl, C₁₋₆-alkoxy, halo, or —OH; and

each R¹ is, independently for each occurrence, selected from the group consisting of H, C₁₋₆-alkyl, C₃₋₈-cycloalkyl, C₃₋₇-heterocycloalkyl, aryl, heteroaryl, C₁₋₆-alkyl-cyclo alkyl, C₁₋₆-alkyl-heterocycloalkyl, C₁₋₆-alkyl-aryl, and C₁₋₆-alkyl-heteroaryl, wherein C₃₋₈-cyclo alkyl, C₃₋₇-heterocycloalkyl, aryl, heteroaryl, C₁₋₆-alkyl-cyclo alkyl, C₁₋₆-alkyl-heterocycloalkyl, C₁₋₆-alkyl-aryl, and C₁₋₆-alkyl-heteroaryl may be optionally substituted by one or more groups selected from C₁₋₆-alkyl, C₁₋₆-alkoxy, —OH, halo, and haloalkyl.

In an embodiment of the compound of Formula IV or a pharmaceutically acceptable salt thereof,

Rₓ is selected from the group consisting of C₁₋₆-alkyl, C₁₋₆-alkoxy, halo, —OH, —C(O)R¹, —CO₂R¹, and —C(O)N(R¹)₂;

R_{y} is selected from the group consisting of H, C₁₋₆-alkyl, C₁₋₆-alkoxy, halo, —OH, —C(O)R¹, —CO₂R¹, and —C(O)N(R¹)₂;

R_{z} is selected from the group consisting of C₁₋₆-alkyl, C₁₋₆-alkenyl, C₁₋₆-alkynyl, C₃₋₈-cycloalkyl, C₃₋₇-heterocycloalkyl, aryl, and heteroaryl, each of which may be optionally substituted by C₁₋₆-alkyl, C₁₋₆-alkoxy, halo, or —OH; and

each R¹ is, independently for each occurrence, selected from the group consisting of H, C₁₋₆-alkyl, C₃₋₈-cycloalkyl, C₃₋₇-heterocycloalkyl, aryl, heteroaryl, C₁₋₆-alkyl-cyclo alkyl, C₁₋₆-alkyl-heterocycloalkyl, C₁₋₆-alkyl-aryl, and C₁₋₆-alkyl-heteroaryl.

In one embodiment of the compound of Formula IV, provided herein is a compound of Formula V:

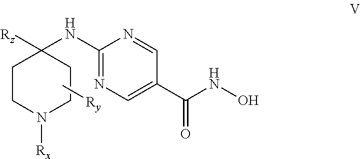

or a pharmaceutically acceptable salt thereof,

wherein,

Rₓ is independently selected from the group consisting of aryl, —C(O)R¹, —CO₂R¹, —C(O)N(R¹)₂, —C(S)N(R¹)₂, and S(O)₂R¹;

R_{y} is selected from the group consisting of H, C₁₋₆-alkyl, or, halo; and

R_{z} is selected from the group consisting of C₁₋₆-alkyl, C₃₋₈-cycloalkyl, C₃₋₇-heterocycloalkyl, aryl, and heteroaryl.

In one embodiment of the compound of Formula V, or a pharmaceutically acceptable salt thereof, Rₓ is independently selected from the group consisting of —C(O)R¹, —CO₂R¹, and —C(O)N(R¹)₂; and R_{z} is selected from the group consisting of C₁₋₆-alkyl, C₃₋₈-cycloalkyl, C₃₋₇-heterocycloalkyl, aryl, and heteroaryl.

In another embodiment of the compounds of Formula IV or V, R_{z} is C₁₋₆-alkyl or aryl. In preferred embodiments of the compounds of Formula IV or V, R_{z} is isopropyl or phenyl. In another embodiment of the compounds of Formula IV or V, R_{z} is methyl.

In a further embodiment of the compounds of Formula IV or V, Rₓ is —C(O)N(R¹)₂ or —C(O)NHR¹. In yet another embodiment of the compounds of Formula IV or V, Rₓ is —C(O)R¹ or —CO₂R¹. In yet another embodiment of the compounds of Formula IV or V, Rₓ is —C(S)N(R¹)₂, —C(S)NHR¹, or S(O)₂R¹.

In an embodiment of the compounds of Formula IV or V, at least one of R¹ is selected from the group consisting of C₁₋₆-alkyl, aryl, C₁₋₆-alkyl-aryl and C₁₋₆-alkyl-heteroaryl, wherein aryl, C₁₋₆-alkyl-aryl and C₁₋₆-alkyl-heteroaryl may be optionally substituted by one or more groups selected from C₁₋₆-alkyl, C₁₋₆-alkoxy, —OH, halo, and haloalkyl. In a further embodiment, R¹ is —CH₃, —CH₂CH₃, phenyl, —CH₂-phenyl, or —CH₂-indolyl, wherein phenyl, —CH₂-phenyl, or —CH₂-indolyl may be optionally substituted by one or more groups selected from C₁₋₆-alkyl or halo.

In another embodiment of the compounds of Formula IV or V, at least one of R¹ is, independently for each occurrence, selected from the group consisting of C₁₋₆-alkyl, aryl, and C₁₋₆-alkyl-aryl. In a further embodiment, at least one of R¹ may be —CH₃, —CH₂CH₃, —CH₂-phenyl, or phenyl.

In another embodiment of the compounds of Formulas IV or V, at least one of R¹ is phenyl, wherein phenyl is optionally substituted by one or more groups selected from C₁₋₆-alkyl, C₁₋₆-alkoxy, halo, and haloalkyl. In preferred embodiments, at least one of R¹ is phenyl, wherein phenyl is optionally substituted by one or more groups selected from CH₃, —OCH₃, fluoro, chloro, and CF₃.

In yet another preferred embodiment of the compounds of Formula IV or V, R_{y} is H.

In another embodiment of the compounds of Formula IV or V, Rₓ is —C(O)R¹; and R¹ is C₁₋₆-alkyl, C₁₋₆-alkyl-aryl or C₁₋₆-alkyl-heteroaryl, wherein C₁₋₆-alkyl-aryl or C₁₋₆-alkyl-heteroaryl may be optionally substituted by one or more groups selected from C₁₋₆-alkyl, C₁₋₆-alkoxy, —OH, halo, and haloalkyl. In a preferred embodiment, R¹ is CH₂-phenyl or CH₂-indolyl, wherein CH₂-phenyl or CH₂-indolyl may be optionally substituted by one or more groups selected from C₁₋₆-alkyl or halo.

In another embodiment of the compound of Formula IV, provided herein is a compound of Formula VI:

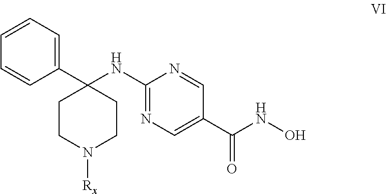

or a pharmaceutically acceptable salt thereof,

wherein,

Rₓ is independently selected from the group consisting of aryl, —C(O)R¹, —CO₂R¹, —C(O)N(R¹)₂, —C(S)N(R¹)₂, and S(O)₂R¹ wherein aryl may be optionally substituted by one or more groups selected from C₁₋₆-alkyl, C₁₋₆-alkoxy, —OH, halo, and haloalkyl; and

each R¹ is, independently for each occurrence, selected from the group consisting of H, C₁₋₆-alkyl, C₃₋₈-cycloalkyl, C₃₋₇-heterocycloalkyl, aryl, heteroaryl, C₁₋₆-alkyl-cyclo alkyl, C₁₋₆-alkyl-heterocycloalkyl, C₁₋₆-alkyl-aryl, and C₁₋₆-alkyl-heteroaryl, wherein C₁₋₆-alkyl, C₃₋₈-cycloalkyl, C₃₋₇-heterocycloalkyl, aryl, heteroaryl, C₁₋₆-alkyl-cycloalkyl, C₁₋₆-alkyl-heterocycloalkyl, C₁₋₆-alkyl-aryl, and C₁₋₆-alkyl-heteroaryl may be optionally substituted by one or more groups selected from C₁₋₆-alkyl, C₁₋₆-alkoxy, halo, and haloalkyl.

In an embodiment of the compounds of Formula VI, Rₓ is —C(O)NHR¹, C(S)NHR¹, or S(O)₂R¹; and

R¹ is, independently for each occurrence, selected from the group consisting of C₁₋₆-alkyl, C₃₋₈-cycloalkyl, C₃₋₇-heterocycloalkyl, aryl, heteroaryl, C₁₋₆-alkyl-cycloalkyl, C₁₋₆-alkyl-heterocycloalkyl, C₁₋₆-alkyl-aryl, and C₁₋₆-alkyl-heteroaryl, wherein C₃₋₈-cycloalkyl, C₃₋₇-heterocycloalkyl, aryl, and heteroaryl may be optionally substituted by one or more groups selected from C₁₋₆-alkyl, C₁₋₆-alkoxy, —OH, halo, and haloalkyl.

In another embodiment of the compounds of Formula VI, at least one of R¹ is selected from the group consisting of C₁₋₆-alkyl, aryl, heteroaryl, C₁₋₆-alkyl-aryl, and C₁₋₆-alkyl-heteroaryl, wherein aryl may be optionally substituted by one or more groups selected from C₁₋₆-alkyl, C₁₋₆-alkoxy, —OH, halo, and haloalkyl.

In another embodiment of the compounds of Formula VI, at least one of R¹ is aryl, wherein aryl is optionally substituted by one or more groups selected from C₁₋₆-alkyl, C₁₋₆-alkoxy, halo, and haloalkyl.

In a preferred embodiment of the compounds of Formula VI, at least one of R¹ is phenyl, wherein phenyl is optionally substituted by one or more groups selected from CH₃, —OCH₃, fluoro, chloro, and CF₃.

In another embodiment of the compounds of Formula VI, Rₓ is —C(O)R¹; and R¹ is C₁₋₆-alkyl, C₁₋₆-alkyl-aryl or C₁₋₆-alkyl-heteroaryl, wherein C₁₋₆-alkyl-aryl or C₁₋₆-alkyl-heteroaryl may be optionally substituted by one or more groups selected from C₁₋₆-alkyl, C₁₋₆-alkoxy, —OH, halo, and haloalkyl. In a preferred embodiment, R¹ is CH₂-phenyl or CH₂-indole, wherein CH₂-phenyl or CH₂-indole may be optionally substituted by one or more groups selected from C₁₋₆-alkyl or halo.

Representative compounds of Formulas IV, V, and VI include, but are not limited to the following compounds of Table 3:

TABLE 3

| | | IC₅₀ (nM) | | | |
|---|---|---|---|---|---|
| ID | Structure | HDAC1 | HDAC2 | HDAC3 | HDAC6 |
| 1001 | 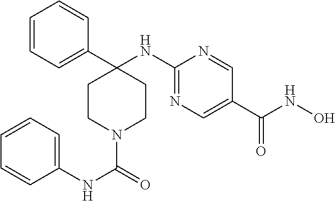 [SMILES: O=C(NO)c1cnc(NC2(c3ccccc3)CCN(C(=O)Nc3ccccc3)CC2)nc1] | 38 | 34 | 1010 | 1.9 |
| 1002 | 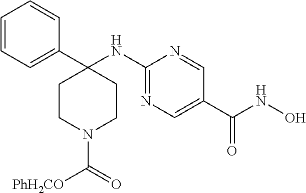 | 1010 | 983 | 1642 | 2.6 |
| 1003 | 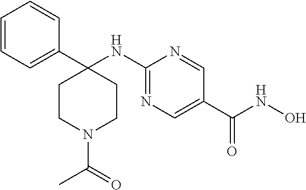 [SMILES: CC(=O)N1CCC(Nc2ncc(C(=O)NO)cn2)(c2ccccc2)CC1] | 346 | 254 | 840 | 1.6 |
| 1004 | 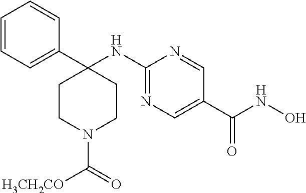 | 275 | 321 | 1003 | 2.9 |
| 1005 | 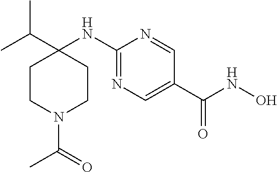 [SMILES: CC(=O)N1CCC(Nc2ncc(C(=O)NO)cn2)(C(C)C)CC1] | 1828 | 2387 | 8180 | 5.9 |
| 1006 | 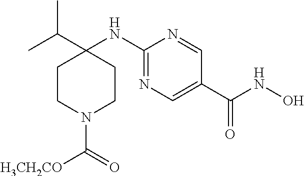 | 697 | 809 | 3781 | 4 |
| 1007 | 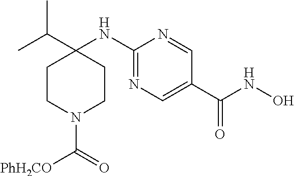 | 119 | 121 | 879 | 5.1 |
| 1008 | 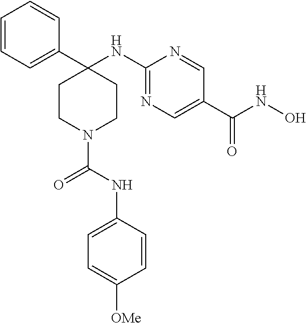 [SMILES: COc1ccc(NC(=O)N2CCC(Nc3ncc(C(=O)NO)cn3)(c3ccccc3)CC2)cc1] | 21 | 24 | 546 | 1.5 |
| 1009 | 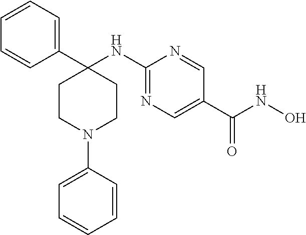 [SMILES: O=C(NO)c1cnc(NC2(c3ccccc3)CCN(c3ccccc3)CC2)nc1] | 356 | 380 | 1785 | 2.1 |
| 1010 | 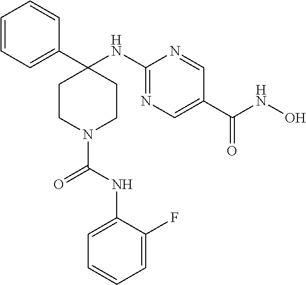 [SMILES: O=C(NO)c1cnc(NC2(c3ccccc3)CCN(C(=O)Nc3ccccc3F)CC2)nc1] | 18 | 27 | 824 | 1.7 |
| 1011 | 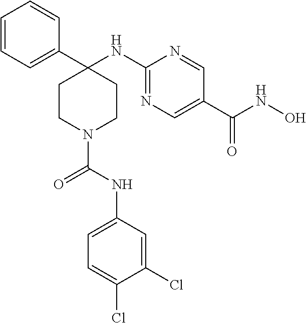 [SMILES: O=C(NO)c1cnc(NC2(c3ccccc3)CCN(C(=O)Nc3ccc(Cl)c(Cl)c3)CC2)nc1] | 110 | 177 | 2164 | 14 |
| 1012 | 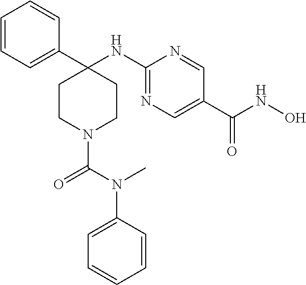 [SMILES: CN(C(=O)N1CCC(Nc2ncc(C(=O)NO)cn2)(c2ccccc2)CC1)c1ccccc1] | 266 | 377 | 1624 | 2.2 |
| 1013 | 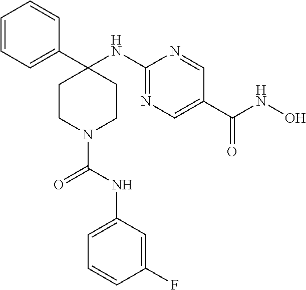 [SMILES: O=C(NO)c1cnc(NC2(c3ccccc3)CCN(C(=O)Nc3cccc(F)c3)CC2)nc1] | 50 | 74 | 1081 | 2.5 |
| 1014 | 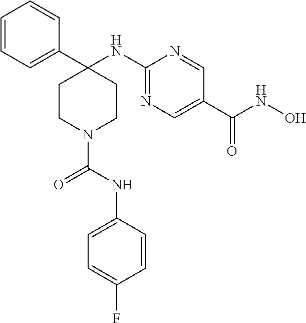 [SMILES: O=C(NO)c1cnc(NC2(c3ccccc3)CCN(C(=O)Nc3ccc(F)cc3)CC2)nc1] | 33 | 43 | 1072 | 2.0 |
| 1015 | 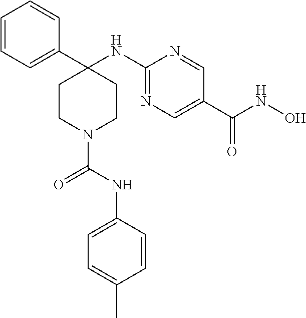 [SMILES: Cc1ccc(NC(=O)N2CCC(Nc3ncc(C(=O)NO)cn3)(c3ccccc3)CC2)cc1] | 34 | 46 | 693 | 2.0 |
| 1016 | 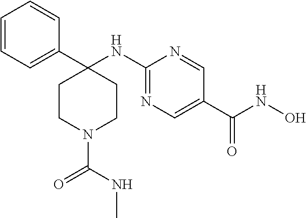 [SMILES: CNC(=O)N1CCC(Nc2ncc(C(=O)NO)cn2)(c2ccccc2)CC1] | 170 | 207 | 987 | 1.7 |
| 1017 | 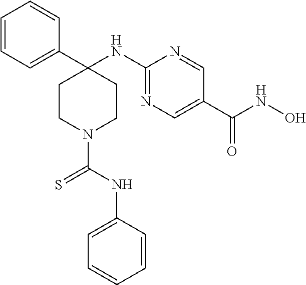 [SMILES: O=C(NO)c1cnc(NC2(c3ccccc3)CCN(C(=S)Nc3ccccc3)CC2)nc1] | 5.9 | 5.2 | 111 | 2.4 |
| 1018 | 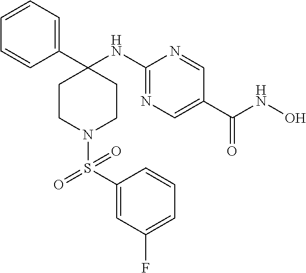 [SMILES: O=C(NO)c1cnc(NC2(c3ccccc3)CCN(S(=O)(=O)c3cccc(F)c3)CC2)nc1] | 551 | 644 | 2485 | 5.1 |
| 1019 | 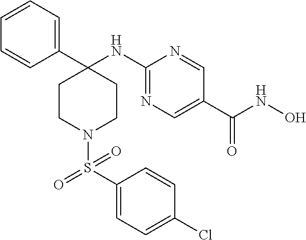 [SMILES: O=C(NO)c1cnc(NC2(c3ccccc3)CCN(S(=O)(=O)c3ccc(Cl)cc3)CC2)nc1] | 854 | 987 | 3190 | 5.0 |
| 1020 | 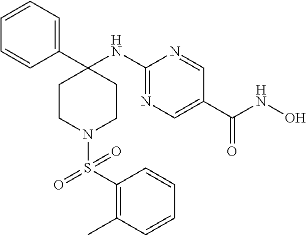 [SMILES: Cc1ccccc1S(=O)(=O)N1CCC(Nc2ncc(C(=O)NO)cn2)(c2ccccc2)CC1] | 372 | 423 | 1983 | 4.5 |
| 1021 | 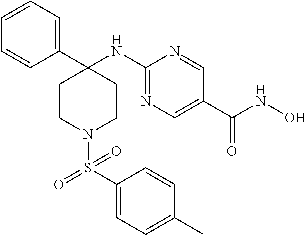 [SMILES: Cc1ccc(S(=O)(=O)N2CCC(Nc3ncc(C(=O)NO)cn3)(c3ccccc3)CC2)cc1] | 570 | 642 | 2513 | 4.5 |
| 1022 | 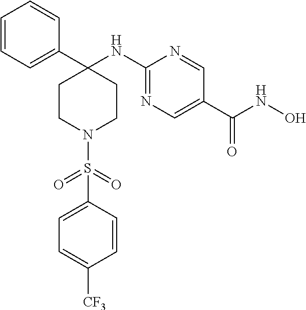 [SMILES: O=C(NO)c1cnc(NC2(c3ccccc3)CCN(S(=O)(=O)c3ccc(C(F)(F)F)cc3)CC2)nc1] | 704 | 782 | 2703 | 7.3 |
| 1023 | 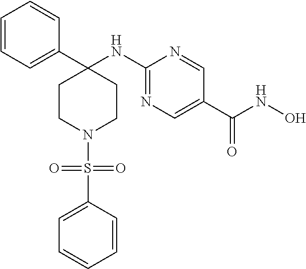 [SMILES: O=C(NO)c1cnc(NC2(c3ccccc3)CCN(S(=O)(=O)c3ccccc3)CC2)nc1] | 844 | 829 | 3545 | 4.6 |
| 1024 | 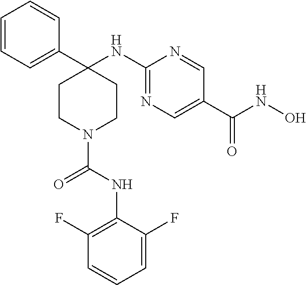 [SMILES: O=C(NO)c1cnc(NC2(c3ccccc3)CCN(C(=O)Nc3c(F)cccc3F)CC2)nc1] | 22 | 22 | 761 | 3.7 |
| 1025 | 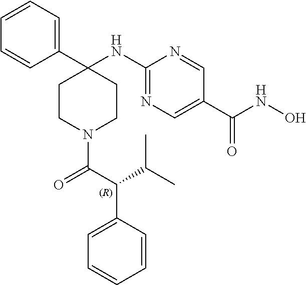 | 20 | 18 | 84 | 13 |
| 1026 | 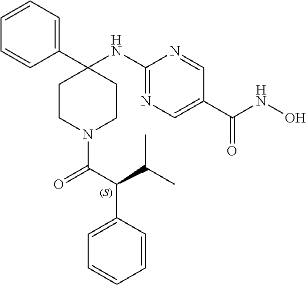 | 206 | 173 | 1100 | 5.0 |
| 1027 | 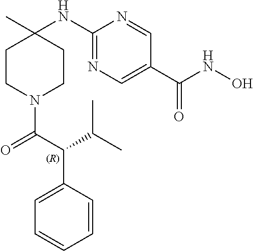 | 130 | 103 | 422 | 12 |
| 1028 | 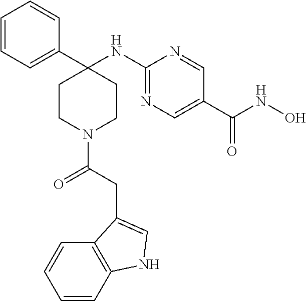 [SMILES: O=C(NO)c1cnc(NC2(c3ccccc3)CCN(C(=O)Cc3c[nH]c4ccccc34)CC2)nc1] | 3 | 2 | 24 | 2.8 |
| 1029 | 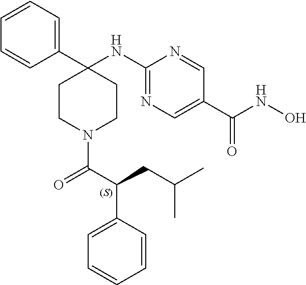 | 102 | 93 | 914 | 11 |
| 1030 | 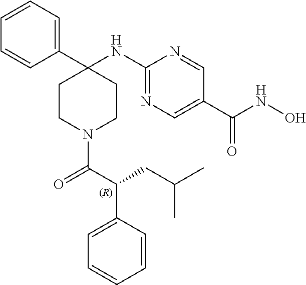 | 23 | 22 | 114 | 12 |
| 1031 | 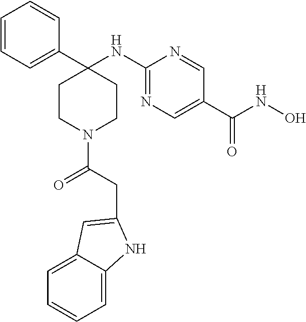 [SMILES: O=C(NO)c1cnc(NC2(c3ccccc3)CCN(C(=O)Cc3cc4ccccc4[nH]3)CC2)nc1] | 10 | 9 | 42 | 5 |

or pharmaceutically acceptable salts thereof.

In another aspect, provided herein is a compound of formula VII:

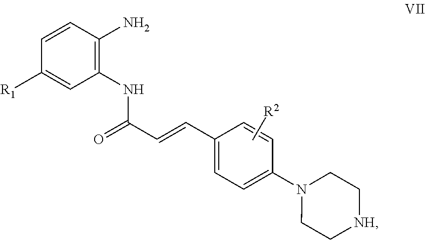

or a pharmaceutically acceptable salt thereof;
wherein

R¹ is phenyl or a 5-membered heteroaryl ring; and

R² is C₃₋₇-cycloalkyl.

In another aspect, provided herein is a compound of formula VIII:

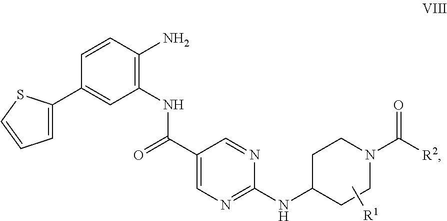

or a pharmaceutically acceptable salt thereof;
wherein

R¹ is C₁₋₄-alkyl; and

R² is a 5- or 6-membered heterocycloalkyl ring optionally substituted with C₁₋₄-alkyl.

In another aspect, provided herein is a compound of formula VIIIa:

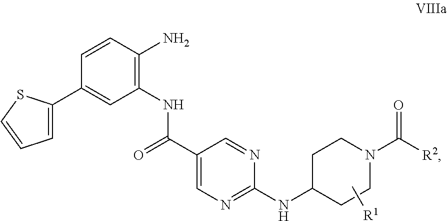

or a pharmaceutically acceptable salt thereof;
wherein

R¹ is H or C₁₋₄-alkyl; and

R² is a 5- or 6-membered heterocycloalkyl ring optionally substituted with C₁₋₄-alkyl.

In another aspect, provided herein is a compound of formula IX:

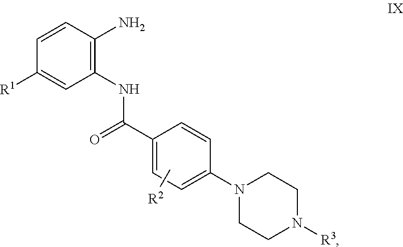

or a pharmaceutically acceptable salt thereof;
wherein

R¹ is selected from H, phenyl, or a 5-membered heteroaryl ring;

R² is C₃₋₇-cycloalkyl; and

R³ is H or C₁₋₄-alkyl.

In another aspect, provided herein is a compound of formula X:

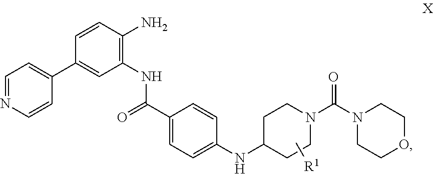

or a pharmaceutically acceptable salt thereof;
wherein

R¹ is C₁₋₄-alkyl.

Representative compounds of Formulas VII, VIII, IX, and X include, but are not limited to the following compounds of Table 4:

TABLE 4

| | | IC₅₀ (nM) | | |
|---|---|---|---|---|
| ID | Structure | HDAC1 | HDAC2 | HDAC3 |
| 2001 | 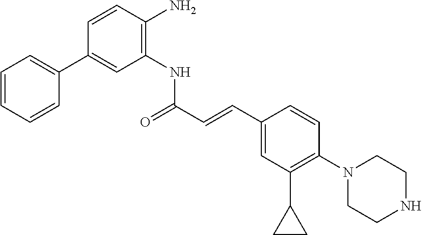 [SMILES: Nc1ccc(-c2ccccc2)cc1NC(=O)/C=C/c1ccc(N2CCNCC2)c(C2CC2)c1] | 27.3 | 32.3 | 218 |
| 2002 | 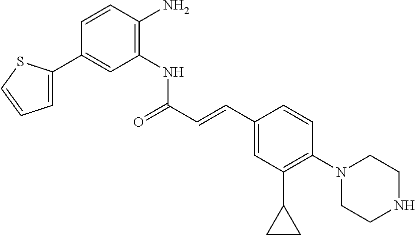 [SMILES: Nc1ccc(-c2cccs2)cc1NC(=O)/C=C/c1ccc(N2CCNCC2)c(C2CC2)c1] | 14.0 | 21.9 | 136 |
| 2003 | 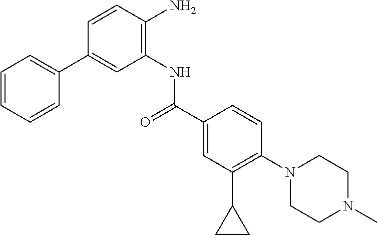 [SMILES: CN1CCN(c2ccc(C(=O)Nc3cc(-c4ccccc4)ccc3N)cc2C2CC2)CC1] | 85.0 | 79.1 | 491 |
| 2004 | 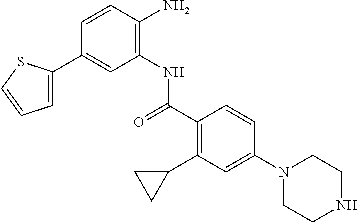 [SMILES: Nc1ccc(-c2cccs2)cc1NC(=O)c1ccc(N2CCNCC2)cc1C1CC1] | 11.2 | 8.9 | 207 |
| 2005 | 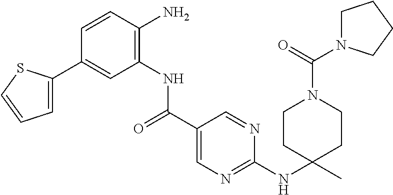 [SMILES: CC1(Nc2ncc(C(=O)Nc3cc(-c4cccs4)ccc3N)cn2)CCN(C(=O)N2CCCC2)CC1] | 10.2 | 14.1 | 673 |
| 2006 | 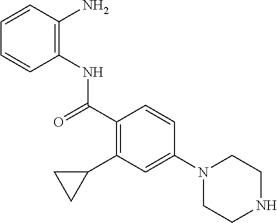 [SMILES: Nc1ccccc1NC(=O)c1ccc(N2CCNCC2)cc1C1CC1] | 1400 | >2000 | 23.0 |
| 2007 | 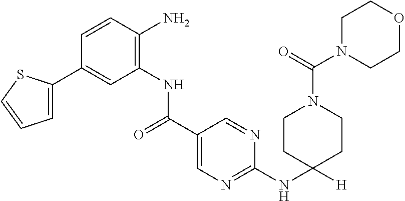 [SMILES: Nc1ccc(-c2cccs2)cc1NC(=O)c1cnc(NC2CCN(C(=O)N3CCOCC3)CC2)nc1] | 14.9 | 55.5 | 284 |
| 2008 | 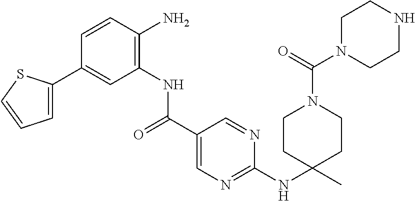 [SMILES: CC1(Nc2ncc(C(=O)Nc3cc(-c4cccs4)ccc3N)cn2)CCN(C(=O)N2CCNCC2)CC1] | 7.27 | 25.9 | 195 |
| 2009 | 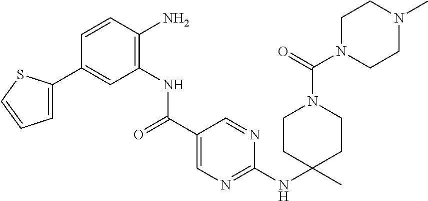 [SMILES: CN1CCN(C(=O)N2CCC(C)(Nc3ncc(C(=O)Nc4cc(-c5cccs5)ccc4N)cn3)CC2)CC1] | 8.53 | 31.5 | 259 |
| 2010 | 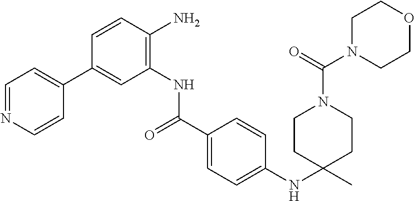 [SMILES: CC1(Nc2ccc(C(=O)Nc3cc(-c4ccncc4)ccc3N)cc2)CCN(C(=O)N2CCOCC2)CC1] | 12.0 | 16.8 | 511 |

or pharmaceutically acceptable salts thereof.

In preferred embodiments, the compounds of provided herein have one or more of the following properties: the compound is capable of inhibiting at least one histone deacetylase (HDAC); the compound is capable of inhibiting HDAC1 and/or HDAC2; the compound is a selective HDAC1 and/or HDAC2 inhibitor.

In another aspect, provided herein is a method of synthesizing a compound of Formula I, II, III, IV, V, VI, VII, VIII, IX, X, or any of the compounds presented in Table 1, Table 2, Table 3, or Table 4. The synthesis of the compounds provided herein can be found in the Examples below.

Another embodiment is a method of making a compound of any of the formulae herein using any one of, or combination of, the reactions delineated herein. The method can include the use of one or more intermediates or chemical reagents delineated herein.

Another aspect is an isotopically labeled compound of any of the formulae delineated herein. Such compounds have one or more isotope atoms that may or may not be radioactive (e.g., ³H, ²H, ¹⁴C, ¹³C, ³⁵S, ³²P, ¹²⁵I, and ¹³¹I) introduced into the compound. Such compounds are useful for drug metabolism studies and diagnostics, as well as therapeutic applications.

Compounds provided herein can be conveniently prepared, or formed during the processes provided herein, as solvates (e.g., hydrates). Hydrates of compounds provided herein can be conveniently prepared by recrystallization from an aqueous/organic solvent mixture, using organic solvents such as dioxan, tetrahydrofuran or methanol.

Some of the compounds provided herein have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms, and other stereoisomeric forms that may be defined, in terms of absolute stereochemistry, as (R)- or (S)-, or as (D)- or (L)- for amino acids. All such isomeric forms of these compounds are expressly included herein. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). The compounds provided herein may also be represented in multiple tautomeric forms. In such instances, all tautomeric forms of the compounds described herein are expressly included. When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration unless the text so states; thus a carbon-carbon double bond depicted arbitrarily herein as trans may be cis, trans, or a mixture of the two in any proportion. All such isomeric forms of such compounds are expressly included herein. All crystal forms of the compounds described herein are expressly included herein.

The compounds provided herein are defined herein by their chemical structures and/or chemical names. Where a compound is referred to by both a chemical structure and a chemical name, and the chemical structure and chemical name conflict, the chemical structure is determinative of the compound's identity.

### Pharmaceutical Compositions

Provided herein are pharmaceutical compositions comprising a compound provided herein, or a pharmaceutically acceptable salt thereof, together with a pharmaceutically acceptable carrier.

In another aspect, provided herein is a pharmaceutical composition comprising any of the compounds provided herein (Formula I, II, III, IV, V, VI, VII, VIII, IX, X, or any of the compounds presented in Table 1, Table 2, Table 3, or Table 4) or a pharmaceutically acceptable salt, thereof, together with a pharmaceutically acceptable carrier.

The pharmaceutical compositions provided herein comprise a therapeutically effective amount of a compound provided herein formulated together with one or more pharmaceutically acceptable carriers. The term “pharmaceutically acceptable carrier” means a non-toxic, inert solid, semi-solid or liquid filler, diluent, encapsulating material or formulation auxiliary of any type. The pharmaceutical compositions provided herein can be administered to humans and other animals orally, rectally, parenterally, intracisternally, intravaginally, intraperitoneally, topically (as by powders, ointments, or drops), buccally, or as an oral or nasal spray.

The compounds provided herein can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound provided herein in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound provided herein with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

### Methods

In one aspect, provided herein is a method for treating a disease or disorder associated with Gata2 deficiency comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof. Such diseases include acute myeloid leukemia (AML); familial myelodysplastic syndrome (MDS); leukemia; sickle-cell anemia; beta-thalassemia; monocytopenia and mycobacterial infections; dendritic cell, nonocyte, B, and natural killer lymphoid deficiency; Emberger syndrome; asymptomatic neurocognitive impairment; mild neurocognitive disorder; and HIV-associated dementia. In an embodiment, the compound is an HDAC1/2 selective inhibitor.

In one aspect, provided herein is a method for treating a disease or disorder associated with Gata2 deficiency comprising administering to a subject in need thereof a therapeutically effective amount of an HDAC1 inhibitor.

In one aspect, provided herein is a method for treating a disease or disorder associated with Gata2 deficiency comprising administering to a subject in need thereof a therapeutically effective amount of an HDAC1/2 selective inhibitor.

In another aspect, provided herein are methods for increasing Gata2 expression in a cell comprising contacting the cell with an HDAC1 inhibitor. In some aspects, Gata2 overexpression induces HbG (gamma globin).

In an additional aspect, provided herein are methods for increasing acetylation at Gata2 regulatory regions within a cell comprising contacting the cell with an HDAC1 inhibitor.

In a further aspect, provided herein are methods for increasing binding of Gata2 to Gata2 regulatory regions within a cell comprising contacting the cell with an HDAC1 inhibitor.

In one aspect, provided herein is a method for treating a disease or disorder associated with Gata2 deficiency comprising administering to a subject in need thereof a therapeutically effective amount of an HDAC2 inhibitor.

In another aspect, provided herein are methods for increasing Gata2 expression in a cell comprising contacting the cell with an HDAC2 inhibitor. In some aspects, Gata2 overexpression induces HbG (gamma globin).

In an additional aspect, provided herein are methods for increasing acetylation at Gata2 regulatory regions within a cell comprising contacting the cell with an HDAC2 inhibitor.

In a further aspect, provided herein are methods for increasing binding of Gata2 to Gata2 regulatory regions within a cell comprising contacting the cell with an HDAC2 inhibitor.

In another aspect, provided herein is a method for increasing Gata2 expression in a cell comprising contacting the cell with a compound of Formula I, II, III, IV, V, VI, VII, VIII, IX, X, or any of the compounds presented in Table 1, Table 2, Table 3, or Table 4, or a pharmaceutically acceptable salt thereof. In some aspects, Gata2 overexpression induces HbG (gamma globin).

In an additional aspect, provided herein is a method for increasing acetylation at Gata2 regulatory regions within a cell comprising contacting the cell with a compound of Formula I, II, III, IV, V, VI, VII, VIII, IX, X, or any of the compounds presented in Table 1, Table 2, Table 3, or Table 4, or a pharmaceutically acceptable salt thereof.

In a further aspect, provided herein is a method for increasing binding of Gata2 to Gata2 regulatory regions within a cell comprising contacting the cell with a compound of Formula I, II, III, IV, V, VI, VII, VIII, IX, X, or any of the compounds presented in Table 1, Table 2, Table 3, or Table 4, or a pharmaceutically acceptable salt thereof.

In one aspect, provided herein is a method of selectively inhibiting HDAC1 or HDAC2 over other HDACs in a subject comprising administering a compound of Formula I, II, II, IV, V, VI, or any of the compounds presented in Table 1, Table 2, Table 3, or Table 4, and pharmaceutically acceptable salts thereof. In some embodiments, the compound has a selectivity for HDAC1 over HDAC2. In other embodiments, the compound has a selectivity for HDAC2 over HDAC1. In some embodiments, the compound has a balanced HDAC1 and HDAC2 selectivity. The term “balanced” means that the selectivity for HDAC1 and HDAC2 is approximately equal, i.e., that the selectivities for HDAC1 and HDAC2 are within about ±10% of each other.

In another aspect, provided herein is a method for inducing histone acetylation within a cell by contacting the cell with either a histone deacetylase 1 (HDAC1) inhibitor or a HDAC2 inhibitor. In some aspects, the HDAC1 inhibitor or the HDAC2 inhibitor is a compound of Formula I, II, III, IV, V, VI, VII, VIII, IX, X, or any of the compounds presented Table 1, Table 2, Table 3, or Table 4, or a pharmaceutically acceptable salt thereof.

In another aspect, provided herein is a method for inducing HbG (gamma globin) within a cell by contacting the cell with a compound of Formula I, II, III, IV, V, VI, VII, VIII, IX, X, or any of the compounds presented in Table 1, Table 2, Table 3, or Table 4, or a pharmaceutically acceptable salt thereof. In some aspects, the cell is a sickle cell.

In another aspect, provided herein is a method for inducing HbF (fetal hemoglobin) within a cell by contacting the cell with a compound of Formula I, II, III, IV, V, VI, VII, VIII, IX, X, or any of the compounds presented in Table 1, Table 2, Table 3, or Table 4, or a pharmaceutically acceptable salt thereof.

In another aspect, provided herein is a method for attenuating HbG (gamma globin) induction by a histone deacetylase 1 (HDAC1) inhibitor or a HDAC2 inhibitor within a cell comprising contacting the cell with a compound that knocks down GATA binding protein 2 (Gata2).

In another aspect, provided herein is a method for co-occupying the GATA binding protein 2 (Gata2) locus within a cell comprising contacting the cell with either a histone deacetylase 1 (HDAC1) inhibitor and a HDAC2 inhibitor. In some aspects, the HDAC1 inhibitor or HDAC2 inhibitor is a compound of Formula I, II, III, IV, V, VI, VII, VIII, IX, X, or any of the compounds presented in Table 1, Table 2, Table 3, or Table 4, or a pharmaceutically acceptable salt thereof.

In another aspect, provided herein is a method for hyperacetylating histones at GATA binding protein 2 (Gata2) regulatory regions within a cell comprising contacting the cell with a compound of Formula I, II, III, IV, V, VI, VII, VIII, IX, X, or any of the compounds presented in Table 1, Table 2, Table 3, or Table 4, or a pharmaceutically acceptable salt thereof.

In another aspect, provided herein is a method for increasing GATA binding protein 2 (Gata2) at the HbD (delta globin) promoter within a cell comprising contacting the cell with a compound of Formula I, II, III, IV, V, VI, VII, VIII, IX, X, or any of the compounds presented in Table 1, Table 2, Table 3, or Table 4, or a pharmaceutically acceptable salt thereof. In some aspects, increased Gata2 binding at the HbD promoter alters HbG expression.

In still another aspect, provided herein is a method for treating acute myeloid leukemia (AML) comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof.

In one aspect, provided herein is a method for treating familial myelodysplastic syndrome (MDS) comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof.

In another aspect, provided herein is a method for treating leukemia comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof.

In yet another aspect, provided herein is a method for treating sickle-cell anemia comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof.

In still another aspect, provided herein is a method for treating beta-thalassemia comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof.

In another aspect, provided herein is a method for treating monocytopenia comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof.

In yet another aspect, provided herein is a method for treating mycobacterial infections comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof.

In still another aspect, provided herein is a method for treating dendritic cell lymphoid deficiency comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof.

In another aspect, provided herein is a method for treating nonocyte lymphoid deficiency comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof.

In yet another aspect, provided herein is a method for treating B lymphoid deficiency comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof.

In still another aspect, provided herein is a method for treating natural killer lymphoid deficiency comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof.

In another aspect, provided herein is a method for treating Emberger syndrome comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof.

In yet another aspect, provided herein is a method for treating asymptomatic neurocognitive impairment comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof.

In still another aspect, provided herein is a method for treating mild neurocognitive disorder comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof.

In another aspect, provided herein is a method for treating HIV-associated dementia comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically acceptable salt thereof.

In one aspect, provided herein is a method for treating a disease or disorder associated with Gata2 deficiency comprising administering to a subject in need thereof a therapeutically effective amount of Compound 001, or a pharmaceutically acceptable salt thereof.

In another aspect, provided herein are methods for increasing Gata2 expression in a cell comprising contacting the cell with Compound 001, or a pharmaceutically acceptable salt thereof. In some aspects, Gata2 overexpression induces HbG (gamma globin).

In an additional aspect, provided herein are methods for increasing acetylation at Gata2 regulatory regions within a cell comprising contacting the cell with Compound 001, or a pharmaceutically acceptable salt thereof.

In a further aspect, provided herein are methods for increasing binding of Gata2 to Gata2 regulatory regions within a cell comprising contacting the cell with Compound 001, or a pharmaceutically acceptable salt thereof.

In another aspect, provided herein is a method for increasing Gata2 expression in a cell comprising contacting the cell with Compound 001, or a pharmaceutically acceptable salt thereof. In some aspects, Gata2 overexpression induces HbG (gamma globin).

In an additional aspect, provided herein is a method for increasing acetylation at Gata2 regulatory regions within a cell comprising contacting the cell with Compound 001, or a pharmaceutically acceptable salt thereof.

In a further aspect, provided herein is a method for increasing binding of Gata2 to Gata2 regulatory regions within a cell comprising contacting the cell with Compound 001, or a pharmaceutically acceptable salt thereof.

In one aspect, provided herein is a method of selectively inhibiting HDAC1 or HDAC2 over other HDACs in a subject comprising administering a compound of Formula I, II, II, IV, V, VI, or any of the compounds presented in Table 1, Table 2, Table 3, or Table 4, and pharmaceutically acceptable salts thereof. In some embodiments, the compound has a selectivity for HDAC1 over HDAC2. In other embodiments, the compound has a selectivity for HDAC2 over HDAC1. In some embodiments, the compound has a balanced HDAC1 and HDAC2 selectivity. The term “balanced” means that the selectivity for HDAC1 and HDAC2 is approximately equal, i.e., that the selectivities for HDAC1 and HDAC2 are within about ±10% of each other.

In another aspect, provided herein is a method for inducing histone acetylation within a cell by contacting the cell with Compound 001, or a pharmaceutically acceptable salt thereof.

In another aspect, provided herein is a method for inducing HbG (gamma globin) within a cell by contacting the cell with Compound 001, or a pharmaceutically acceptable salt thereof. In some aspects, the cell is a sickle cell.

In another aspect, provided herein is a method for inducing HbF (fetal hemoglobin) within a cell by contacting the cell with Compound 001, or a pharmaceutically acceptable salt thereof.

In another aspect, provided herein is a method for attenuating HbG (gamma globin) induction by a histone deacetylase 1 (HDAC1) inhibitor or a HDAC2 inhibitor within a cell comprising contacting the cell with a compound that knocks down GATA binding protein 2 (Gata2), wherein the compound is Compound 001, or a pharmaceutically acceptable salt thereof.

In another aspect, provided herein is a method for co-occupying the GATA binding protein 2 (Gata2) locus within a cell comprising contacting the cell with Compound 001, or a pharmaceutically acceptable salt thereof.

In another aspect, provided herein is a method for hyperacetylating histones at GATA binding protein 2 (Gata2) regulatory regions within a cell comprising contacting the cell with Compound 001, or a pharmaceutically acceptable salt thereof.

In another aspect, provided herein is a method for increasing GATA binding protein 2 (Gata2) at the HbD (delta globin) promoter within a cell comprising contacting the cell with Compound 001, or a pharmaceutically acceptable salt thereof. In some aspects, increased Gata2 binding at the HbD promoter alters HbG expression.

In still another aspect, provided herein is a method for treating acute myeloid leukemia (AML) comprising administering to a subject in need thereof a therapeutically effective amount of Compound 001, or a pharmaceutically acceptable salt thereof.

In one aspect, provided herein is a method for treating familial myelodysplastic syndrome (MDS) comprising administering to a subject in need thereof a therapeutically effective amount of Compound 001, or a pharmaceutically acceptable salt thereof.

In another aspect, provided herein is a method for treating leukemia comprising administering to a subject in need thereof a therapeutically effective amount of Compound 001, or a pharmaceutically acceptable salt thereof.

In yet another aspect, provided herein is a method for treating sickle-cell anemia comprising administering to a subject in need thereof a therapeutically effective amount of Compound 001, or a pharmaceutically acceptable salt thereof.

In still another aspect, provided herein is a method for treating beta-thalassemia comprising administering to a subject in need thereof a therapeutically effective amount of Compound 001, or a pharmaceutically acceptable salt thereof.

In another aspect, provided herein is a method for treating monocytopenia comprising administering to a subject in need thereof a therapeutically effective amount of Compound 001, or a pharmaceutically acceptable salt thereof.

In yet another aspect, provided herein is a method for treating mycobacterial infections comprising administering to a subject in need thereof a therapeutically effective amount of Compound 001, or a pharmaceutically acceptable salt thereof.

In still another aspect, provided herein is a method for treating dendritic cell lymphoid deficiency comprising administering to a subject in need thereof a therapeutically effective amount of Compound 001, or a pharmaceutically acceptable salt thereof.

In another aspect, provided herein is a method for treating nonocyte lymphoid deficiency comprising administering to a subject in need thereof a therapeutically effective amount of Compound 001, or a pharmaceutically acceptable salt thereof.

In yet another aspect, provided herein is a method for treating B lymphoid deficiency comprising administering to a subject in need thereof a therapeutically effective amount of Compound 001, or a pharmaceutically acceptable salt thereof.

In still another aspect, provided herein is a method for treating natural killer lymphoid deficiency comprising administering to a subject in need thereof a therapeutically effective amount of Compound 001, or a pharmaceutically acceptable salt thereof.

In another aspect, provided herein is a method for treating Emberger syndrome comprising administering to a subject in need thereof a therapeutically effective amount of Compound 001, or a pharmaceutically acceptable salt thereof.

In yet another aspect, provided herein is a method for treating asymptomatic neurocognitive impairment comprising administering to a subject in need thereof a therapeutically effective amount of Compound 001, or a pharmaceutically acceptable salt thereof.

In still another aspect, provided herein is a method for treating mild neurocognitive disorder comprising administering to a subject in need thereof a therapeutically effective amount of Compound 001, or a pharmaceutically acceptable salt thereof.

In another aspect, provided herein is a method for treating HIV-associated dementia comprising administering to a subject in need thereof a therapeutically effective amount of Compound 001, or a pharmaceutically acceptable salt thereof.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In certain embodiments, provided herein is a method of treatment of any of the disorders described herein, wherein the subject is a human.

In accordance with the foregoing, provided herein is a method for treating any of the diseases or disorders described above in a subject in need of such treatment, which method comprises administering to the subject a therapeutically effective amount of a compound provided herein or a pharmaceutically acceptable salt thereof. For any of the above uses, the required dosage will vary depending on the mode of administration, the particular condition to be treated and the effect desired.

In some embodiments of any of the methods provided herein, hematopoietic cytotoxicity is minimized.

Another aspect provided herein is the use of a compound as described herein (e.g., of any formulae herein) in the manufacture of a medicament for use in the treatment of a disorder or disease herein.

Another aspect provided herein is the use of a compound as described herein (e.g., of any formulae herein) for use in the treatment of a disorder or disease herein.

According to the methods provided herein, disorders are treated in a subject, such as a human or other animal, by administering to the subject a therapeutically effective amount of a compound provided herein, in such amounts and for such time as is necessary to achieve the desired result. The term “therapeutically effective amount” of a compound provided herein means a sufficient amount of the compound so as to decrease the symptoms of a disorder in a subject. As is well understood in the medical arts a therapeutically effective amount of a compound provided herein will be at a reasonable benefit/risk ratio applicable to any medical treatment.

In general, compounds provided herein will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight (0.05 to 4.5 mg/m²). An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient. In certain embodiments, intermittent dose administration is applied.

In certain embodiments, a therapeutic amount or dose of the compounds provided herein may range from about 0.1 mg/kg to about 500 mg/kg (about 0.18 mg/m² to about 900 mg/m²), alternatively from about 1 to about 50 mg/kg (about 1.8 to about 90 mg/m²). In general, treatment regimens provided herein comprise administration to a patient in need of such treatment from about 10 mg to about 1000 mg of the compound(s) provided herein per day in single or multiple doses. Therapeutic amounts or doses will also vary depending on route of administration, as well as the possibility of co-usage with other agents.

Upon improvement of a subject's condition, a maintenance dose of a compound, composition or combination provided herein may be administered, if necessary. Subsequently, the dosage or frequency of administration, or both, may be reduced, as a function of the symptoms, to a level at which the improved condition is retained. When the symptoms have been alleviated to the desired level, treatment should cease. The subject may, however, require intermittent treatment on a long-term basis upon any recurrence of disease symptoms.

It will be understood, however, that the total daily usage of the compounds and compositions provided herein will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

### EXAMPLES

Examples are set forth below for the purpose of illustration and to describe certain specific embodiments and aspects as provided herein. However, the scope of the claims is not to be in any way limited by the examples set forth herein. Various changes and modifications to the disclosed embodiments and aspects will be apparent to those skilled in the art and such changes and modifications including, without limitation, those relating to the chemical structures, substitutents, derivatives, formulations and methods provided herein may be made without departing from the spirit of the subject-matter provided herein and the scope of the appended claims. Definitions of the variables in the structures in the schemes herein are commensurate with those of corresponding positions in the formulae presented herein.

### Example 1

### Synthesis of N-(2-amino-5-(thiophen-2-yl)phenyl)-2-(piperazin-1-yl)quinoline-6-carboxamide, Compound 001

Methods of synthesizing compounds of Formulae I, II, and III (e.g., compounds of Tables 1 and 2) can be found in U.S. Patent Publication No. 2014-0128391, which is hereby incorporated by reference in its entirety.

The preparation of Compound 001 is provided in U.S. Patent Publication No. 2014-0128391 as Example 27, and is summarized below.

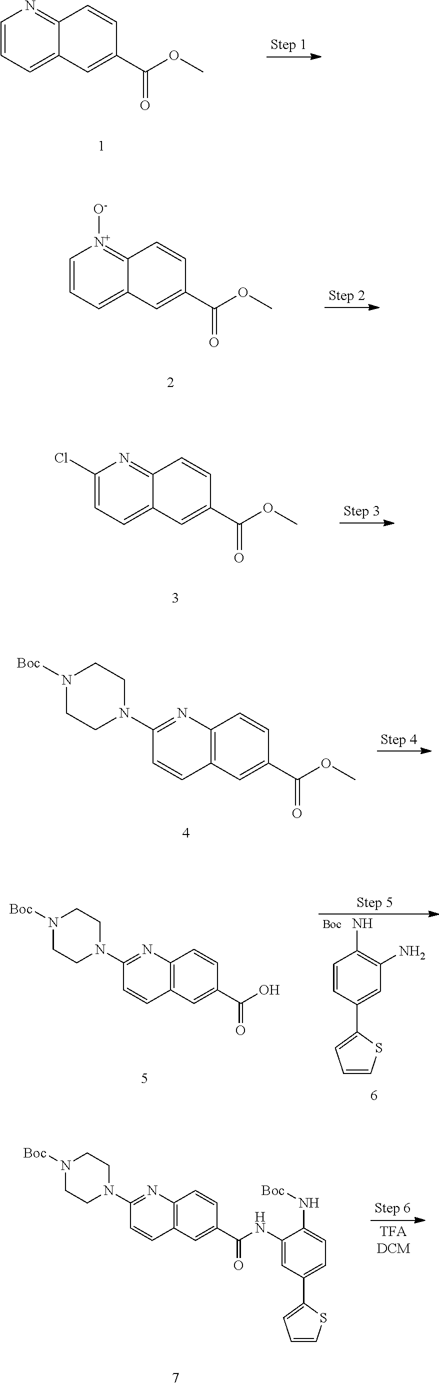

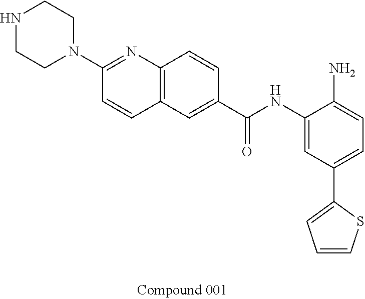 [SMILES: Nc1ccc(-c2cccs2)cc1NC(=O)c1ccc2nc(N3CCNCC3)ccc2c1]

### Experimental Procedure

Step 1: A mixture of compound 1 (10 g, 0.53 mol) and m-CPBA (18.4 g, 0.106 mol) in DCM (50 ml) is stirred at r.t. overnight. Aq. NaHCO₃ (40 ml, saturated) is added to the reaction mixture and stirred for 30 min. The organic layer is separated, dried, filtered and concentrated to obtain a residue, which can be re-crystallized in ethyl acetate (5 ml) to afford compound 2 as a light yellow solid.

Step 2: To a solution of compound 2 (4.0 g, 0.020) and DMF (8 ml) in DCM is added SOCl₂ (8 ml) slowly at 0° C. and stirred at r.t. for 5 h. The resulting mixture is concentrated to obtain a residue, and DCM (50 ml) with Aq. NaHCO₃ (saturated, 20 ml) is added and stirred for 30 min. The organic layer is separated and concentrated to obtain a residue, which is purified by silica gel chromatography to afford compound 3 as a white solid.

Step 3: A mixture of compound 3 (10 g, 0.045 mol), CuI (10 g, 0.53 mol), N-boc-piperazine (25 g, 0.135 mol) and K₂CO₃ (18.6 g, 0.135 mol) in DMSO (120 ml) is stirred at 100° C. overnight. Upon completion, as monitored by TLC (thin-layer chromatography), 300 ml of EA (ethyl acetate) is added, followed by filtration. Concentration of the mixture yields a residue, to which water (300 ml) and Aq. Citric acid (saturated, 30 ml) are added. Stirring at r.t. for 30 min., followed by filtration yields compound 4 as a yellow solid that can be used in the next step without purification.

Step 4: A mixture of compound 4 (18 g, crude) and 2M NaOH (50 ml) in EtOH (100 ml) and THF (100 ml) is stirred at 70° C. for 4 h. TLC can be used to monitor the reaction. The reaction mixture is concentrated to a residue, to which water (300 ml) and aq. sat. citric acid (40 ml) are added. Subsequent filtration yields compound 5 as a yellow solid.

Step 5: A mixture of compound 5 (1 equiv.), tert-butyl 2-amino-4-(thiophen-2-yl)phenylcarbamate (1 equiv.), HOAT (1.5 equiv.), EDCI (2 equiv.), and DIPEA (4 equiv.) in DMF is stirred at 55° C. overnight. Water is added to the mixture, and extracted with EA. The organic layers are separated, dried, filtered, and concentrated to yield a residue, which can be purified by Prep-TLC to afford compound 7.

Step 6: A mixture of compound 7 (95 mg 0.15 mmol) and TFA (2 ml) in 2 ml DCM is stirred at r.t. for 2 h. Evaporation of the solvent yields crude product which can be purified by HPLC to afford the white product, Compound 001 (19 mg, 30%). ¹H NMR (500 MHz, DMSO) δ 9.79 (s, 1H), 8.42 (d, J=1.8 Hz, 1H), 8.17-8.09 (m, 2H), 7.60 (d, J=8.8 Hz, 1H), 7.51 (d, J=2.0 Hz, 1H), 7.36 (dd, J=5.1, 0.8 Hz, 1H), 7.33-7.28 (m, 2H), 7.25 (d, J=3.5 Hz, 1H), 7.06 (dd, J=5.0, 3.6 Hz, 1H), 6.83 (d, J=8.3 Hz, 1H), 5.18 (s, 2H), 3.73 (s, 4H), 2.89 (s, 4H). LCMS: m/z=430 (M+H)⁺.

### Example 2

### Synthesis of 2-((1-acetyl-4-phenylpiperidin-4-yl)amino)-N-hydroxypyrimidine-5-carboxamide (Compound 1003)

Methods of synthesizing compounds of Formulae IV and V (e.g., compounds of Table 3) can be found in U.S. Patent Publication No. 2015-0105384, which corresponds to PCT Publication No. WO 2015/054474, each of which are incorporated herein by reference in its entirety.

The preparation of Compound 1003 is provided in WO 2015/054474 as Example 1, and is summarized below.

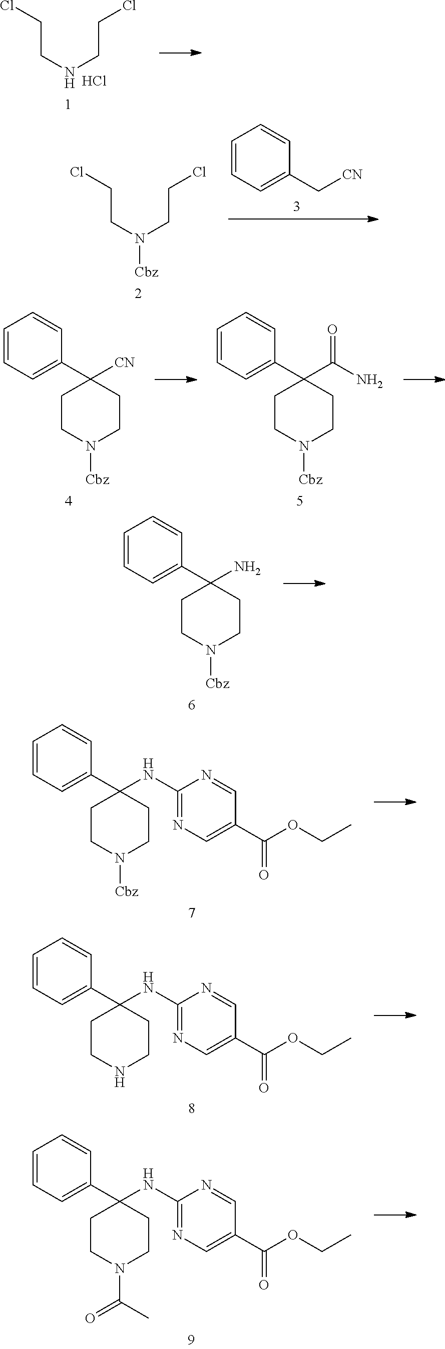

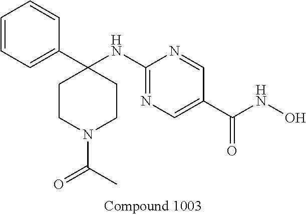 [SMILES: CC(=O)N1CCC(Nc2ncc(C(=O)NO)cn2)(c2ccccc2)CC1]

Step 1: To a solution of 1 (10.4 g, 56.5 mmol) and TEA (11.4 g, 113 mmol) in DCM (60 mL) was added dropwise CbzCl (benzyl chloroformate, 10 g, 56.5 mmol) over 30 mins at 0° C. Then the mixture was stirred at room temperature (r.t.) for 6 hrs. H₂O (50 ml) was added, the organic layer was washed with aqueous NaCl, dried by anhydrous Na₂SO₄, concentrated in vacuo and the residue was purified by silica gel chromatography (PE/EA=20:1) to afford compound 2 as a white solid (11.6 g, yield: 70%).

Step 2: To a flask containing compound 3 (1.52 g, 13.1 mmol) and compound 2 (3 g, 10.9 mol) in DMF (25 ml) was added NaH (1.09 g, 27.2 mmol) at 0° C. It was stirred at 60° C. for 3 hrs. H₂O was added, the resulting mixture was extracted with ethyl acetate (EA). The combined EA layers were concentrated in vacuo and the residue was purified by silica gel chromatography (PE/EA=2:1) to afford compound 4 as a yellow solid (1.9 g, yield: 54%).

Step 3: To a mixture of compound 4 (1.89 g, 5.91 mmol) in DMSO (15 mL) was added K₂CO₃ (2.4 g, 17.7 mmol), the mixture was stirred at 60° C. Then to the reaction 30% H₂O₂ (17 ml, 177 mmol) was added dropwise. After the reaction was complete, H₂O was added, and the reaction mixture was filtered. The resulting white solid was dried to afford compound 5 1.99 g, yield: 70%).

Step 4: A mixture of compound 5 (6.2 g, 18.3 mmol), NaClO (11 ml, 25.6 mol), and 3N NaOH (17 mL, 51.3 mmol) in t-BuOH (40 mL) was stirred at 0° C. to r.t. overnight. The mixture was concentrated, extracted with EA (30 mL×2), washed with aqueous NaCl, dried by Na₂SO₄, and concentrated to afford compound 6 (4.5 g, yield: 80%).

Step 5: To a solution of compound 6 (2.0 g, 6.45 mmol) in Dioxane (18 mL) was added ethyl 2-chloropyrimidine-5-carboxylate (1.08 g, 5.80 mmol), N,N-diisopropylethylamine (DIPEA) (1.7 g, 12.9 mmol) at 105° C. The reaction was stirred overnight. The reaction mixture was concentrated in vacuo, and the residue was purified by silica gel chromatography (PE/EA=6:1) to give compound 7 (1.5 g, yield: 51%).

Step 6: HBr/AcOH (6.0 mL) was added to a flask containing compound 7 (3.0 g, 6.52 mmol) at r.t. for 3 hrs. Then 12 ml Et₂O was added, the reaction mixture filtered, the solid was dried to give compound 8 (1.85 g, yield: 70%) as a yellow solid.

Step 7: To a solution of compound 8 (100 mg, 0.31 mmol) in DCM (4 mL) was added Ac₂O (47 mg, 0.46 mmol), and Et₃N (0.5 ml) at r.t. The reaction was stirred for 2 hrs and the reaction mixture was concentrated in vacuo to give compound 9 (120 g, yield: 100%).

Step 8: To a solution of compound 9 (20 mg, 0.33 mmol) in MeOH (2 mL) and DCM (1 ml) at 0° C. was added NH₂OH (0.4 ml) and stirred for 10 mins. Then NaOH/MeOH (0.8 ml) was added and the reaction was stirred for 2 hrs. The mixture was concentrated, adjusted to a pH=5 using 2N HCl, extracted with EA (10 ml) and purified by preparative-HPLC to afford 2-((1-acetyl-4-phenylpiperidin-4-yl)amino)-N-hydroxypyrimidine-5-carboxamide (18 mg, 16%). ¹H NMR (500 MHz, DMSO): δ 10.95 (s, 1H), 8.98 (s, 1H), 8.62 (s, 1H), 8.33 (s, 1H), 8.23 (s, 1H), 7.38 (d, J=7.6 Hz, 2H), 7.27 (t, J=7.7 Hz, 2H), 7.16 (t, J=7.3 Hz, 1H), 4.28 (d, J=13.2 Hz, 1H), 3.72 (d, J=13.6 Hz, 1H), 3.39-3.28 (m, 1H), 2.85 (t, J=12.3 Hz, 1H), 2.61 (t, J=12.5 Hz, 2H), 2.01 (s, 3H), 1.97-1.86 (m, 1H), 1.77 (t, J=11.0 Hz, 1H). LCMS: m/z=356 (M+H)⁺.

### Example 3

### Synthesis of N-hydroxy-2-((4-phenyl-1-(phenylcarbamoyl)piperidin-4-yl)amino)pyrimidine-5-carboxamide (Compound 1001)

The preparation of Compound 1001 is provided in WO 2015/054474 as Example 3, and is summarized below.

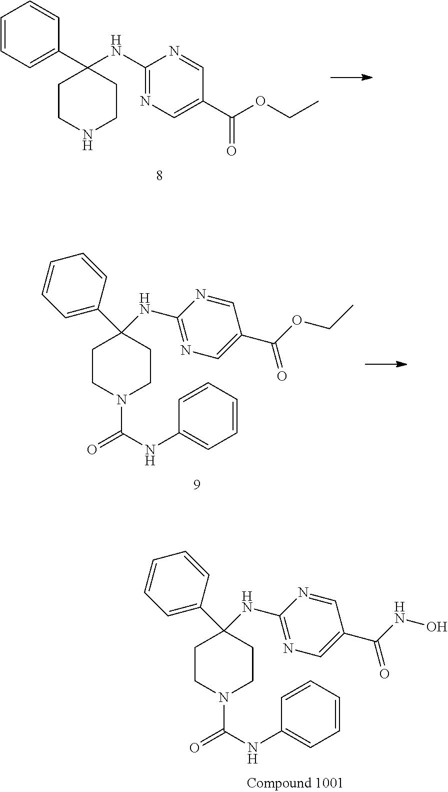 [SMILES: CCOC(=O)c1cnc(NC2(c3ccccc3)CCNCC2)nc1 ; CCOC(=O)c1cnc(NC2(c3ccccc3)CCN(C(=O)Nc3ccccc3)CC2)nc1 ; O=C(NO)c1cnc(NC2(c3ccccc3)CCN(C(=O)Nc3ccccc3)CC2)nc1]

Step 1: To a solution of compound 8 (85 mg, 0.26 mmol) in THF (4 mL) was added isocyanatobenzene (46 mg, 0.39 mmol), DIPEA (0.2 ml) at r.t. The reaction was stirred for 2 hrs. and subsequently concentrated in vacuo to give compound 9 (80 g, yield: 69%).

Step 2: To a solution of compound 9 (80 mg, 0.18 mmol) in MeOH (3 mL) and DCM (1 ml) at 0° C. was added NH₂OH (0.2 ml). The reaction was stirred for 10 mins, at which time NaOH/MeOH (0.4 ml) was added. The reaction was stirred for 2 hrs. The resulting reaction mixture was concentrated, adjusted to pH=5 using 2N HCl, extracted with EA (10 ml), and purified by Preparative-HPLC to afford N-hydroxy-2-((4-phenyl-1-(phenylcarbamoyl)piperidin-4-yl)amino)pyrimidine-5-carboxamide (14 mg, 17%). ¹H NMR (500 MHz, DMSO) δ 10.83 (s, 1H), 8.96 (s, 1H), 8.60 (s, 1H), 8.49 (s, 2H), 8.37 (s, 1H), 8.20 (s, 1H), 7.47-7.46 (d, J=7.6 Hz, 2H), 7.41-7.39 (d, J=7.4 Hz, 2H), 7.29-7.26 (t, J=7.7 Hz, 2H), 7.23-7.20 (m, J=7.7 Hz, 2H), 7.18-7.15 (t, J=7.3 Hz, 1H), 6.92 (t, J=7.3 Hz, 1H), 4.03 (d, J=13.2 Hz, 2H), 3.13 (t, J=12.1 Hz, 2H), 2.64 (d, J=13.0 Hz, 2H), 1.90 (t, J=11.0 Hz, 2H). LCMS: m/z=433 (M+H)⁺

Step 3: To a solution of compound 3 (38 g, 112 mmol) in 300 ml DMSO was added 30% H₂O₂ (190 ml, 2248 mmol) slowly at 0° C. followed by stirring for 30 mins. Then the temperature was slowly increased to 40° C. and stirred for an additional 30 mins. After increasing the temperature to 60° C., the mixture was stirred at 60° C. overnight. TLC was used to monitor the reaction to completion. After cooling, water was added into the mixture to give a white solid, which was isolated by filtration (38 g, ˜95%).

Step 4: To a solution of compound 4 (38 g, 106 mmol) in 400 ml BuOH was slowly added NaClO (64.2 ml, 149 mmol) followed by 3N NaOH (99 ml, 298 mmol) at 0° C. Then the mixture was stirred at r.t. overnight. TLC was used to monitor the reaction to completion. The mixture was concentrated and extracted with EtOAc. The organic layer was separated, washed and dried. Then the mixture was dissolved in Et₂O, and the pH was adjusted to 2 using HCl/Dioxane. The precipitate was collected, yielding the target compound 5 (38 g 100%).

Step 5: To a solution of compound 5 (9.6 g, 26 mmol), 2-Cl-pyrimidine (4.9 g, 26 mmol) in 150 ml 1,4-Dioxane was added DIPEA (7.7 g, 60 mmol). The mixture was stirred at 110° C. overnight. LCMS was used to monitor the reaction to completion. Water (50 ml) was added and the mixture was extracted with EtOAc. The combined organic extracts were washed and dried. The target compound 6 (11 g, 90%) was purified by flash chromatography with PE/EA from 30:1 to 2:1.

Step 6: To a solution of compound 6 (1 g, 2.17 mmol) in MeOH (15 mL) was added Pd/C (0.1 g, 10% wq) under N₂. The reaction was stirred under an H₂ atmosphere overnight, after which it was filtered through celite and washed with MeOH. Concentration yielded compound 7 (690 mg, 98%) as a light yellow solid.

Step 7: To a mixture of compound 7 (81 mg, 0.2 mmol) and 1-isocyanato-4-methoxybenzene (21 mg, 0.2 mmol) in THF (4 ml) was added DIPEA (46 mg, 0.36 mmol). The reaction was stirred for 1 h. at r.t., concentrated, and purified by gel chromatography (DCM:MeOH=10:1) to afford 8 (80 mg, 84%) as a white solid.

Step 8: To a solution of compound 8 (80 mg, 0.16 mmol) in MeOH (3 mL) and DCM (1 ml) at 0° C. was added NH₂OH (0.2 ml) followed by stirring for 10 min. Then NaOH/MeOH (0.4 ml) was added and the reaction was stirred for 2 h. The reaction was concentrated and the pH was adjusted to 5, after which it was extracted with EA (10 ml). Purification by preparative-HPLC afforded the desired product, Compound 1008 (15 mg, 21%). ¹H NMR (500 MHz, DMSO) δ 8.60 (s, 1H), 8.32 (s, 2H), 8.19 (s, 1H), 7.40 (d, J=7.5 Hz, 2H), 7.34 (d, J=9.0 Hz, 2H), 7.27 (t, J=7.7 Hz, 2H), 7.16 (t, J=7.3 Hz, 1H), 6.81 (d, J=9.0 Hz, 2H), 4.00 (d, J=13.4 Hz, 2H), 3.70 (s, 3H), 3.11 (t, J=12.2 Hz, 2H), 2.63 (d, J=12.3 Hz, 2H), 1.89 (t, J=11.1 Hz, 2H). LCMS: m/z=463 (M+H)⁺.

### Example 4

### Synthesis of N-(2-amino-5-(thiophen-2-yl)phenyl)-2-cyclopropyl-1-(2-morpholinoethyl)-1H-indole-5-carboxamide (Compound 005)

The preparation of Compound 005 is provided in U.S. Patent Publication No. 2014-0128391 as Example 25, and is summarized below.

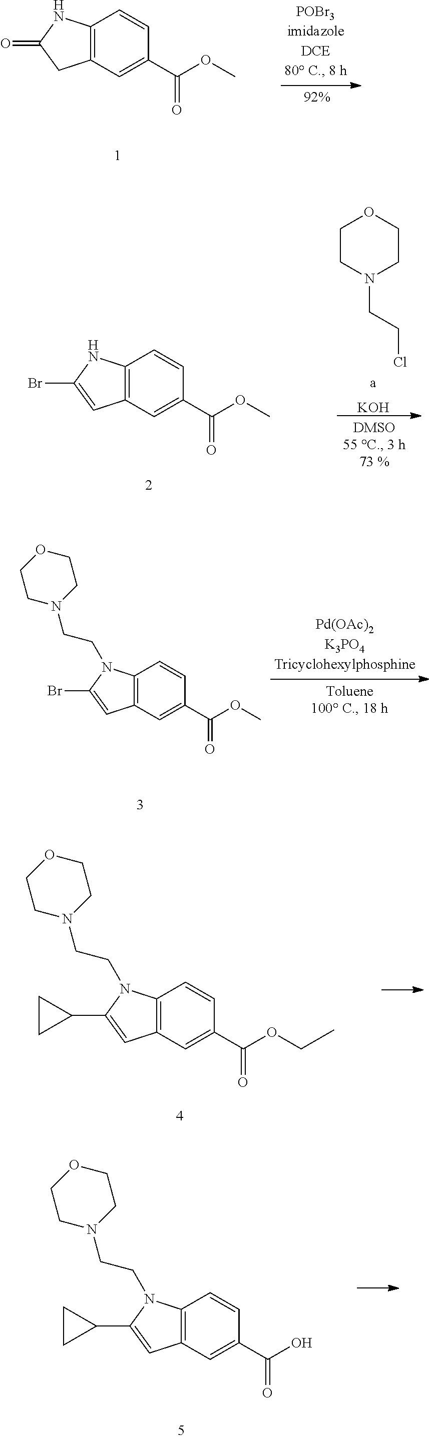

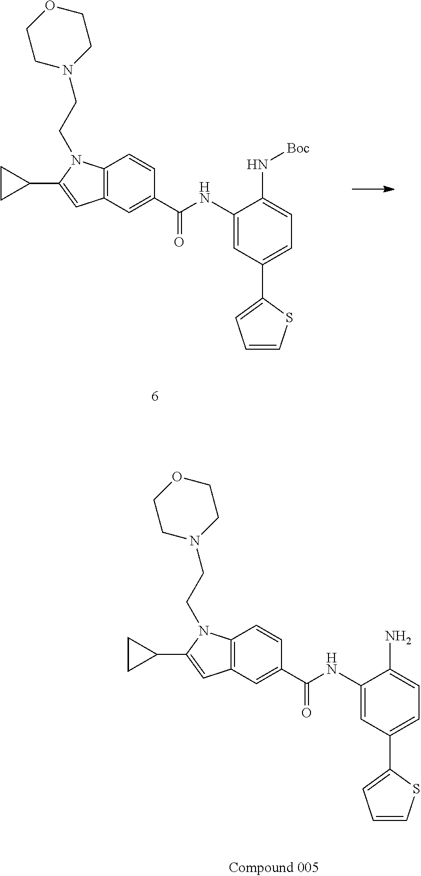 [SMILES: CC(C)(C)OC(=O)Nc1ccc(-c2cccs2)cc1NC(=O)c1ccc2c(c1)cc(C1CC1)n2CCN1CCOCC1 ; Nc1ccc(-c2cccs2)cc1NC(=O)c1ccc2c(c1)cc(C1CC1)n2CCN1CCOCC1]

### Experimental Procedure

Step 1: To a solution of compound 1 in DCE was added POBr₃ and imidazole. The reaction was stirred at 80° C. overnight. Water and DCM were added to the reaction, and the organic layer was separated, washed with brine, and dried under reduced pressure to give compound 2.

Step 2: To a solution of compound 2 in DMSO was added compound a and KOH. The resulting reaction mixture was stirred at 45° C. for 4 h, quenched with H₂O, and extracted with EA. The combined organic layers were purified by gel chromatography to yield the desired product, compound 3.

Step 3: A mixture of compound 3, cyclopropylboronic acid, Pd(OAc)₂, tricyclohexylphosphine, and K₃PO₄ in toluene and water was stirred at 100° C. under N₂ atmosphere overnight. The mixture was cooled, filtered, and concentrated to obtain a residue, which was purified by Preparative-TLC to get compound 4.

Step 4: A mixture of compound 4 and NaOH in EtOH and THF was stirred at 60° C. for 5 h. The mixture was concentrated to obtain a residue, to which was added aq. sat. citric acid and extracted with EA. The organic layers were separated, dried, filtered and concentrated to obtain compound 5.

Step 5: A mixture of compound 5, tert-butyl 2-amino-4-(thiophen-2-yl)phenylcarbamate, HOAT, EDCI, and DIPEA in DMF was stirred at 55° C. overnight. Water was added to the mixture, and extracted with EA. The organic layers were separated, dried, filtered, and concentrated to get a residue, which was purified by Preparative-TLC to afford compound 6.

Step 6: To a solution of compound 6 in DCM was added TFA and stirred at r.t. for 1 h. The mixture was concentrated to obtain a residue, which was purified by Preparative-HPLC to afford compound 005. ¹H NMR (500 MHz, DMSO) δ 9.63 (s, 1H), 8.16 (s, 1H), 7.79-7.73 (m, 1H), 7.51 (d, J=2.1 Hz, 2H), 7.36 (d, J=5.1 Hz, 1H), 7.29 (dd, J=8.3, 2.1 Hz, 1H), 7.25 (d, J=3.5 Hz, 1H), 7.05 (dd, J=5.0, 3.6 Hz, 1H), 6.82 (d, J=8.3 Hz, 1H), 6.24 (s, 1H), 5.12 (s, 2H), 4.43 (s, 2H), 3.57 (s, 5H), 2.77-2.58 (m, 2H), 2.09 (s, 1H), 1.02 (d, J=8.0 Hz, 2H), 0.76 (d, J=4.4 Hz, 2H). LCMS: m/z=487.2 (M+H)+.

### Example 5

### Synthesis of Compound Y

The preparation of Compound Y is provided in U.S. Patent Publication No. 2014-0128391 as Example 26.

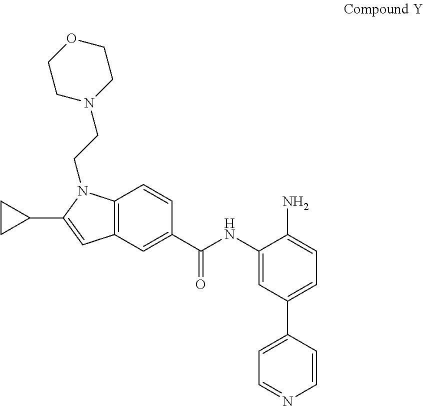 [SMILES: Nc1ccc(-c2ccncc2)cc1NC(=O)c1ccc2c(c1)cc(C1CC1)n2CCN1CCOCC1]

### Example 6

### Synthesis of Compound 2001

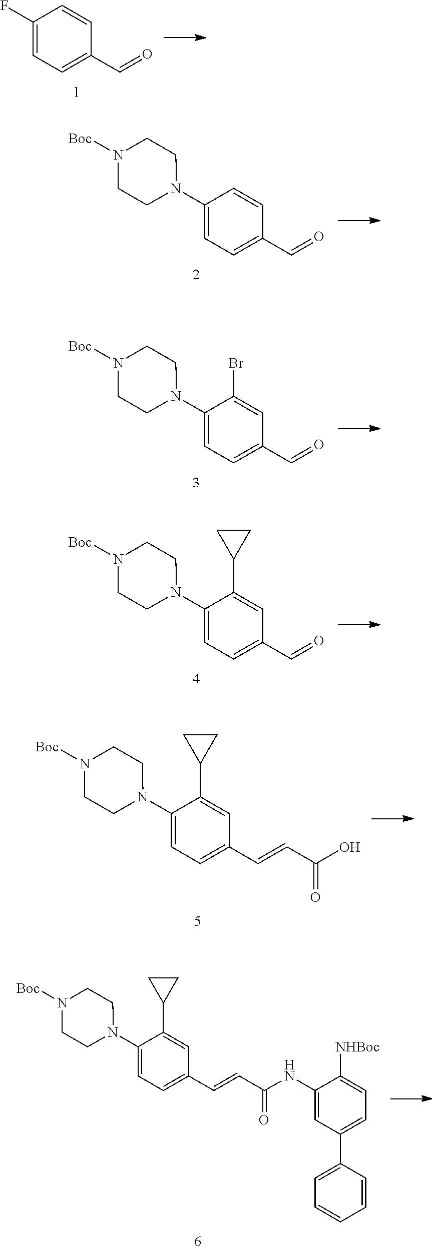 [SMILES: O=Cc1ccc(F)cc1 ; CC(C)(C)OC(=O)N1CCN(c2ccc(C=O)cc2)CC1 ; CC(C)(C)OC(=O)N1CCN(c2ccc(C=O)cc2Br)CC1 ; CC(C)(C)OC(=O)N1CCN(c2ccc(C=O)cc2C2CC2)CC1 ; CC(C)(C)OC(=O)N1CCN(c2ccc(/C=C/C(=O)O)cc2C2CC2)CC1 ; CC(C)(C)OC(=O)Nc1ccc(-c2ccccc2)cc1NC(=O)/C=C/c1ccc(N2CCN(C(=O)OC(C)(C)C)CC2)c(C2CC2)c1]

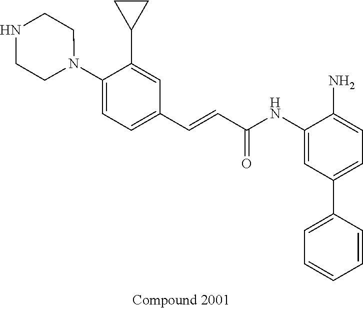 [SMILES: Nc1ccc(-c2ccccc2)cc1NC(=O)/C=C/c1ccc(N2CCNCC2)c(C2CC2)c1]

### Experimental Procedure:

Step 1: To a solution containing compound 1 (3.9 g, 31 mmol) and Boc-piperazine (6.3 g, 34 mmol) in N-methyl-2-pyrrolidone (NMP) (30 ml) was added K2CO3 (8.5 g, 62 mmol). The mixture was stirred overnight at 135° C. After completion of the reaction, the mixture was poured into ice water, and the precipitate was collected to afford the desired product as a yellow solid (6.4 g, 72%).

Step 2: To a solution of compound 2 (5.8 g, 20 mmol) in dichloromethane (DCM) (100 ml) was added N-bromosuccinimide (NBS) (3.74 g, 21 mmol). The mixture was stirred for ˜30 min at 0° C. After completion of the reaction, the mixture was directly purified by column chromatography with a mixture of petroleum ether and ethyl acetate (PE/EA) in a 5:1 ratio to afford the product (2.56 g, 35%).

Step 3: To a solution of compound 3 (1.1 g, 31 mmol), cyclopropylboronic acid (774 mg, 9 mmol), Pd(OAc)2 (67.2 mg, 0.3 mmol), tricyclohexylphosphine (TCP) (84 mg, 0.3 mmol) in toluene (6 ml)/water (6 ml) was added K3PO4 (1.9 g, 9 mmol). The mixture was refluxed overnight at 100° C. After completed, the mixture was extracted with EA (50 ml), concentrated and purified by column chromatography with a 5:1 mixture of PE/EA to afford the product as a yellow solid (1.2 g, 91%).

Step 4: A mixture of compound 4 (1.1 g, 3.3 mmol), malonic acid (1.03 g, 9.9 mmol), piperdine (841 mg, 9.9 mmol) was formed, and the mixture was heated to 100° C. overnight. The mixture was added to water and extracted with EA after using 3N HCl to adjust the solution to pH=6. The organic layer was washed with aqueous NaCl, dried over Na2SO4, concentrated, and washed with PE to afford compound 5 (1.0 g, 81%) as a yellow solid.

Step 5: To a mixture of compound 5 (90 mg, 0.24 mmol), N-(3-dimethylaminopropyl)-N′-ethylcarbodiimide (EDCI) (56 mg, 0.36 mmol), 3-hydroxytriazolo[4,5-b]pyridine (HOAT) (49 mg, 0.36 mmol), diisopropylethylamine (DIPEA) (46 mg, 0.36 mmol) and dimethylformamide (DMF) (2 mL), tert-butyl-(3-amino[1,1′-biphenyl]-4-yl)-carbamate (68.4 mg, 0.24 mmol) was added, and then the mixture was heated to 60° C. overnight. The mixture was added to water and extracted with EA. The mixture was then purified by gel chromatography (PE:EA=3:1) to afford compound 6 (80 mg, 52%) as a yellow solid.

Step 6: To a solution of compound 6 (80 mg, 0.13 mmol) in DCM (2 mL) was added trifluoroacetic acid (TFA) (0.1 mL) at 0° C., and then the reaction solution was stirred at r.t for 45 min. The mixture was concentrated, to get a residue, which was purified by preparative-HPLC to afford compound 2001 (68 mg, 95%) as a yellow solid. ¹H NMR (500 MHz, DMSO) δ 9.36 (s, 1H), 8.23 (s, 1H), 7.72 (s, 1H), 7.51 (dd, J=21.1, 11.5 Hz, 3H), 7.39 (t, J=7.8 Hz, 3H), 7.25 (dt, J=11.4, 4.7 Hz, 2H), 7.05 (d, J=8.0 Hz, 2H), 6.82 (t, J=12.7 Hz, 2H), 5.13 (s, 1H), 3.05 (s, 8H), 2.19 (d, J=5.4 Hz, 1H), 1.03 (d, J=8.2 Hz, 2H), 0.77 (d, J=4.3 Hz, 2H). LCMS: m/z=439 (M+H)⁺.

### Example 7

### Synthesis of Compound 2002

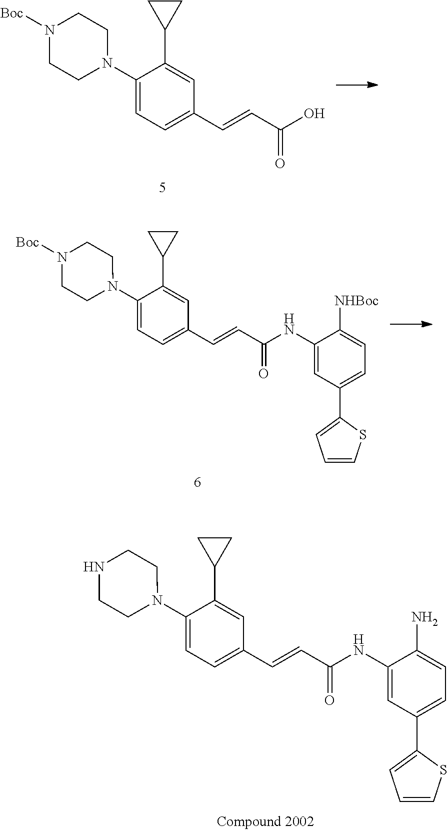 [SMILES: CC(C)(C)OC(=O)N1CCN(c2ccc(/C=C/C(=O)O)cc2C2CC2)CC1 ; CC(C)(C)OC(=O)Nc1ccc(-c2cccs2)cc1NC(=O)/C=C/c1ccc(N2CCN(C(=O)OC(C)(C)C)CC2)c(C2CC2)c1 ; Nc1ccc(-c2cccs2)cc1NC(=O)/C=C/c1ccc(N2CCNCC2)c(C2CC2)c1]

### Experimental Procedure:

Step 1: Compound 5 was prepared according to the procedure as described in Example 6, compound 5 (steps 1-4).

Step 2: A mixture of compound 5 (90 mg, 0.24 mmol), EDCI (56 mg, 0.36 mmol), HOAT (49 mg, 0.36 mmol), DIPEA (46 mg, 0.36 mmol) and DMF (2 ml) was formed, and tert-butyl-(2-amino-4-(thiophen-2-yl)phenyl)-carbamate (68.4 mg, 0.24 mmol) was added. Then the mixture was heated to 60° C. overnight. The mixture was added to water and extracted with EA. The mixture was then purified by gel chromatography (PE:EA=3:1) to afford compound 6 (90 mg, 59%) as a yellow solid.

Step 3: To a solution of compound 6 (90 mg, 0.14 mmol) in DCM (2 ml) was added TFA (0.1 ml) at 0° C., and then the reaction solution was stirred at r.t for 45 min. The mixture was concentrated, to get a residue, which was purified by preparative-HPLC to afford compound 2002 (68 mg, 90%) as a yellow solid. ¹H NMR (500 MHz, DMSO) δ 9.36 (s, 1H), 8.28 (s, 1H), 7.71 (s, 1H), 7.49 (d, J=15.6 Hz, 1H), 7.37 (dd, J=11.9, 6.5 Hz, 2H), 7.27-7.17 (m, 2H), 7.05 (dd, J=5.1, 3.6 Hz, 3H), 6.79 (t, J=11.3 Hz, 2H), 5.20 (s, 1H), 3.06 (s, 8H), 2.22-2.15 (m, 1H), 1.06-0.97 (m, 2H), 0.77 (d, J=4.6 Hz, 2H). LCMS: m/z=445 (M+H)⁺.

### Example 8

### Synthesis of Compound 2003

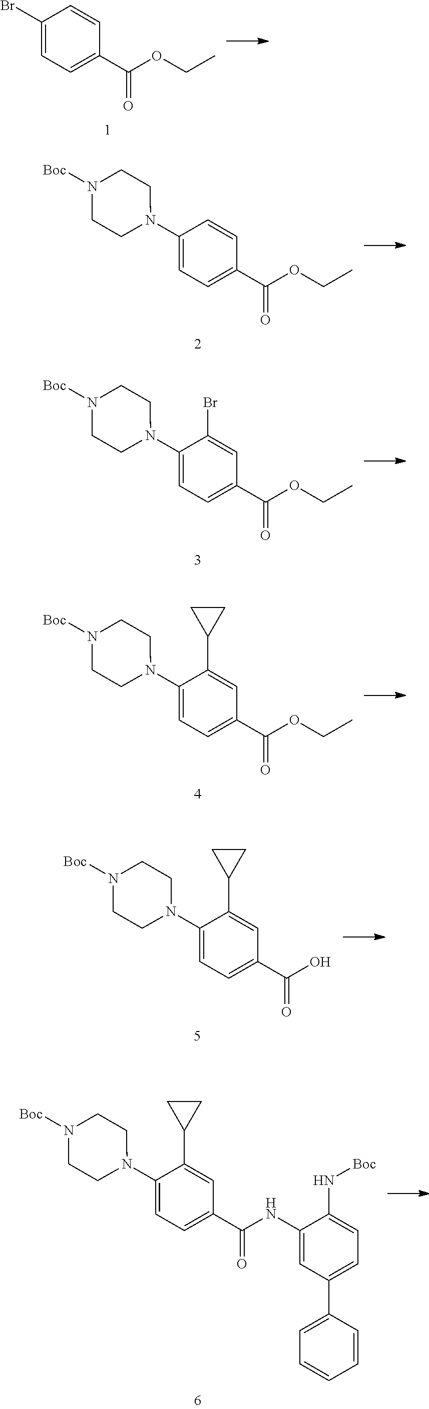 [SMILES: CCOC(=O)c1ccc(Br)cc1 ; CCOC(=O)c1ccc(N2CCN(C(=O)OC(C)(C)C)CC2)cc1 ; CCOC(=O)c1ccc(N2CCN(C(=O)OC(C)(C)C)CC2)c(Br)c1 ; CCOC(=O)c1ccc(N2CCN(C(=O)OC(C)(C)C)CC2)c(C2CC2)c1 ; CC(C)(C)OC(=O)N1CCN(c2ccc(C(=O)O)cc2C2CC2)CC1 ; CC(C)(C)OC(=O)Nc1ccc(-c2ccccc2)cc1NC(=O)c1ccc(N2CCN(C(=O)OC(C)(C)C)CC2)c(C2CC2)c1]

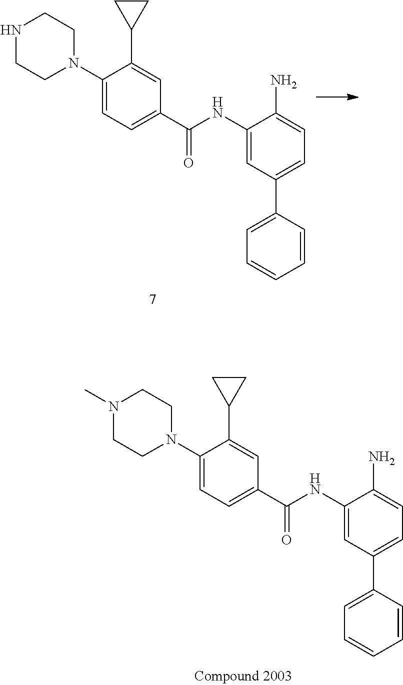 [SMILES: Nc1ccc(-c2ccccc2)cc1NC(=O)c1ccc(N2CCNCC2)c(C2CC2)c1 ; CN1CCN(c2ccc(C(=O)Nc3cc(-c4ccccc4)ccc3N)cc2C2CC2)CC1]

### Experimental Procedure:

Step 1: A mixture of compound 1 (45.6 g, 0.2 mol), Boc-piperazine (112 g, 0.6 mol), Pd2(dba)3 (18.3 g, 0.02 mol), RuPhos (9 g, 0.02 mol), Cs2CO3 (195 g, 0.6 mol) in tolune (400 mL) was stirred at 95° C. under N₂ overnight. The mixture was added to EA (200 mL), filtered and concentrated to get a residue, which was washed by PE to afford compound 2 (57 g, 87%) as a light yellow solid.

Step 2: To a solution of compound 2 (51 g, 0.15 mol) in DCM (300 mL) was added NBS (27 g, 0.15 mol) at 0° C., and the mixture was stirred for 30 min. To the mixture was added aqueous saturated Na2SO3 (50 mL) and water (200 mL), and the mixture was stirred for 30 min. The organic layer was separated, washed by water (200 ml×2), dried and concentrated to afford compound 3 (60 g, 95%) as a yellow solid.

Step 3: A mixture of compound 3 (60 g, 0.14 mol), cyclopropylboronic acid (62 g, 0.14 mol), Pd(OAc)2 (3 g, 0.014 mol), TCP (tricyclohexylphosphine, 4 g, 0.014 mol), K3PO4 (89 g, 0.42 mol) in toluene (500 ml) and water (60 ml) was stirred at 95° C. under N₂ overnight. The mixture was added to EA (200 mL), filtered, and the organic layer was separated and concentrated to get a residue, which was purified by silica gel to afford compound 4 (40 g, 74%) as a white solid.

Step 4: To a solution of compound 4 (40 g, 0.11 mol) in EtOH (200 ml) and THF (200 ml) was added NaOH (2M, 200 nil), and the mixture was stirred at 60° C. for 6 h. The mixture was concentrated to get a residue, and aqueous citric acid was added to adjust the mixture to pH<7. The solution was filtered to get compound 5 (37 g, 100%) as a white solid.

Step 5: A mixture of compound 5 (37 g, 0.11 mol), tert-butyl-(3-amino[1,1′-biphenyl]-4-yl)-carbamate (32 g, 0.11 mol), HOAT (30 g, 0.22 mol), EDCI (42 g, 0.22 mol), and NEt3 (triethylamine, 44 g, 0.44 mol) in DMF (180 ml) was stirred at 55° C. overnight. The mixture was added to water (400 ml) and extracted with EA (300 ml×2). The organic layer was separated, dried, filtered and concentrated to get a residue, which was purified by silica gel chromatography to afford compound 6 (50 g, 75%) as a white solid.

Step 6: To a solution of compound 6 (40 g, 0.064 mol) in DCM (200 ml) was added TFA (100 ml), and the mixture was stirred at r.t for 1 h. The mixture was concentrated to get a residue, to which was added EA (300 ml) and NaOH (2M, 300 ml), and the mixture was stirred for 30 min at 0° C. Then, the organic layer was separated, washed by water (200 ml×2), dried and concentrated to get compound 7 (23 g, 85%) as a white solid.

Step 7: To a solution of compound 7 (100 mg, 0.24 mmol) in DCM (5 ml) was added DIPEA (2.0 eq) and MeI (methyl iodide, 1.1 eq). The mixture was stirred at rt for 2-3 h. After completion of the reaction, the mixture was purified by preparative-HPLC to afford compound 2003 (20 mg, 20%) as a white solid. ¹H NMR (500 MHz, DMSO) δ 9.65 (s, 1H), 7.76 (d, J=0.5 Hz, 1H), 7.56 (d, J=7.5 Hz, 2H), 7.47 (s, 1H), 7.41-7.37 (m, 3H), 7.32 (d, 1H), 7.24 (t, J=7.5 Hz, 1H), 7.09 (d, J=8.5 Hz, 1H), 6.86 (d, J=8.5 Hz, 1H), 5.05 (s, 2H), 3.04 (s, 4H), 2.26 (s, 3H), 2.21-2.17 (m, 1H), 1.02 (d, J=4.5 Hz, 2H), 0.83 (d, 2H). LCMS: m/z=427 (M+H)⁺.

### Example 9

### Synthesis of Compound 2004

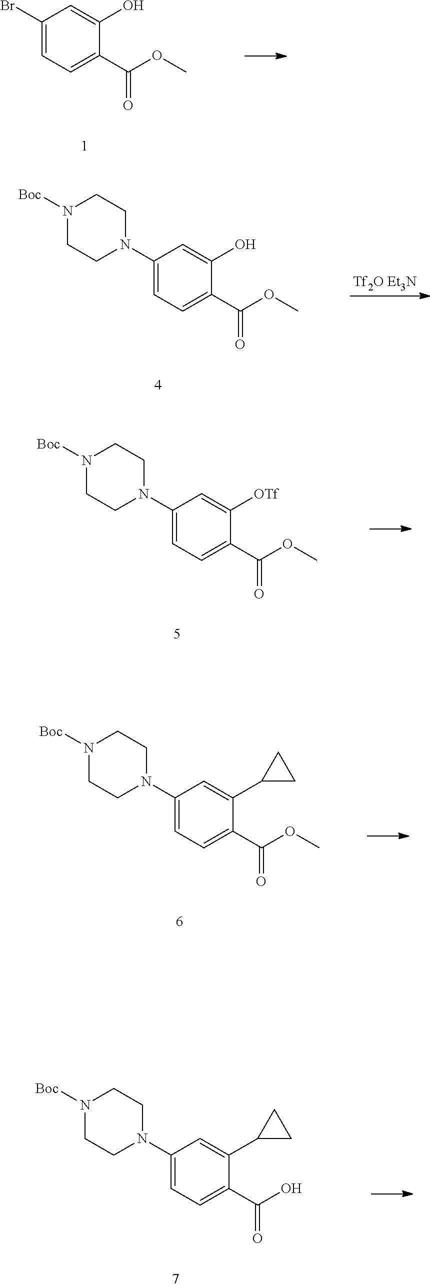 [SMILES: COC(=O)c1ccc(Br)cc1O ; COC(=O)c1ccc(N2CCN(C(=O)OC(C)(C)C)CC2)cc1O ; COC(=O)c1ccc(N2CCN(C(=O)OC(C)(C)C)CC2)cc1OS(=O)(=O)C(F)(F)F ; COC(=O)c1ccc(N2CCN(C(=O)OC(C)(C)C)CC2)cc1C1CC1 ; CC(C)(C)OC(=O)N1CCN(c2ccc(C(=O)O)c(C3CC3)c2)CC1]

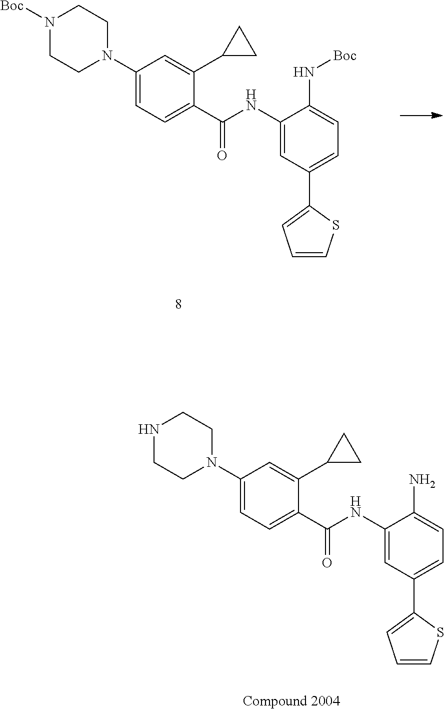 [SMILES: CC(C)(C)OC(=O)Nc1ccc(-c2cccs2)cc1NC(=O)c1ccc(N2CCN(C(=O)OC(C)(C)C)CC2)cc1C1CC1 ; Nc1ccc(-c2cccs2)cc1NC(=O)c1ccc(N2CCNCC2)cc1C1CC1]

### Experimental Procedure:

Step 1: To a solution of compound 1 (1.5 g, 6.5 mmol) and Boc-piperazine (3.6 g, 19.4 mmol) in toluene (30 ml) was added Pd2(dba)3 (0.6 g, 0.65 mmol), RuPhos (0.3 g, 0.65 mmol), and Cs2CO3 (8.4 g, 25.9 mmol). The mixture was stirred overnight at 98° C. under N₂ atmosphere. After completion of the reaction, the mixture was filtered, and then extracted by EA, washed with PE, the solvent was evaporated off to afford the target compound 4 as a yellow solid (2 g, 80%).

Step 2: To a solution of compound 4 (650 mg, 2.0 mmol) in DCM (15 ml) was added DIPEA (998 mg, 7.7 mmol), Tf2O (trifluoromethanesulfonic anhydride, 1.1 g, 3.8 mmol). The mixture was stirred for 2 h at 0° C. After completion of the reaction, the solution was concentrated to afford the product compound 5 (650 mg, 73%).

Step 3: A solution of compound 5 (650 mg, 1.4 mmol), cyclopropylboronic acid (477 mg, 5.6 mmol), Pd(dppf)₂Cl₂ (101 mg, 0.14 mmol), KF (322 mg, 5.6 mmol) in toluene (6 ml)/water (6 ml) was refluxed overnight at 100° C. under N₂ atmosphere. After completion of the reaction, the mixture was extracted with EA (50 ml), and concentrated to afford the product as a yellow solid 6 (300 mg, 60%).

Step 4: A mixture of compound 6 (300 mg, 0.83 mmol), in 2N NaOH was stirred at 65° C. for 2 h. The mixture was added to water and extracted with EA after using 3N HCl to adjust the mixture to pH=6. The mixture was washed with aqueous NaCl, dried over Na2SO4, concentrated, and washed with PE to afford compound 7 (250 mg, 87%) as a yellow solid.

Step 5: A mixture of compound 7 (100 mg, 0.29 mmol), Ph₃P (151 mg, 0.58 mmol), CBr4 (191 mg, 0.58 mmol), DIPEA (150 mg, 1.1 mmol) and DMF (2 ml) was formed, and tert-butyl-(2-amino-4-(thiophen-2-yl)phenyl)-carbamate (82 mg, 0.29 mmol) in 2 ml DMF was added. The mixture was stirred at 60° C. overnight. The mixture was added to water and was extracted with EA. The organic layer was concentrated to afford compound 8 (100 mg, crude) as a yellow solid.

Step 6: To a solution of compound 8 (100 mg, crude) in DCM (2 ml) was added TFA (0.1 ml) at 0° C., and then the reaction solution was stirred at r.t for 45 min. The mixture was concentrated, to get a residue, which was purified by preparative-HPLC to afford compound 2004 (48 mg, 37% 2 steps). ¹H NMR (500 MHz, DMSO) δ 9.64 (s, 1H), 8.92 (s, 2H), 7.65 (s, 1H), 7.49 (d, J=8.0 Hz, 1H), 7.40 (d, J=4.5 Hz, 1H), 7.32 (d, J=8.0 Hz, 1H), 7.27 (s, 1H), 7.07 (t, J=4.5 Hz, 1H), 6.90 (d, J=8.0 Hz, 1H), 6.87 (d, J=8.0 Hz, 1H), 6.53 (s, 1H), 3.42 (s, 4H), 3.23 (s, 4H), 2.40 (s, 1H), 0.913 (d, J=2.0 Hz, 2H), 0.74 (d, J=2.0 Hz, 3H). LCMS: m/z=419 (M+H)⁺.

### Example 10

### Synthesis of Compound 2005

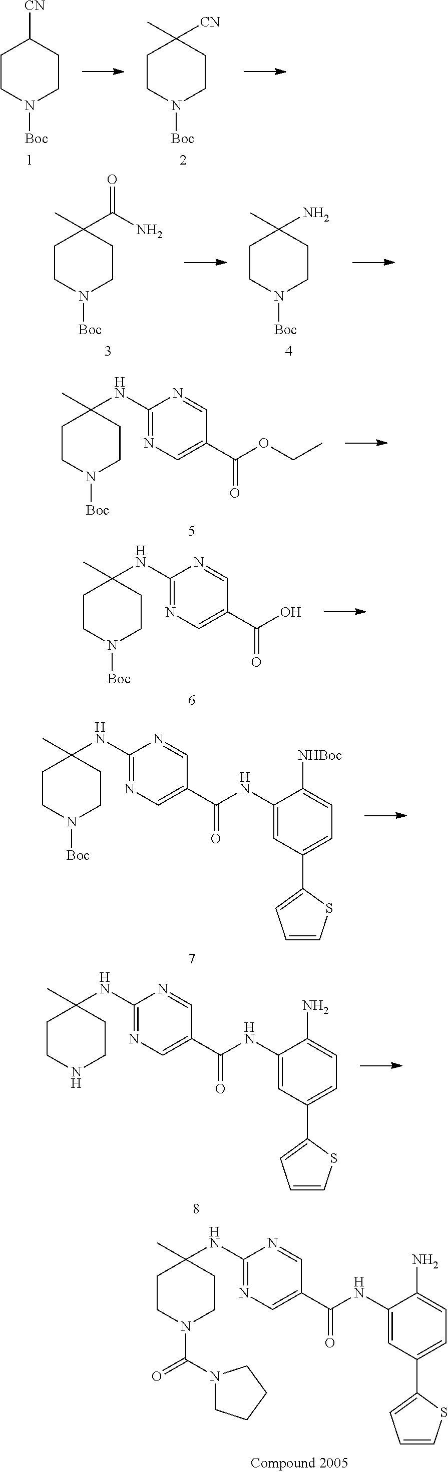

### Experimental Procedure:

Step 1: Added the lithium bis(trimethylsilyl)amide (1.0 M solution in THF, 240 mL, 240 mmol) into a round-bottomed flask containing compound 1 (25 g, 120 mmol) slowly at −76° C. under N₂. The mixture was stirred for 4 h at −76° C. Then the iodomethane (15 mL, 240 mmol) was injected into the system. The reaction mixture was stirred at −76° C. for 30 min and then was warmed to room temperature and continued stirring overnight. The reaction mixture was quenched with 150 mL saturated aqueous NH4Cl, diluted with water and extracted with ethyl acetate (EA). The organic layers were washed with water and brine then dried over sodium sulfate, filtered and concentrated to get target, compound 2, (25 g, 93%) as a light yellow solid.

Step 2: Added K2CO3 (31 g, 224 mmol) into the solution of compound 2 (25 g, 111 mmol) in DMSO (120 ml). Slowly added H2O2 (100 ml) dropwise into the system at 60° C. The reaction was stirred overnight at 60° C. The reaction mixture was poured into cold water, and the product was extracted with EA. The organic layers were washed with water and brine then dried over sodium sulfate, filtered and concentrated to get target, compound 3, (26 g, 96%) as a white solid.

Step 3: Dissolved the compound 3 (26 g, 107 mmol) in acetonitrile (200 ml) and 5N KOH (100 nil). Then added 1,3-dibromo-5,5-dimethylimidazolidine-2,4-dione (15 g, 54 mmol) into the system. The mixture was stirred overnight. Concentrated to remove acetonitrile, adjusted the pH of the water phase to 5 with 2N HCl while cooling the water phase in ice bath, extracted with EA and collected the organic layer. Then, the pH of water phase was adjusted to 10, and a white precipitate formed. The white solid as compound 4 was isolated by filtration (16 g, 69%).

Step 4: A solution of compound 4 (2 g, 9.34 mmol), ethyl 2-chloropyrimidine-5-carboxylate (2.6 g, 14.2 mmol) and DIPEA (5.3 g, 28.03 mmol) was heated in 1,4-dioxane (25 mL) at 95° C. overnight. The reaction mixture was concentrated and purified by silica gel column with EA/PE=1/5 to get compound 5 (1.8 g, 53%) as a light yellow solid.

Step 5: A solution of compound 5 (465 mg, 1.28 mmol) and 2N NaOH (10 ml, 20 mmol) in THF (10 mL) and EtOH (10 mL) was heated at 55° C. for 2 h. The reaction mixture was concentrated, and the pH of the water phase was adjusted to about pH 5 to 6. The water phase was extracted with EA. The organic layers were washed with water and brine then dried over sodium sulfate, filtered and concentrated to get target, compound 6, (400 mg, 93%) as a white solid.

Step 6: The mixture of the compound 6 (400 mg, 1.19 mmol), tert-butyl (2-amino-4-(thiophen-2-yl)phenyl)carbamate (345 mg, 1.19 mmol), EDCI (307 mg, 2.38 mmol) and DMAP (290 mg, 2.38 mmol) in DMF (10 mL) was heated at 55° C. overnight. The mixture was diluted with water and extracted with EA. The organic layers were washed with water and brine then dried over sodium sulfate, filtered and concentrated. Then the material was purified by silica gel column with EA/PE=1/2 to get compound 7 (400 mg, 55%) as a purple solid.

Step 7: A solution of compound 7 (400 mg, 0.65 mmol) was stirred with HCl/1,4-dioxane (5 mL, 20 mmol) in 1,4-dioxane (10 mL) at room temperature overnight. The reaction mixture was concentrated and washed with PE to get target compound 8 (350 mg, 100%) as a gray solid.

Step 8: Compound 8 (95 mg, 0.21 mmol) was dissolved in Et3N (106 mg, 1.05 mmol) and THF (5 ml). Pyrrolidine-1-carbonyl chloride (40 mg, 0.3 mmol) was added into the reaction mixture. The mixture was stirred at room temperature for 2 h. The reaction mixture was filtered through silica gel and washed with EA. The mixture was concentrated and purified by preparative-HPLC to get compound 2005 (19 mg, 17.5%). ¹H NMR (500 MHz, DMSO) δ 9.65 (s, 1H), 8.85 (s, 2H), 7.57 (s, 1H), 7.48 (s, 1H), 7.39 (d, J=5.1 Hz, 1H), 7.35 (d, J=8.2 Hz, 1H), 7.28 (d, J=3.0 Hz, 1H), 7.09-7.04 (m, 1H), 6.88 (d, J=8.2 Hz, 1H), 3.31 (d, J=13.3 Hz, 2H), 3.25 (s, 4H), 3.03 (t, J=10.9 Hz, 2H), 2.29 (d, J=14.2 Hz, 2H), 1.73 (s, 4H), 1.56 (s, 2H), 1.43 (s, 3H). LCMS: m/z=506 (M+H)⁺.

### Example 11

### Synthesis of Compound 2006

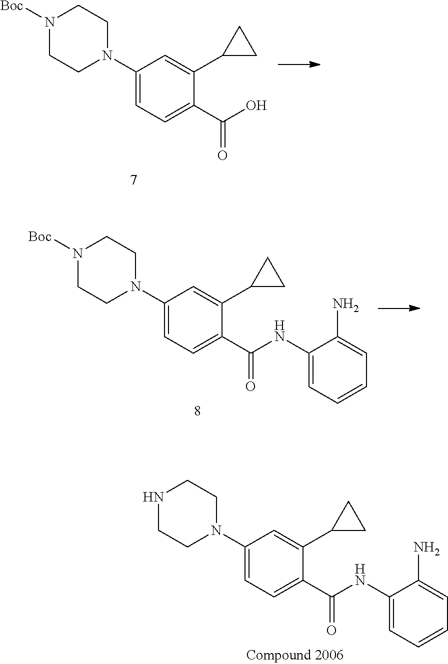 [SMILES: CC(C)(C)OC(=O)N1CCN(c2ccc(C(=O)O)c(C3CC3)c2)CC1 ; CC(C)(C)OC(=O)N1CCN(c2ccc(C(=O)Nc3ccccc3N)c(C3CC3)c2)CC1 ; Nc1ccccc1NC(=O)c1ccc(N2CCNCC2)cc1C1CC1]

### Experimental Procedure:

Step 1: To a mixture of compound 7 (100 mg, 0.29 mmol), HOAT (2.0 eq), EDCI (2.0 eq), DIPEA (2.0 eq) in DMF (5 ml) was added o-phenylenediamine (1.0 eq) in 2 ml DMF. The mixture was stirred at 60° C. overnight. To the mixture was added water, and the water phase was extracted with EA, which was concentrated to afford compound 8 (100 mg, crude) as a yellow solid.

Step 2: To a solution of compound 8 (100 mg, crude) in DCM (2 ml) was added TFA (0.1 ml) at 0° C., and then the reaction solution was stirred at r.t for 45 min. The mixture was concentrated, to get a residue, which was purified by preparative-HPLC to afford compound 2006 (35 mg, 36% 2 steps). ¹H NMR (500 MHz, DMSO) δ 9.70 (s, 1H), 8.81 (s, 2H), 7.40 (d, J=63.8 Hz, 3H), 7.10-6.74 (m, 5H), 6.52 (s, 1H), 3.39 (s, 5H), 3.23 (s, 5H), 2.37 (s, 2H), 0.90 (s, 2H), 0.73 (s, 3H). LCMS: m/z=337 (M+H)⁺.

### Example 12

### Synthesis of Compound 2007

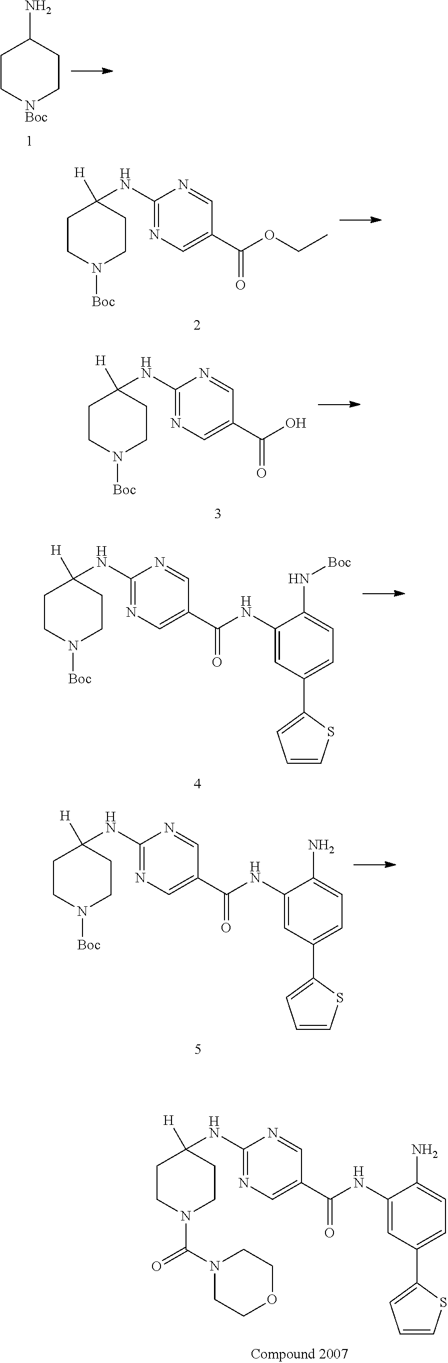 [SMILES: CC(C)(C)OC(=O)N1CCC(N)CC1 ; CCOC(=O)c1cnc(NC2CCN(C(=O)OC(C)(C)C)CC2)nc1 ; CC(C)(C)OC(=O)N1CCC(Nc2ncc(C(=O)O)cn2)CC1 ; CC(C)(C)OC(=O)Nc1ccc(-c2cccs2)cc1NC(=O)c1cnc(NC2CCN(C(=O)OC(C)(C)C)CC2)nc1 ; CC(C)(C)OC(=O)N1CCC(Nc2ncc(C(=O)Nc3cc(-c4cccs4)ccc3N)cn2)CC1 ; Nc1ccc(-c2cccs2)cc1NC(=O)c1cnc(NC2CCN(C(=O)N3CCOCC3)CC2)nc1]

### Experimental Procedure:

Step 1: A mixture of ethyl 2-chloropyrimidine-5-carboxylate (1.86 g, 10 mmol), compound 1 (4-amino-1-Boc-piperidine, 3.00 g, 15 mmol), and NEt3 (3.0 g, 30 mmol) in 1,4-dioxane (20 mL) was stirred at 95° C. overnight. The mixture was concentrated, and EA (60 mL) and aqueous citric acid (60 mL) were added to the mixture followed by stirring the mixture for 30 min. The organic layer was collected, dried and concentrated to get compound 2 (3.4 g, yield: 97%) as a light yellow solid.

Step 2: A mixture of compound 2 (3.5 g, 10 mmol) and NaOH (2M) (15 mL) in EtOH (15 mL) and THF (15 mL) was stirred at 60° C. for 2 h. The mixture was concentrated, and aqueous citric acid was added to adjust the mixture to pH<7. The mixture was stirred for 30 min and filtered to get compound 3 (2.8 g, yield: 90%) as a light yellow solid.

Step 3: A mixture of compound 3 (3.2 g, 10 mmol), tert-butyl (2-amino-4-(thiophen-2-yl)phenyl)carbamate (2.9 g, 10 mmol), HOAT (2.0 g, 15 mmol), and EDCI (3.8 g, 20 mmol) in DMF (25 mL) was stirred at 60° C. overnight. To the mixture was added EA (100 mL) and aqueous saturated NaCl (100 mL), and the mixture was stirred for 30 min. The organic layer was separated, washed by aqueous saturated NaCl (50 mL×2), dried and concentrated to get a residue, which was washed by CH3CN (about 10 to 20 mL) to get compound 4 (2.9 g, 50%) as a gray solid.

Step 4: To a solution of compound 4 (2.9 g, 5 mmol) in DCM (30 mL) was added TFA (5 mL) at rt for 2 h. The mixture was concentrated to get compound 5 (2.9 g, crude, 73%).

Step 5: To a solution of compound 5 (197 mg, 0.5 mmol) and NEt3 (250 mg, 2.5 mmol) in DCM (5 mL) was added compound morpholine-4-carbonyl chloride (97 mg, 0.65 mmol) at 0° C. LCMS was monitored to determine when the reaction was complete. To the mixture was added NH3H2O (0.5 mL), and the mixture was stirred for 30 min. The mixture was concentrated to get a residue, which was purified by silica gel column to get compound 2007 (114 mg, 45%) as a light yellow solid. ¹H NMR (500 MHz, DMSO) δ 9.54 (s, 1H), 8.86 (s, 2H), 7.88 (d, J=8.0 Hz, 1H), 7.45 (s, 1H), 7.36 (d, J=5.1 Hz, 1H), 7.31 (d, J=1.5 Hz, 1H), 7.29 (d, J=2.0 Hz, 1H), 7.25 (d, J=3.5 Hz, 1H), 7.06-7.04 (m, 1H), 6.80 (d, J=8.0 Hz, 1H), 5.21 (s, 2H), 4.02 (m, 1H), 3.63-3.57 (m, 6H), 3.13 (t, J=4.5 Hz, 4H), 2.89 (t, J=12.0 Hz, 2H). LCMS: m/z=508 (M+H)⁺.

### Example 13

### Synthesis of Compound 2008

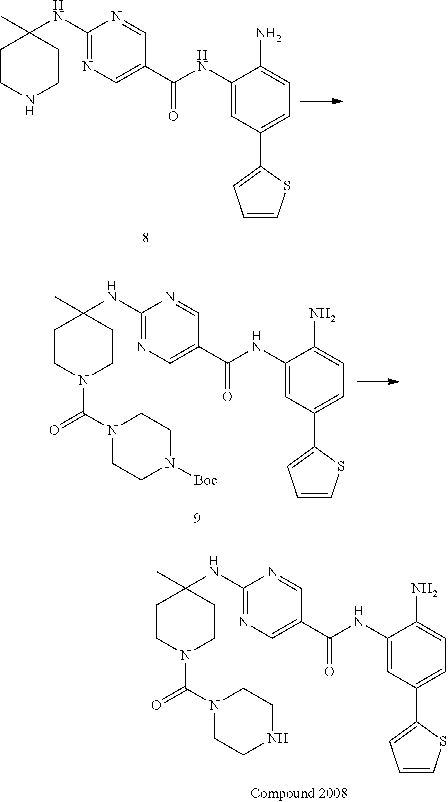 [SMILES: CC1(Nc2ncc(C(=O)Nc3cc(-c4cccs4)ccc3N)cn2)CCNCC1 ; CC1(Nc2ncc(C(=O)Nc3cc(-c4cccs4)ccc3N)cn2)CCN(C(=O)N2CCN(C(=O)OC(C)(C)C)CC2)CC1 ; CC1(Nc2ncc(C(=O)Nc3cc(-c4cccs4)ccc3N)cn2)CCN(C(=O)N2CCNCC2)CC1]

### Experimental Procedure:

Step 1: To a solution of compound 8 (115 mg, 0.28 mmol) and tert-butyl 4-(chlorocarbonyl)piperazine-1-carboxylate hydrochloride (95 mg, 0.33 mmol) was added triethylamine (85 mg, 0.84 mmol) at 0° C. The reaction was stirred at 0° C. for 2 h. Then the reaction mixture was filtered through silica gel and washed with EA. The collected EA was concentrated to get compound 9 (150 mg, 86%).

Step 2: To a solution of the compound 9 (150 mg, 0.24 mmol) in DCM (5 mL) was added TFA (4 mL). The solution was stirred at r.t. for 30 min. The reaction mixture was concentrated and purified by preparative-HPLC to get compound 2008 (88 mg, 70%) as a creamy solid. ¹H NMR (500 MHz, DMSO) δ 9.70 (s, 1H), 8.86 (d, J=4.0 Hz, 2H), 7.76 (d, J=11.0 Hz, 2H), 7.66 (d, J=3 Hz, 1H), 7.57 (d, J=7.5 Hz, 2H), 7.47 (d, J=13.0 Hz, 1H), 6.88 (d, J=11.0 Hz, 1H), 6.79 (d, J=11.5 Hz, 2H), 5.87 (s, 1H), 5.29 (s, 2H), 3.56 (t, 4H), 3.33 (s, 2H), 3.13-3.10 (m, 6H), 1.98 (d, J=17.5 Hz, 2H), 1.60 (m, 2H), 1.36 (s, 3H). LCMS: m/z=521 (M+H)⁺.

### Example 14

### Synthesis of Compound 2009

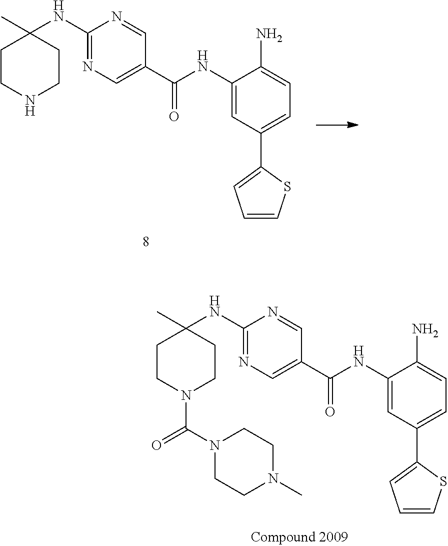 [SMILES: CC1(Nc2ncc(C(=O)Nc3cc(-c4cccs4)ccc3N)cn2)CCNCC1 ; CN1CCN(C(=O)N2CCC(C)(Nc3ncc(C(=O)Nc4cc(-c5cccs5)ccc4N)cn3)CC2)CC1]

### Experimental Procedure:

Step 1: To a solution of compound 8 (300 mg, 0.73 mmol) and 4-methylpiperazine-1-carbonyl chloride hydrochloride (175 mg, 0.87 mmol) was added triethylamine (222 mg, 2.2 mmol) at 0° C. The reaction was stirred at 0° C. for 2 h. Then, the reaction mixture was filtered through silica gel. The reaction mixture was concentrated and purified by preparative-HPLC to get compound 2009 (136 mg, 35%). ¹H NMR (500 MHz, DMSO) δ 9.72 (s, 1H), 8.86 (s, 2H), 7.64 (s, 1H), 7.49 (s, 1H), 7.41 (d, J=5.0 Hz, 1H), 7.37 (d, J=8.2 Hz, 1H), 7.30 (d, J=3.0 Hz, 1H), 7.09-7.07 (m, 1H), 6.92 (d, J=8.2 Hz, 1H), 3.62 (d, J=13.3 Hz, 2H), 3.37 (s, 4H), 3.03 (t, J=10.9 Hz, 2H), 2.81 (s, 3H), 2.3 (d, 4H), 1.56 (s, 2H), 1.43 (s, 3H). LCMS: m/z=521 (M+H)⁺.

### Example 15

### Synthesis of Compound 2010

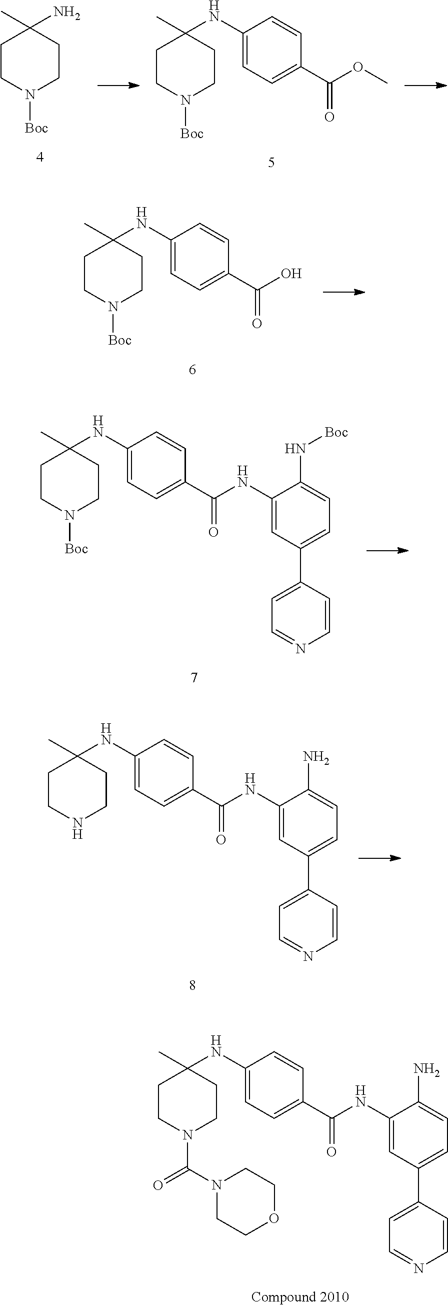 [SMILES: CC1(N)CCN(C(=O)OC(C)(C)C)CC1 ; COC(=O)c1ccc(NC2(C)CCN(C(=O)OC(C)(C)C)CC2)cc1 ; CC1(Nc2ccc(C(=O)O)cc2)CCN(C(=O)OC(C)(C)C)CC1 ; CC1(Nc2ccc(C(=O)Nc3cc(-c4ccncc4)ccc3NC(=O)OC(C)(C)C)cc2)CCN(C(=O)OC(C)(C)C)CC1 ; CC1(Nc2ccc(C(=O)Nc3cc(-c4ccncc4)ccc3N)cc2)CCNCC1 ; CC1(Nc2ccc(C(=O)Nc3cc(-c4ccncc4)ccc3N)cc2)CCN(C(=O)N2CCOCC2)CC1]

### Experimental Procedure:

Step 1: To a solution of compound 4 (600 mg, 2.8 mmol) and methyl 4-bromobenzoate (720 mg, 3.3 mmol) in toluene (10 mL) was added Pd2(dba)3 (130 mg, 0.14 mmol), RuPhos (2-dicyclohexylphosphino-2′,6′-diisopropoxybiphenyl, 130 mg, 0.28 mmol) and Cs2CO3 (273 mg, 0.84 mmol). The reaction mixture was stirred at 95° C. under N₂ overnight. The reaction mixture was concentrated to remove the solvent. Then the mixture was dissolved with water and extracted with EA. The organic phase was washed with brine and dried over Na2SO4. The organic phase was concentrated and purified by silica gel column to get compound 5 (675 mg, 79%).

Step 2: Compound 5 (675 mg, 1.94 mmol) was dissolved in EtOH (5 ml) and THF (5 ml), then 2N NaOH (5 ml) was added into the solution. The reaction was stirred at 55° C. for 1 h. The reaction mixture was concentrated to remove the solvent, and after adjusting the pH of the mixture to about 4 to 5, the aqueous phase was extracted with EA. The organic phase was washed with brine and dried over Na2SO4. The organic phase was concentrated to get compound 6 (640 mg, 99%) as a white solid.

Step 3: To a solution of the compound 6 (640 mg, 1.91 mmol) and tert-butyl (2-amino-4-(pyridin-4-yl)phenyl)carbamate (545 mg, 1.91 mmol) in DMF (10 mL) was added HOAT (520 mg, 3.83 mmol), EDCI (735 mg, 3.83 mmol) and DIPEA (740 mg, 5.73 mmol). The reaction was stirred at 55° C. overnight. The reaction was quenched with water and extracted with EA. The organic phase was washed with brine and dried over Na2SO4. The organic phase was purified by preparative-TLC to get compound 7 (475 mg, 41%) as a light yellow solid.

Step 4: To a solution of the compound 7 (250 mg, 0.41 mmol) in DCM (5 mL) was added TFA (3 mL). The solution was stirred at r.t. for 30 min. The mixture was concentrated to get compound 8 (167 mg, 100%) as a gray solid.

Step 5: To a solution the compound 8 (90 mg, 0.22 mmol) and morpholine-4-carbonyl chloride (37 mg, 0.25 mmol) was added triethylamine (67 mg, 0.66 mmol) at 0° C. The reaction was stirred at 0° C. for 2 h. Then, the reaction mixture was filtered through silica gel. The mixture was concentrated and purified by preparative-HPLC to get compound 2010 (44 mg, 38%) as a creamy solid. ¹H NMR (500 MHz, DMSO) δ 9.36 (s, 1H), 8.51 (d, J=4.0 Hz, 2H), 7.76 (d, J=11.0 Hz, 2H), 7.66 (d, J=3.0 Hz, 1H), 7.57 (d, J=7.5 Hz, 2H), 7.47 (d, J=13.0 Hz, 1H), 6.88 (d, J=11.0 Hz, 1H), 6.79 (d, J=11.5 Hz, 2H), 5.87 (s, 1H), 5.29 (s, 2H), 3.56 (t, 4H), 3.33 (s, 2H), 3.13-3.10 (m, 6H), 1.98 (d, J=17.5 Hz, 2H), 1.60 (m, 2H), 1.36 (s, 3H). LCMS: m/z=515 (M+H)⁺.

### Example 16

### Synthesis of Compound 2011

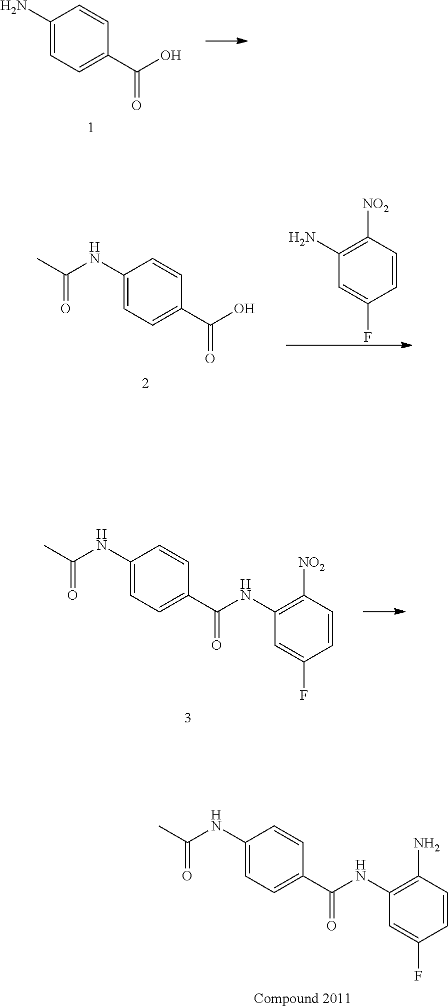 [SMILES: Nc1ccc(C(=O)O)cc1 ; CC(=O)Nc1ccc(C(=O)O)cc1 ; Nc1cc(F)ccc1[N+](=O)[O-] ; CC(=O)Nc1ccc(C(=O)Nc2cc(F)ccc2[N+](=O)[O-])cc1 ; CC(=O)Nc1ccc(C(=O)Nc2cc(F)ccc2N)cc1]

### Experimental Procedure:

Step 1: Ac₂O (5.7 ml, 60.3 mmol) was added to the solution of 4-amino-benzoic acid (6.86 g, 50.0 mmol) in pyridine (25 ml). The reaction was stirred at room temperature for 5 h. The solvent was removed in vacuo, and the residue was dispersed in water (100 ml) and acidified to pH 2-3 with concentrated hydrochloric acid. The resulting precipitate was collected by filtration, washed with water (30 ml) and dried to give 4-acetamido-benzoic acid as a pale yellow powder (7.80 g, 99%).

Step 2: To a solution of compound 2 (150 mg, 0.84 mmol) and 5-fluoro-2-nitro-aniline (1.0 eq) in pyridine (5 ml) was added POCl₃ (2.0 eq). The mixture was stirred for 1 h in an ice bath. After completion of the reaction, the mixture was quenched and extracted with EA to afford the crude product, compound 3, (100 mg, crude).

Step 3: To a solution of compound 3 (100 mg, crude) in MeOH (5 ml) was added Zn dust at 0° C., followed by NH₄Cl (3.0 eq). Then the reaction solution was stirred at r.t for 50 min. The mixture was concentrated to get a residue, which was purified by preparative-HPLC to afford compound 2011 (24 mg, 10% 2 steps). ¹H NMR (500 MHz, DMSO) δ 10.20 (s, 1H), 9.54 (s, 1H), 7.92 (d, J=8.5 Hz, 2H), 7.70 (d, J=9.0 Hz, 2H), 6.78 (d, J=6.0 Hz, 1H), 4.82 (s, 2H), 3.32 (s, 1H), 2.08 (s, 3H), 4H). LCMS: m/z=288 (M+H)⁺.

### Example 17

### HDAC Selectivity

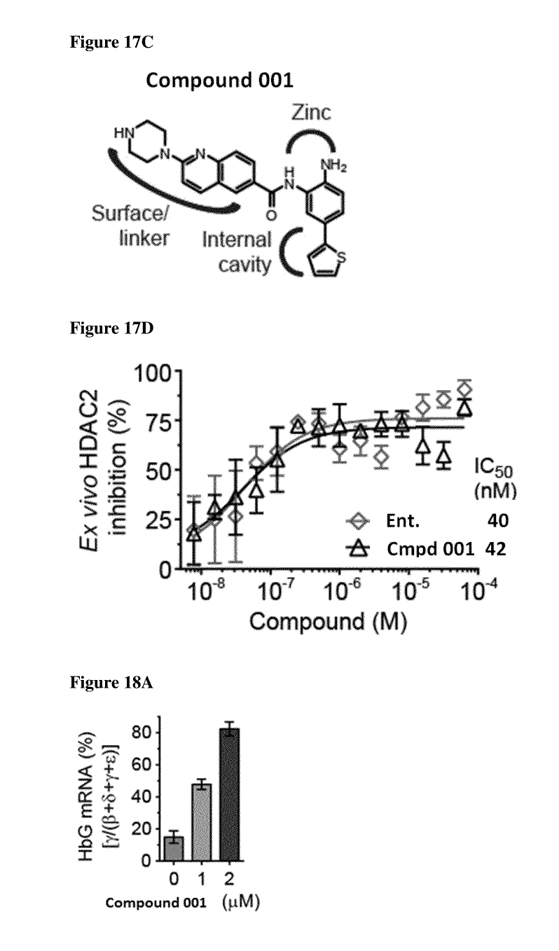

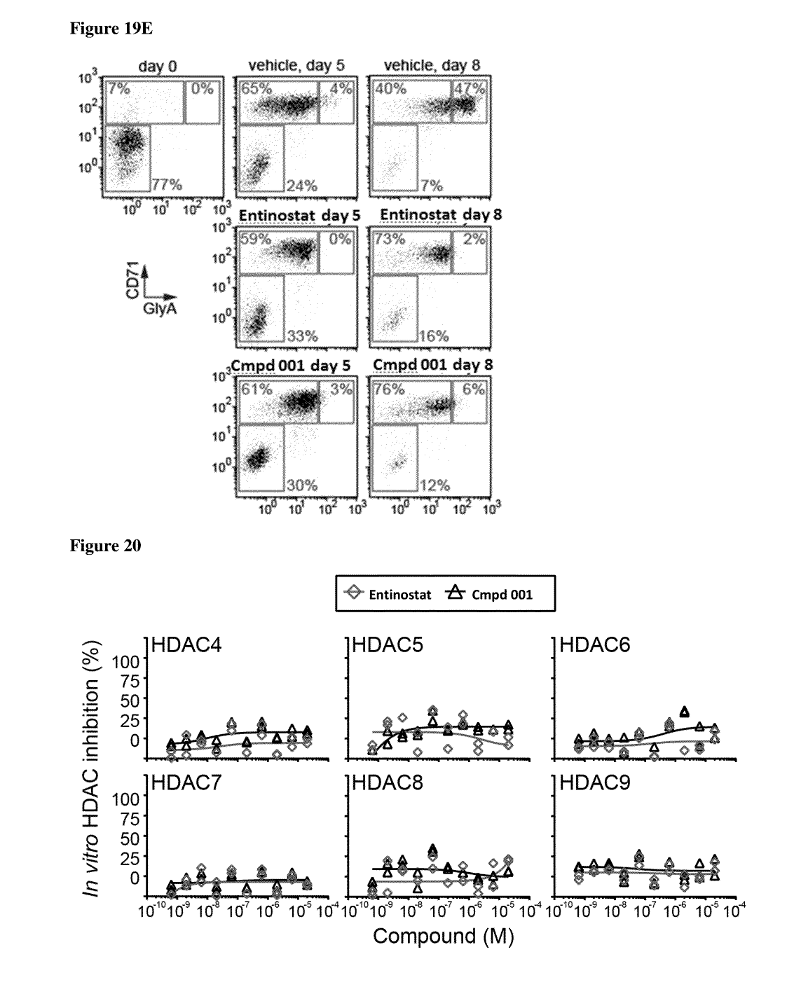

To confirm the HDAC inhibition profile of Entinostat and Compound 001, compounds were tested against each individual HDAC in an in vitro biochemical assay as previously described. Aminobenzamides have slow association rate constants, therefore a prolonged pre-incubation time of 24 hours is required to reach equilibrium. Entinostat had half maximal inhibitory concentration (IC₅₀) values of 37 nM, 47 nM, and 95 nM against HDAC1/2/3, respectively, values consistent with previous work (FIG. 17B). In contrast, Compound 001 had IC₅₀ values of 7 nM, 18 nM, and 1300 nM against HDAC1/2/3, respectively (FIG. 9A). As expected, no inhibition of HDACs 4-9 by Entinostat or Compound 001 was observed at concentrations as high as 20 μM (FIG. 20). Therefore, Entinostat is an HDAC1/2/3 inhibitor while Compound 001 is an HDAC1/2 inhibitor with an approximately 100-fold selectivity over HDAC3.

Next, the ability of Entinostat and Compound 001 to inhibit HDAC activity in primary hematopoietic progenitors was investigated. Using a live-cell-permeant acetylated substrate selective for HDAC2, we found Entinostat and Compound 001 had IC₅₀ values of 40 nM and 42 nM, respectively, demonstrating that these compounds are equally effective in crossing cellular membranes and reaching the HDAC target (FIG. 17D). To further validate HDAC inhibition by Compound 001 in cells, histone acetylation levels were examined by western blot (FIG. 9B). Compound 001 led to a dose-dependent accumulation of acetylation on histone H3 lysine 9 and 14 (H3K9/14ac), lysine 56 (H3K56), lysine 79 (H3K79ac), and H2B lysine 5 (H2BK5ac).

### Example 18

### Gata2 is Induced by Compound 001 in Culture of CD34+Cells Derived from Human Bone Marrow

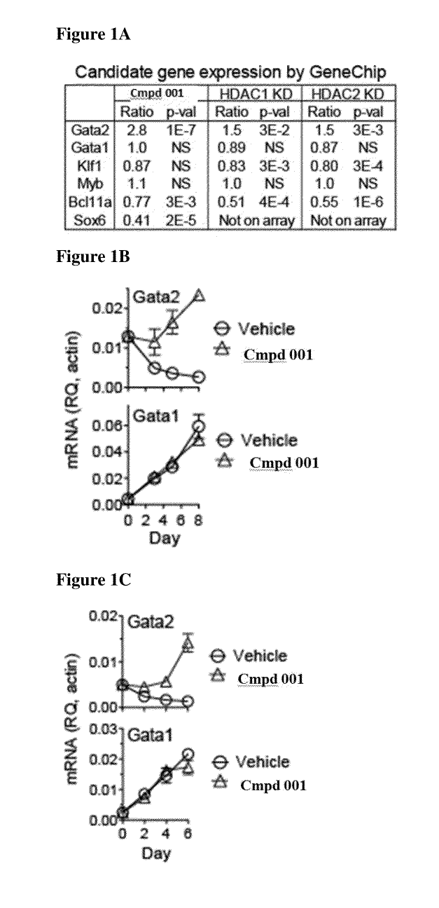

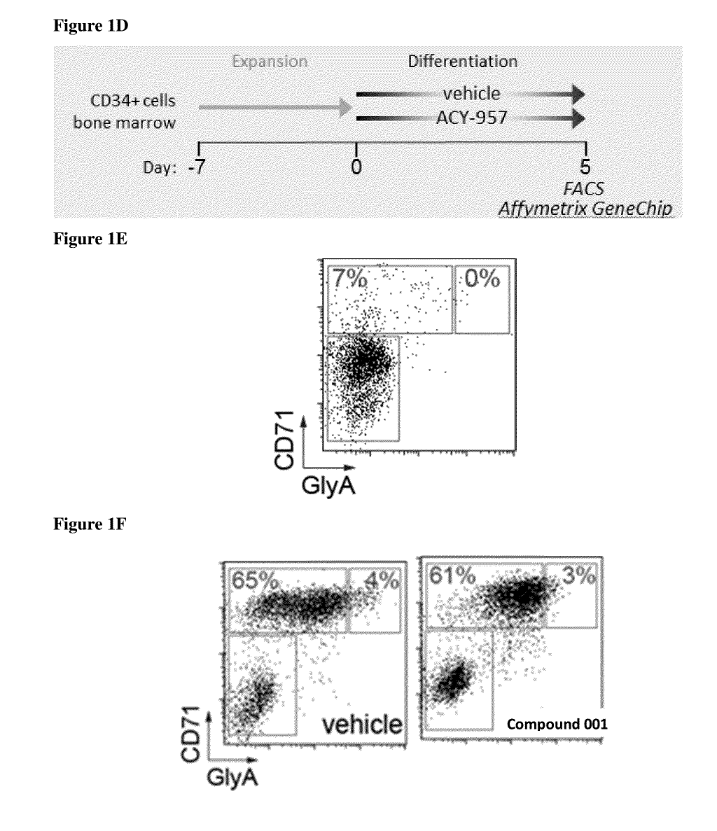

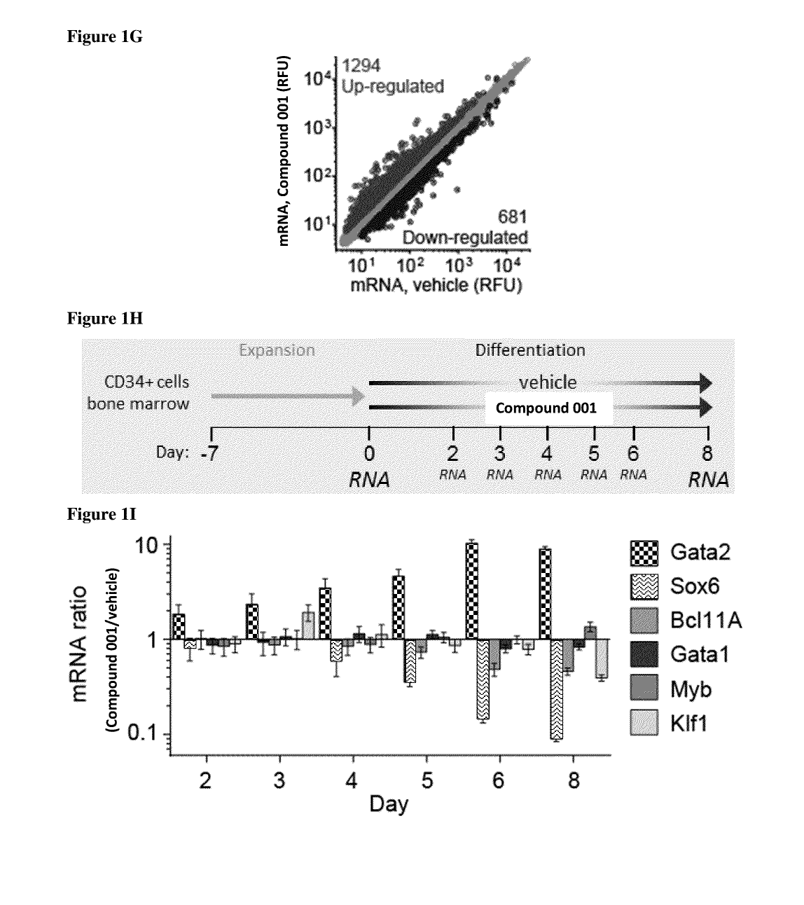

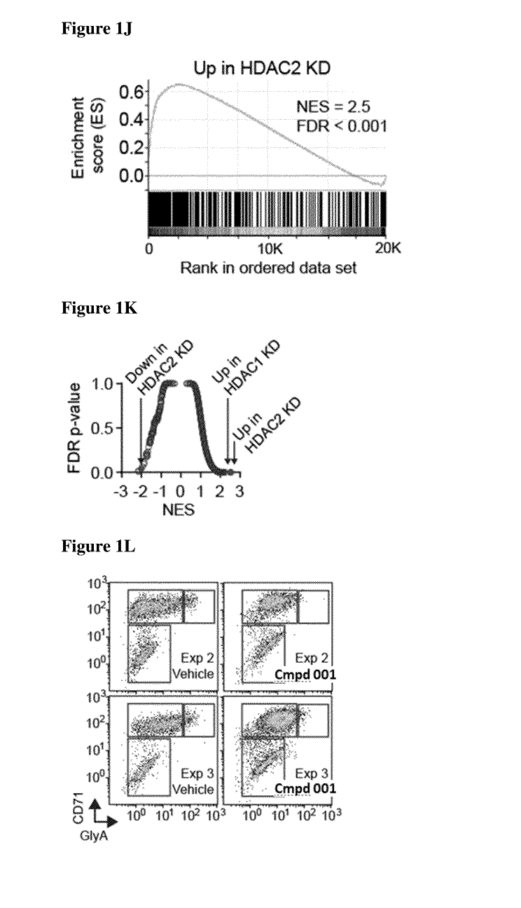

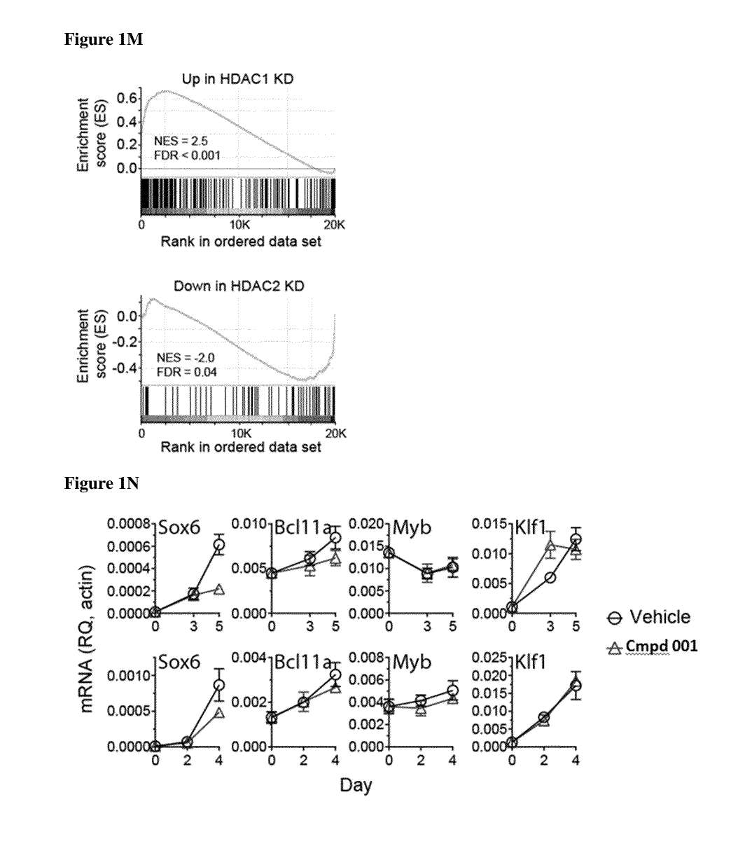

FIG. 1A shows Affymetrix GeneChip data of mRNA expression changes resulting from Compound 001 treatment (1 μM) or HDAC1 or HDAC2 short hairpin RNA knockdown, relative to untreated controls. The knockdown results were derived from an independent analysis of publically available raw data (Bradner et al., “Chemical genetic strategy identifies histone deacetylase 1 (HDAC1) and HDAC2 as therapeutic targets in sickle cell disease”, PNAS, vol. 107(28), pp. 12617-22 (2010)) and detection of mRNA by Affymetrix GeneChip. NS=not significant, FC=fold change resulting from Compound 001 treatment or knockdown.

FIG. 1B is a series of graphs that show quantitative real time PCR (QRT-PCR) data of mRNA expression changes over time resulting from Compound 001 treatment (1 μM for 8 days) in culture conditions supporting early erythroblasts. Gata2 mRNA was induced while Gata1 mRNA was unaffected. The culture system was as described by Sankaran et al., “Human fetal hemoglobin expression is regulated by the developmental stage-specific repressor BCL11A”, Science, vol. 322(5909), pp. 1839-42 (2008).

FIG. 1C is a series of graphs that show QRT-PCR data of mRNA expression changes over time resulting from Compound 001 treatment (1 μM for 6 days) in culture conditions supporting late erythroblasts. Gata2 mRNA was induced while Gata1 mRNA was unaffected. The culture system as described by Bradner et al., PNAS, vol. 107(28), pp. 12617-22 (2010).

### Example 19

### Treatment of Erythroid Progenitors with Various HDAC1,2 Inhibitors Leads to Induction of Gata2 mRNA

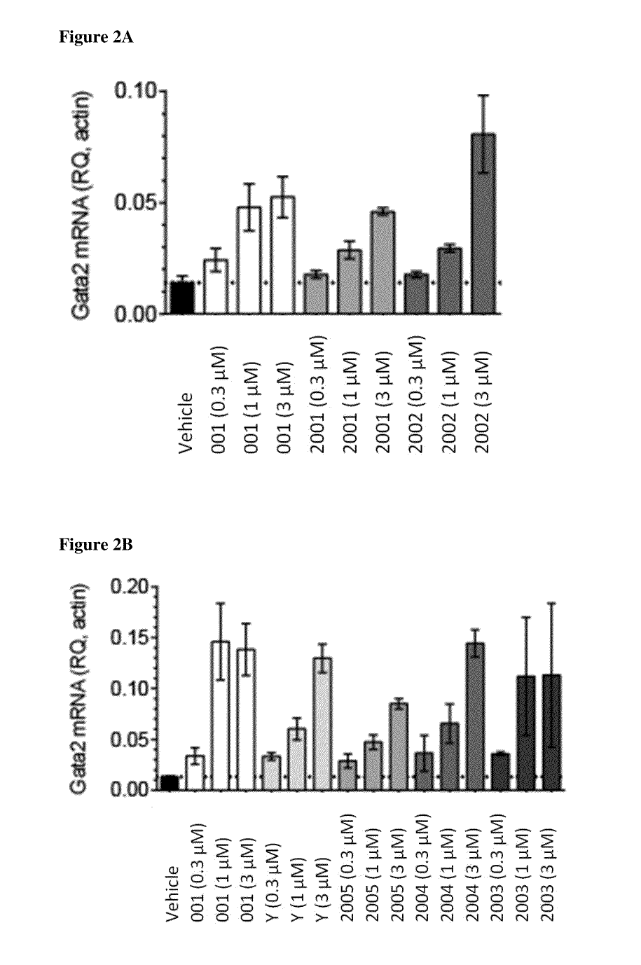

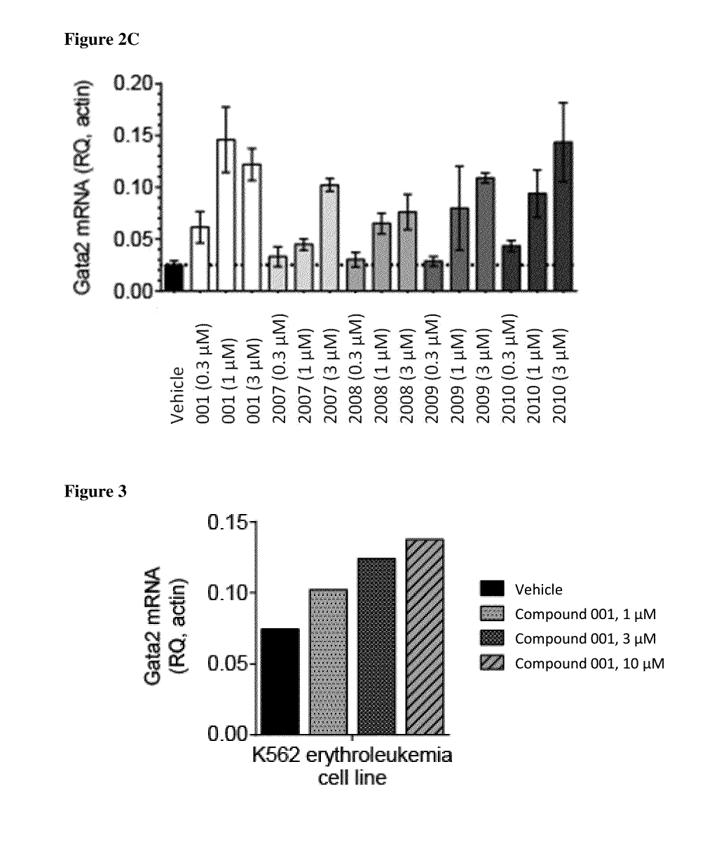

Human bone marrow derived CD34+ cells were expanded for 7 days as described by Sankaran et al., Science, vol. 322(5909), pp. 1839-42 (2008). Cells were then differentiated, in the presence of the indicated compound (i.e., Compounds 001, Compound Y, Compound 2005, Compound 2004, Compound 2003), for 3 days in media supporting erythropoiesis (Hu et al., “Isolation and functional characterization of human erythroblasts at distinct stages: implications for understanding of normal and disordered erythropoiesis in vivo”, Blood, vol. 121(16), pp. 3246-53 (2005)). FIG. 2A, FIG. 2B, and FIG. 2C each represents an experimental series performed on different days and with different donor cells.

### Example 20

### Induction of Gata2 by HDAC1,2 Inhibitors is Dose Dependent

K562 erythroleukemia cells were treated with Compound 001 for 3 days. A dose dependent response in Gata2 mRNA was observed, as shown in FIG. 3.

### Example 21

### Beta-Thalassemia Patient Samples Treated with Selective HDAC1,2 Inhibitors have Elevated Levels of Gata2 mRNA

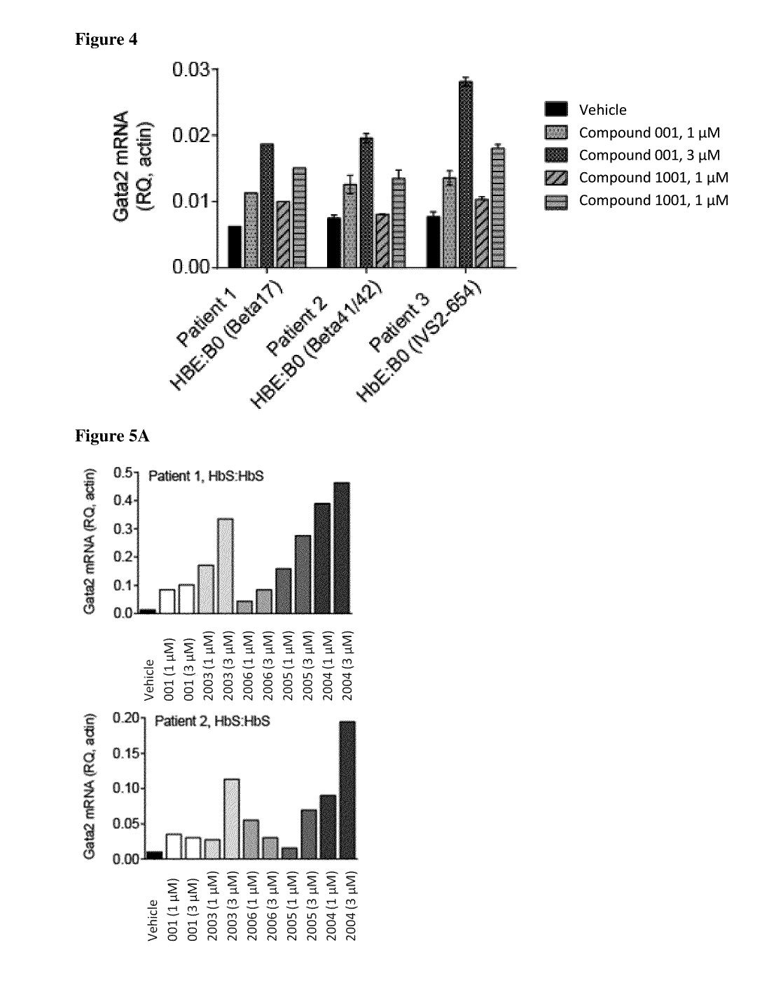

Peripheral blood-derived mononuclear cells from hemoglobin E:Beta zero (HbE:B0) compound heterozygous patient samples were expanded for 7 days as described by Sankaran et al., Science, vol. 322(5909), pp. 1839-42 (2008). The cells were then differentiated, in the presence of the indicated compound (i.e., Compound 001, Compound 1001), for 3 days in media supporting erythropoiesis (Hu et al., Blood, vol. 121(16), pp. 3246-53 (2005)). Results are shown in FIG. 4.

### Example 22

### Sickle Cell Patient Samples Treated with Selective HDAC1,2 Inhibitors have Elevated Levels of Gata2 mRNA

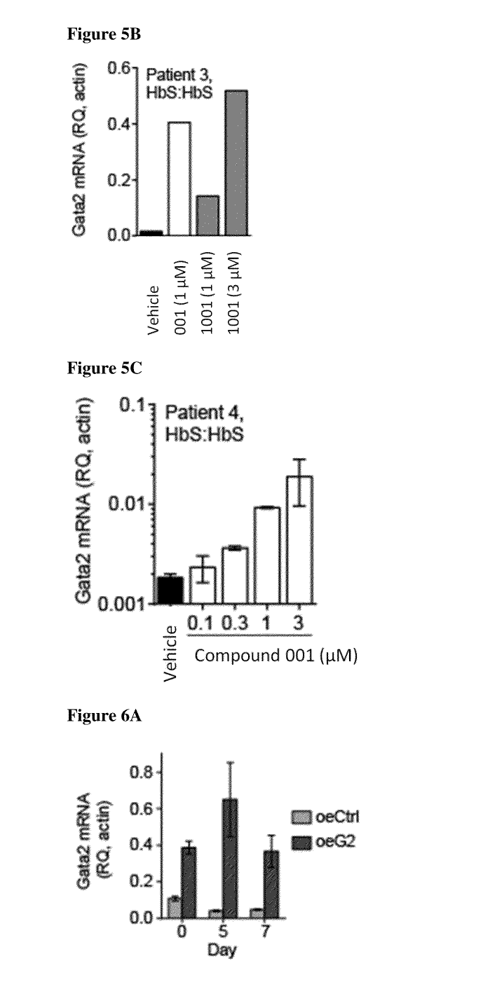

Peripheral blood-derived mononuclear cells from HbS homozygote patients were expanded for 7 days as described by Sankaran et al., Science, vol. 322(5909), pp. 1839-42 (2008). Cells were then differentiated, in the presence of the indicated compound (i.e., Compounds 001, Compound 2003, Compound 2006, Compound 2005, Compound 2004), for 3 days in media supporting erythropoiesis (Hu et al., Blood, vol. 121(16), pp. 3246-53 (2005)). FIG. 5A, FIG. 5B, and FIG. 5C each represents an experimental series performed on different days and with different donor cells.

### Example 23

### Pharmacological Inhibition of Histone Deacetylases 1 and 2 (HDAC1/2) Induces Fetal Hemoglobin (HbF) Through Activation of Gata2

Induction of HbF is an established therapeutic strategy for the treatment of sickle cell disease, and could also be effective in treating beta-thalassemia. Genetic ablation of HDAC1 or HDAC2, but not HDAC3, results in the induction of the fetal beta-like globin gene (HbG) transcript (Bradner et al., PNAS, 2010; 107(28):12617-22). It has been previously shown that selective chemical inhibitors of HDAC1/2 elicit a dose and time dependent induction of HbG mRNA and HbF protein in cultured human CD34+ bone marrow cells undergoing erythroid differentiation (Shearstone J S, ASH Annual Meeting Abstracts, 2012). This work utilized Compound 001, a selective inhibitor of HDAC1/2, to discover a novel role for Gata2 in the activation of HbG.

To identify genes affected by HDAC1/2 inhibition, CD34+ bone marrow cells undergoing erythroid differentiation were treated with Compound 001 or vehicle, followed by mRNA expression profiling. See FIGS. 1A, 1B, and 1C. Among the genes differentially regulated by both pharmacological inhibition and genetic ablation of HDAC1/2 were Bcl11a and Sox6, known HbG repressors, and Gata2, a potential HbG activator. Quantitative real time PCR (QRT-PCR) time course experiments confirmed that Compound 001 treatment leads to a 2-fold and 10-fold decrease in Bcl11a and Sox6, respectively, and an 8-fold increase in Gata2 mRNA (FIGS. 1B, 1C, 1H, and 1N). Unlike Bcl11a and Sox6, Gata2 induction by Compound 001 was highly correlated with HbG induction, suggesting a possible role for this transcription factor in the direct activation of HbG.

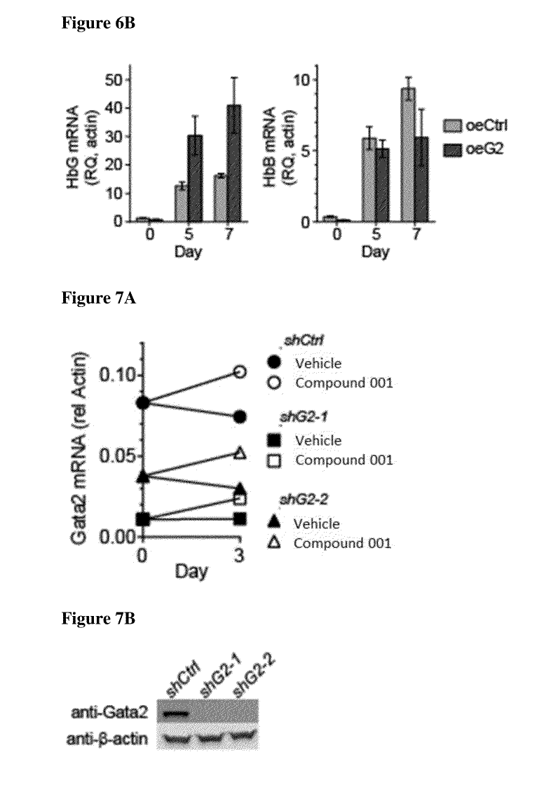

To investigate this possibility, lentiviral infection was utilized to overexpress full length Gata2 transcript in differentiating primary erythroblasts. FIGS. 6A-B show that overexpression of Gata2 induces gamma globin in erythroid progenitors derived from CD34+ human bone marrow cells. In FIG. 6A, expanded hematopoietic progenitors were infected with lentivirus carrying the full length Gata2 gene (oeG2) or green fluorescent protein control (oeCtrl). Transduced cells were selected by puromycin treatment and then shifted to culture conditions supporting differentiation of cells into early erythroblasts (Day 0). RNA was isolated at indicated time points, and the level of Gata2 mRNA was determined by quantitative real time PCR (QRT-PCR). FIG. 6B shows the HbG (left graph) and HbB (right graph) mRNA levels for the cells in FIG. 6A. After 5 days of differentiation, Gata2 overexpression resulted in a 2.5-fold increase in HbG mRNA, while the level of the major adult beta-like globin chain (HbB) mRNA was unaffected. HbG mRNA remained elevated by Gata2 overexpression at day 7 of differentiation, while HbB was reduced by 1.6-fold. Gata2 overexpression appeared to have minimal effect on cell differentiation, as determined by the cell surface markers CD71 and GlycophorinA, a finding consistent with observations in Compound 001 treated cells with elevated Gata2.

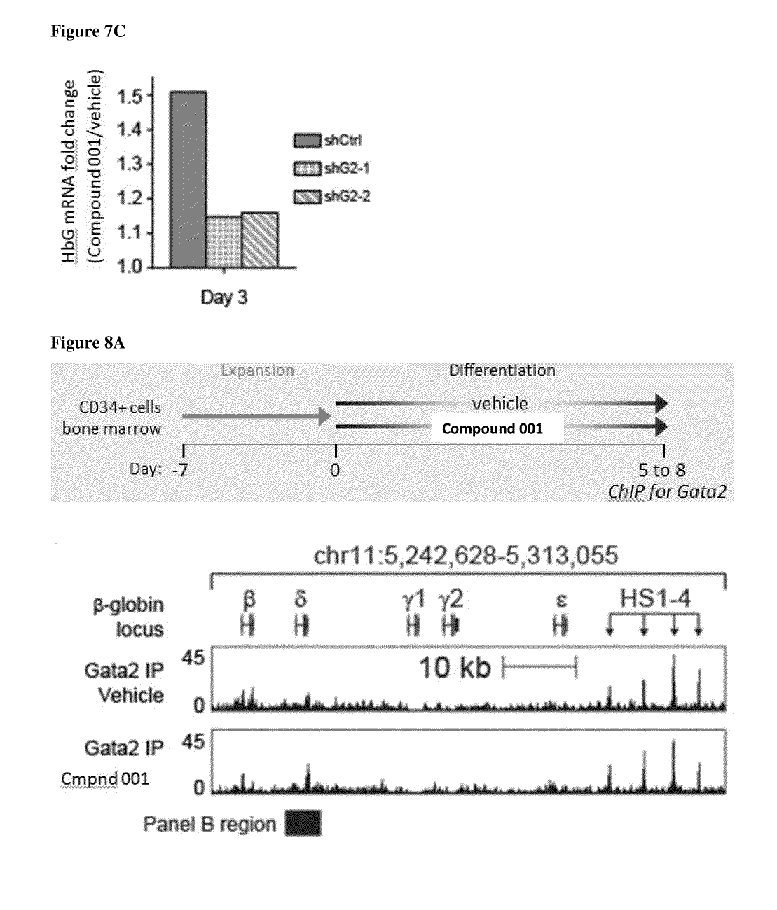

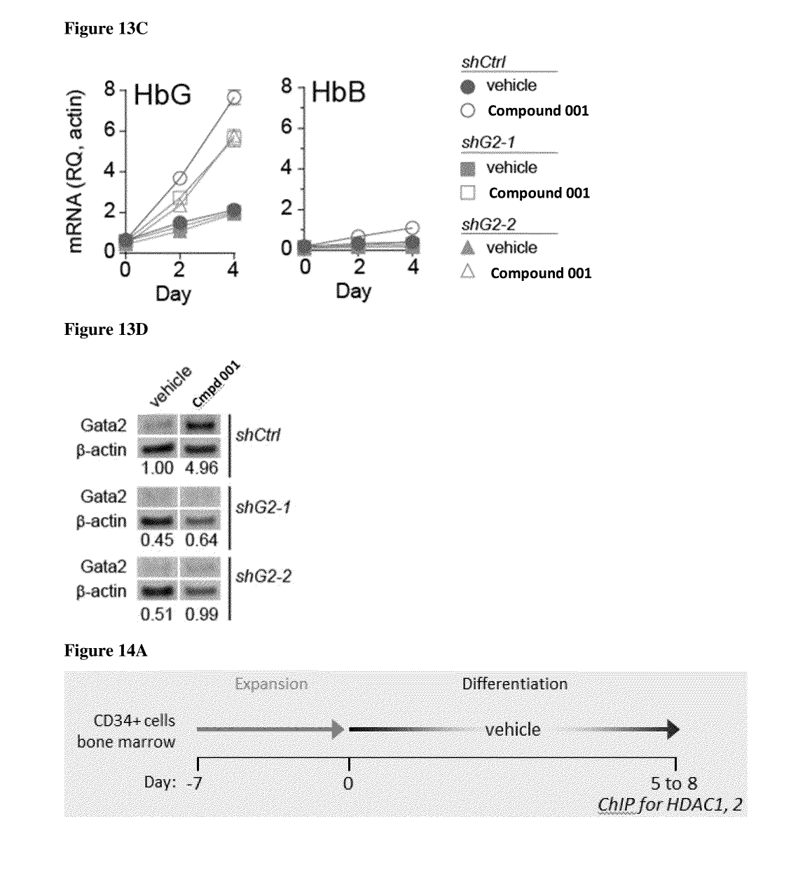

Furthermore, lentiviral delivery of short hairpin RNA (shRNA) targeting Gata2, attenuated HbG induction by Compound 001. FIGS. 13A-D and 7A-C show that knockdown of Gata2 attenuates HbG induction by Compound 001 in erythroid progenitors derived from CD34+ human bone marrow cells and the erythroleukemia cell line K562. In FIG. 13A-D, expanded hematopoietic progenitors were infected with lentivirus carrying short hairpin RNAs (shRNA) directed against the Gata2 gene (shG2-1, shG2-2) or a non-targeting control shRNA (shCtrl). Transduced cells were selected by puromycin treatment. Puromycin was removed (Day 0) and then cells were cultured for an additional four days in the presence of 0.5 micromolar Compound 001, 1 micromolar Compound 001, or vehicle control. RNA was isolated at the indicated time points and the level of Gata2 mRNA was determined by quantitative real time PCR (QRT-PCR). In FIG. 7A, K562 cells were infected with lentivirus as described above. Puromycin was removed (Day 0) and then cells were cultured for an additional three days in the presence of 1 micromolar Compound 001, or vehicle control. RNA was isolated at indicated time point and the level of Gata2 mRNA was determined by quantitative real time PCR (QRT-PCR). In FIG. 7B, protein levels at Day 3 were determined by Western blot using antibodies against Gata2 and beta-actin as a loading control. FIG. 7C shows HbG mRNA levels in Compound 001 treated cells. Data was first normalized to beta-actin control and then expressed relative to vehicle treated cells.

These data suggest that elevated levels of Gata2 resulting from HDAC1/2 inhibition is sufficient to induce HbG at early stages of erythroid cell differentiation.

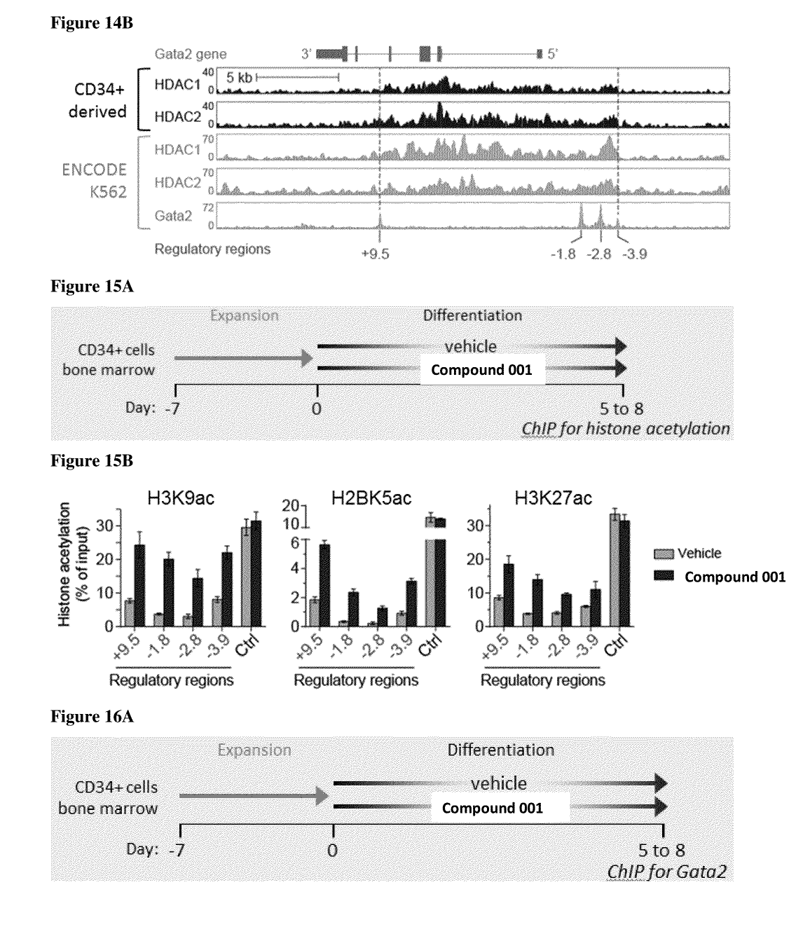

To understand how HDAC1/2 inhibition drives Gata2 activation, chromatin immunoprecipitation coupled with either next generation sequencing (ChIP-seq) or QRT-PCR was performed in Compound 001 and vehicle treated cells. FIG. 15B shows that Compound 001 treatment results in elevated histone acetylation and Gata2 binding at known Gata2 regulatory regions. In FIG. 15B, chromatin was immunoprecipitated using antibodies that bind histone H3 lysine 9 acetylation (H3K9ac), histone H2B lysine 5 (H2BK5ac), or histone H3 lysine 27 (H3K27ac) marks and then detected using QRT-PCR. Treatment of differentiating primary erythroid progenitors with 1 micromolar of Compound 001 increased H3K9ac, H2BK5ac, and H3K27ac within regions known to regulate Gata2 expression (+9.5, −1.8, −2.8, and −3.9 regions as described by Martowicz et al. 2005).

HDAC1 and HDAC2 were present throughout the Gata2 gene body and promoter regions, and HDAC1/2 binding levels were highly correlated, suggesting co-occupancy of these enzymes at this locus. Compound 001 treatment led to elevated histone acetylation at previously described Gata2 gene regulatory regions (Bresnick E H, Lee H Y, Fujiwara T, Johnson K D, Keles S. GATA switches as developmental drivers. The Journal of biological chemistry. 2010; 285(41):31087-31093.). Specifically, the −1.8 kb and −2.8 kb regulatory regions showed a 6-fold increase in histone H3K9, H2BK5, and H3K27 acetylation, while the +9.5 kb and −3.9 kb regions showed a 3-fold increase. The Gata2 protein showed increased binding at these regulatory regions in response to Compound 001 treatment, with a maximum increase of 3-fold at the −1.8 kb region. This finding is consistent with the known positive autoregulation of the Gata2 gene. Taken together, these data suggest that selective inhibition of HDAC1/2 leads to elevated Gata2 through acetylation-induced activation of a positive autoregulatory loop.

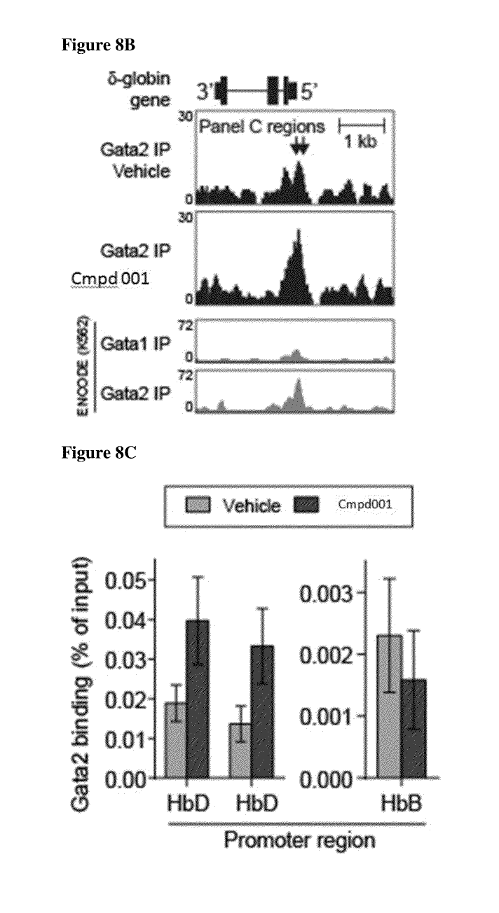

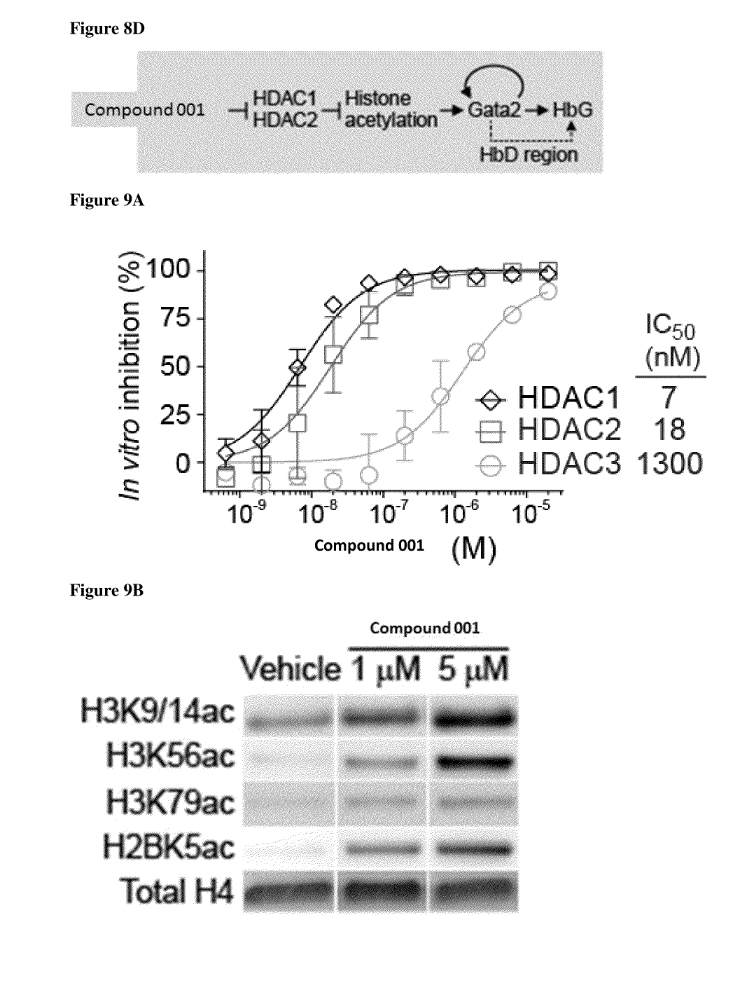

The tight temporal correlation between Gata2 and HbG activation following HDAC1/2 inhibition argues that Gata2 may affect the beta-globin locus directly. ChIP-seq data across the 70-kb beta-globin locus demonstrated that Compound 001 treatment altered Gata2 binding only at a single region, lying within the promoter for delta globin. FIGS. 8A-D show Compound 001 treatment results in elevated Gata2 binding near the delta globin promoter. FIG. 8A shows Gata2 binding at the beta-like globin gene cluster using ChIP-seq in differentiating primary erythroid progenitors treated with 1 micromolar of Compound 001 or vehicle control. Compound 001 treatment resulted in elevated Gata2 binding at a single region within the beta-like globin gene cluster, located at the delta globin promoter. FIG. 8B shows an expanded view of the data presented in FIG. 8A at the delta globin gene locus. In FIG. 8C, the ChIP-seq results in FIG. 8A were validated in a second experimental series using QRT-PCR and two primer sets directed to the delta globin promoter. A primer set at the beta globin promoter was used as a control. FIG. 8D shows a proposed mechanism by which HDAC1,2 selective inhibitor induces gamma globin. This region is suspected in playing a role in switching from fetal to adult globin during development, as naturally occurring deletions of this region are associated with elevated fetal hemoglobin in adults (Sankaran V G, Xu J, Byron R, et al. A functional element necessary for fetal hemoglobin silencing. The New England journal of medicine. 2011; 365(9):807-814.). Whether the change in GATA2 binding to this region is responsible for the increased expression of HbG in cells treated with HDAC1/2-selective inhibitors is under investigation.

### Example 24

### Pharmacological Inhibition of Histone Deacetylases 1 and 2 Induces Fetal Hemoglobin Through Activation of Gata2

Class I histone deacetylases (HDAC) are zinc-dependent, nuclear enzymes that remove acetyl groups, primarily from histones. Examples of Class I HDACs are HDAC1, 2, 3 and 8. HDACs oppose the function of histone acetyltransferases (HAT), and affect chromatin structure and gene expression. It is known that non-selective HDAC inhibitors, such as vorinostat (SAHA), panobinostat (LBH-589), romidepsin (FK228) and givinostat, induce fetal globin. See, for example: Atweh et al. “Sustained induction of fetal hemoglobin by pulse butyrate therapy in sickle cell disease” Blood, 1999, 93(6):1790-7; Ronzoni et al. “Modulation of gamma globin genes expression by histone deacetylase inhibitors: an in vitro study” British Journal of Haematology, 2014, 165(5):714-721; Bradner et al. “Chemical genetic strategy identifies histone deacetylase 1 (HDAC1) and HDAC2 as therapeutic targets in sickle cell disease” Proceedings of the National Academy of Sciences of the United States of America, 2010, 107(28):12617-12622; and Cao and Stamatoyannopoulos “Histone deacetylase inhibitor FK228 is a potent inducer of human fetal hemoglobin” American Journal of Hematology, 2006, 81(12):981-983.

The rationale for selective HDAC1,2 inhibition is due to the fact that knockdown of HDAC1 or HDAC2, but not HDAC3, by shRNA leads to gamma globin activation. See, for example, Bradner et al., PNAS 2010; Xu et al., PNAS 2013; and Witter et al., Bioorg & Med Chem Lett. 2008. In addition, benzamide derivative are 10 to 100 fold selective for HDAC1 and HDAC2 over HDAC3.

Selective inhibition of HDAC1/2 by Compound 001 induced histone acetylation. In FIG. 9A, various concentrations of Compound 001 were tested to determine the in vitro inhibition of either HDAC1, HDAC2, or HDAC3. The results show that Compound 001 was much more selective for HDAC1 (IC₅₀ of 7 nM) and HDAC2 (IC₅₀ of 18 nM) than HDAC3 (IC₅₀ of 1300 nM). In FIG. 20, various concentrations of Compound 001 were tested to determine the in vitro inhibition of either HDAC4, HDAC5, HDAC6, HDAC7, HDAC8, or HDAC9. The results show that Compound 001 was much more selective for HDAC1 and HDAC2 than any of HDAC4, HDAC5, HDAC6, HDAC7, HDAC8, or HDAC9. In FIG. 9B, Compound 001 was tested at concentrations of 1 and 5 μM to determine the extent of induction of acetylation at various sites. The results show that Compound 001, which selectively inhibits HDAC1/2, induced histone acetylation.

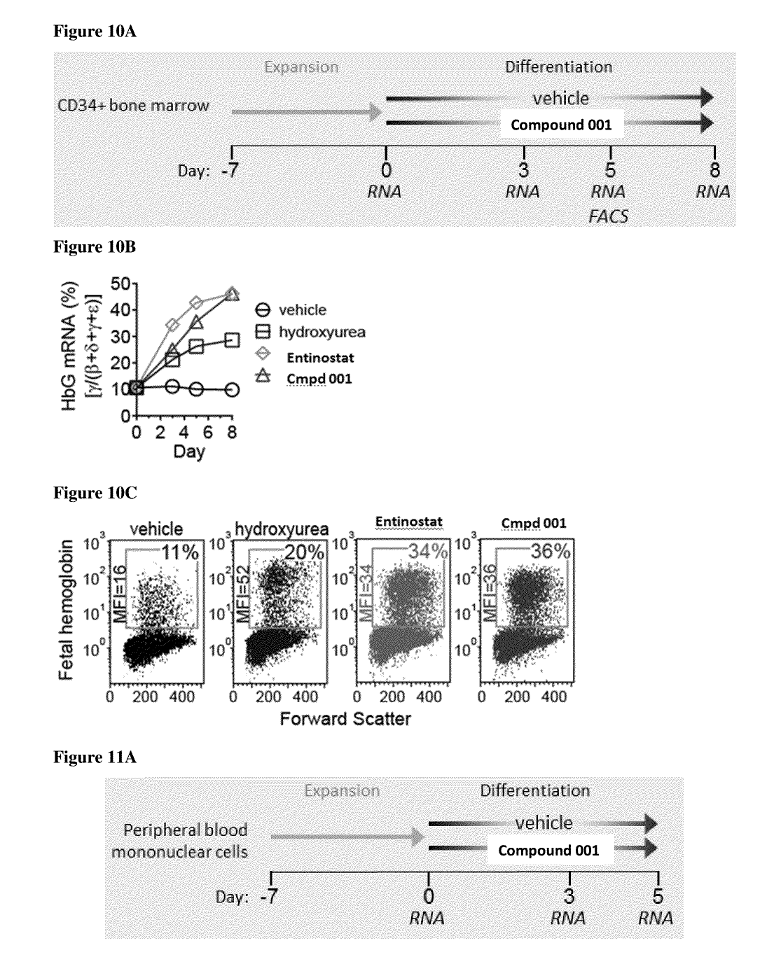

Compound 001 induced HbG and HbF (fetal hemoglobin). Briefly, as shown in FIG. 10A, CD34+ bone marrow cells were cultured and expanded for 7 days. Then, the cells were differentiated by exposure to either vehicle (control) or Compound 001. RNA samples of HbG were taken at days 0, 3, 5, and 8. FIG. 10B is a graph that shows that Compound 001 induced HbG. Staining cells with a FITC-conjugated anti-HbF antibody and flow cytometry was used to measure induction of fetal hemoglobin. FIG. 10C is a series of scatter plots that show that Compound 001 induced HbF.

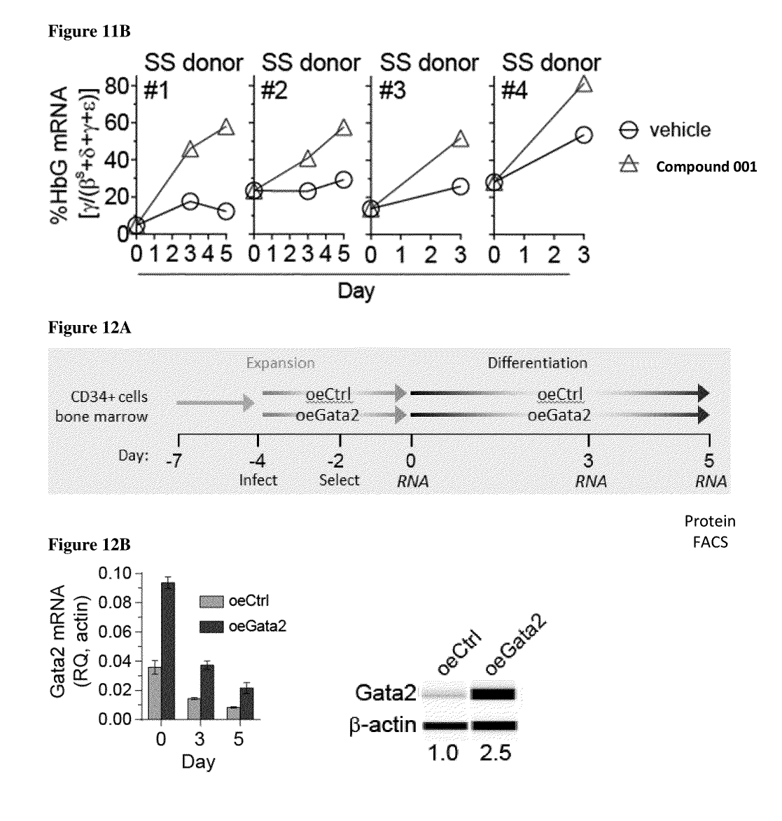

Compound 001 induced HbG in sickle donor cells. Briefly, as shown in FIG. 11A, peripheral blood mononuclear cells from four different donors were cultured and expanded for 7 days. Then, the cells from each donor were differentiated by exposure to either vehicle (control) or Compound 001. RNA samples of HbG were taken from each group of donor cells at days 0, 3, and 5. The results are shown in FIG. 11B, which shows that Compound 001 induced HbG in sickle donor cells.

Compound 001 or HDAC1/2 KD induced Gata2. Briefly, as shown in FIG. 1D, CD34+ bone marrow cells were cultured and expanded for 7 days. Then, the cells were differentiated by exposure to either vehicle (control) or Compound 001. FACS was performed at day 5. FIG. 1E shows a scatter plot of CD71 v. GlyA. FIG. 1F shows scatter plots of CD71 v. GlyA for vehicle (FIG. 1F, left panel) and Compound 001 (FIG. 1F, right panel). FIG. 1G shows a scatter plot of Compound 001 mRNA v. vehicle mRNA. The Table below shows candidate gene expression ratios for Compound 001, HDAC1 KD, and HDAC2 KD.

| | Gata2 | Gata1 | KLF1 | Myb | Bcl11a | Sox6 |
|---|---|---|---|---|---|---|
| Cmpd. 001 | 2.8 | 1.0 | 0.9 | 1.1 | 0.8 | 0.4 |
| HDAC1 KD | 1.5 | 0.9 | 0.8 | 1.0 | 0.5 | Not on array |
| HDAC2 KD | 1.5 | 0.9 | 0.8 | 1.0 | 0.6 | Not on array |

Compound 001 induced Gata2. Briefly, as shown in FIG. 1H, CD34+ bone marrow cells were cultured and expanded for 7 days. The cells were then differentiated by exposure to either vehicle or Compound 001. RNA was measured at days 0, 2, 3, 4, 5, 6, and 8. FIG. 1B-C, top panels, is a graph that shows that Gata2 mRNA was induced by Compound 001. FIG. 1B-C, bottom panels, is a graph that shows that Gata1 mRNA was not induced by Compound 001. FIG. 1I is a graph that shows the mRNA ratio of Compound 001/vehicle for Gata2, Sox6, Bcl11A, Gata1, Myb, and Klf1 at days 2, 3, 4, 5, 6, and 8.

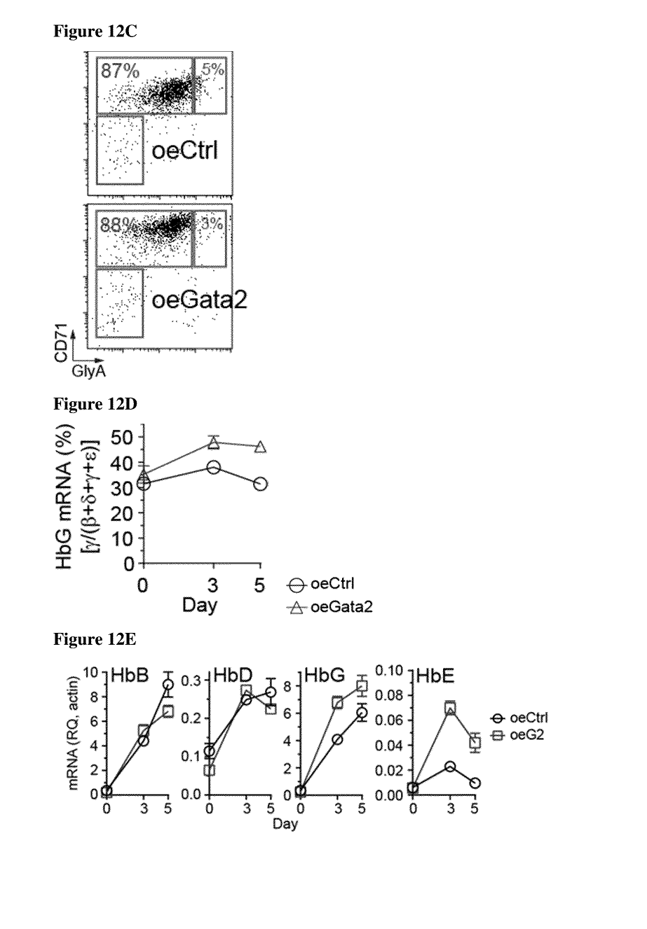

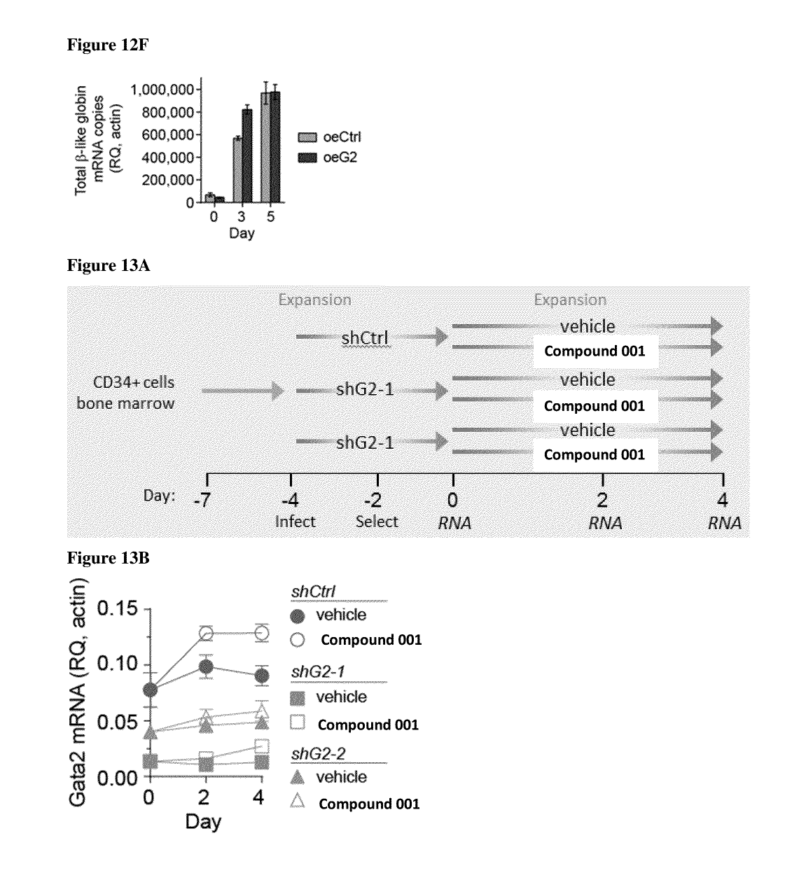

Gata2 overexpression induced HbG. Briefly, CD34+ bone marrow cells were subject to expansion and differentiation according to the protocol in FIG. 12A. FIG. 12B, left panel, is a graph that shows Gata2 expression for oeCtrl and oeGata2 on days 0, 3, and 5. FIG. 12B, right panel, is a photo of a gel showing Gata2 and β-actin for oeCtrl and oeGata2. FIG. 12C, top panel, is a scatter plot of CD71 v. GlyA for oeCtrl at day 5. FIG. 12C, bottom panel, is a scatter plot of CD71 v. GlyA for oeGata2 at day 5. FIG. 12D, is a graph that shows the percent HbG mRNA relative to total beta-like globin mRNA at days 0, 3, and 5 for oeCtrl and oeGata2. FIG. 12E is a graph that shows HbB, HbD, HbG, and HbE mRNA at days 0, 3, and 5 for oeCtrl and oeGata2.

Gata2 knockdown attenuated HbG induction by Compound 001. Briefly, CD34+ bone marrow cells were expanded according to the protocol in FIG. 13A. FIG. 13B is a graph that shows Gata2 mRNA for each of shCtrl, shG2-1, and shG2-2 that was treated with vehicle or Compound 001. FIG. 13C, left panel is a graph that shows HbG mRNA for each of shCtrl, shG2-1, and shG2-2 that was treated with vehicle or Compound 001. FIG. 13C, right panel is a graph that shows HbB mRNA for each of shCtrl, shG2-1, and shG2-2 that was treated with vehicle or Compound 001. FIG. 13D is a western blot that shows Gata2 protein for each of shCtrl, shG2-1, and shG2-2 cells treated with vehicle or Compound 001.

HDAC1/2 co-occupy the Gata2 locus. Briefly, CD34+ bone marrow cells were expanded and differentiated according to the protocol in FIG. 14A. FIG. 14B shows the Gata2 gene in relation to HDAC1 and HDAC2 protein binding in CD34+ derived cells and K562 cells.

Compound 001 hyperacetylated histones at Gata2 regulatory regions. Briefly, CD34+ bone marrow cells were expanded and differentiated according to the protocol in FIG. 15A. FIG. 15B is a series of graphs that show hisone acetylation at various Gata2 regulatory regions for vehicle or Compound 001 at positions H3K9 (left panel), H2BK5 (center panel), and H3K27 (right panel).

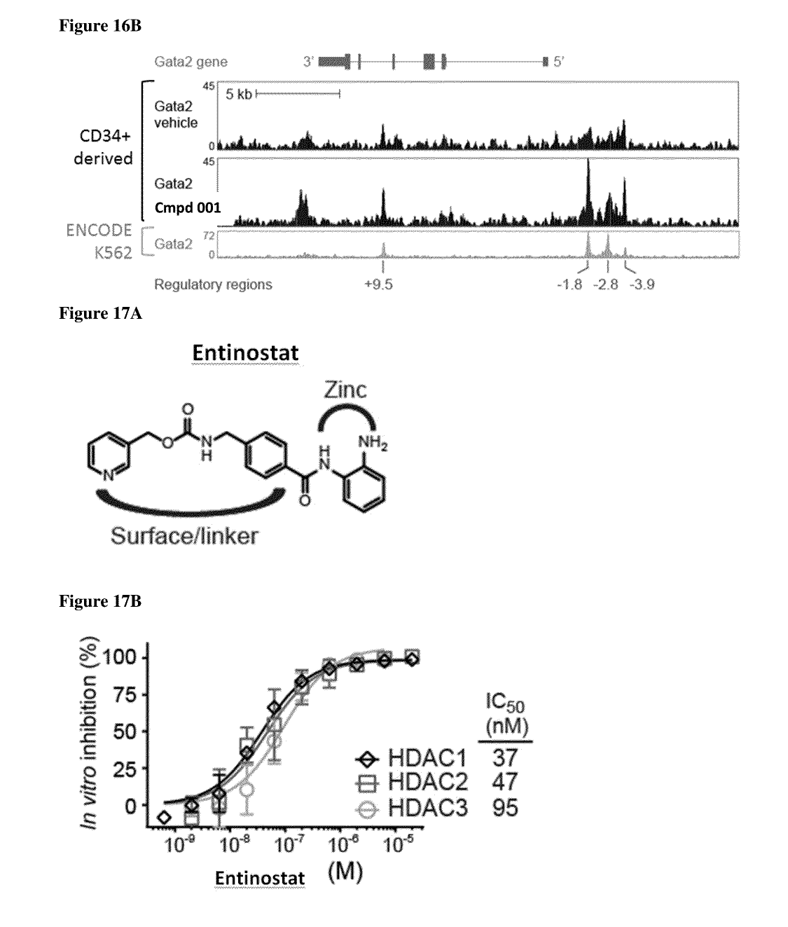

Compound 001 increased Gata2 binding at Gata2 regulatory regions. Briefly, CD34+ bone marrow cells were expanded and differentiated according to the protocol in FIG. 16A. FIG. 16B shows the Gata2 gene in relation to CD34+ cells treated with vehicle and Compound 001, and K562 cells.

Compound 001 increased Gata2 binding at the HbD promoter. Briefly, CD34+ bone marrow cells were expanded and differentiated according to the protocol in FIG. 8A. FIG. 8A also shows the Gata2 promoter in relation to CD34+ cells treated with vehicle and Compound 001, and K562 cells. FIG. 8C is a graph that shows Gata2 binding at the HbD promoter for two sets of cells treated with either vehicle or Compound 001.

Increased Gata2 binding at the HbD promoter may alter HbG expression. The region upstream of the HbD gene is required for fetal hemoglobin silencing. This region overlaps with increase in Gata2 occupancy upon treatment with Compound 001. See, for example, FIG. 2A of Sankaran V G et al., New England J. Med., 2011, 365(9):807-14.

In conclusion, HDAC1/2 inhibition (Compound 001) induced HbG in primary erythroid progenitors, in part, through activation of Gata2. Compound 001 induced HbG and Gata2 expression. Gata2 overexpression alone led to elevated HbG. Gata2 knockdown attenuated HbG induction by Compound 001. HDAC1 and 2 co-occupied the Gata2 locus. Compound 001 increased histone acetylation at Gata2 regulatory regions. Compound 001 increased Gata2 occupancy at Gata2 regulatory regions.

How is Gata2 regulating HbG expression? Direct regulation is suggested by the timing of events and ChIP-seq at the HbD promoter. See FIG. 8D.

### Example 25

### Selective Inhibition of HDAC1/2 by Compound 001 Induces HbG mRNA and HbF Protein

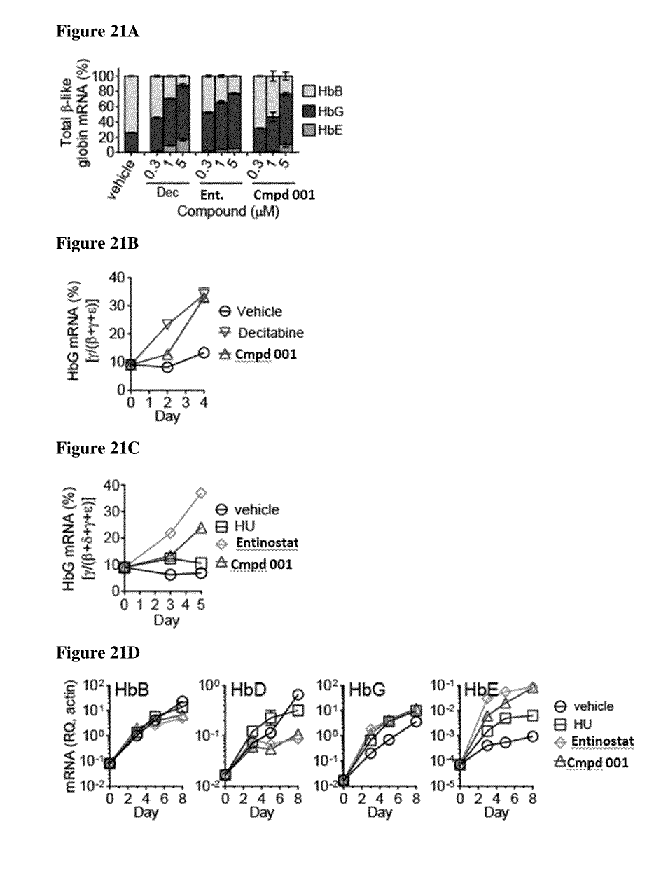

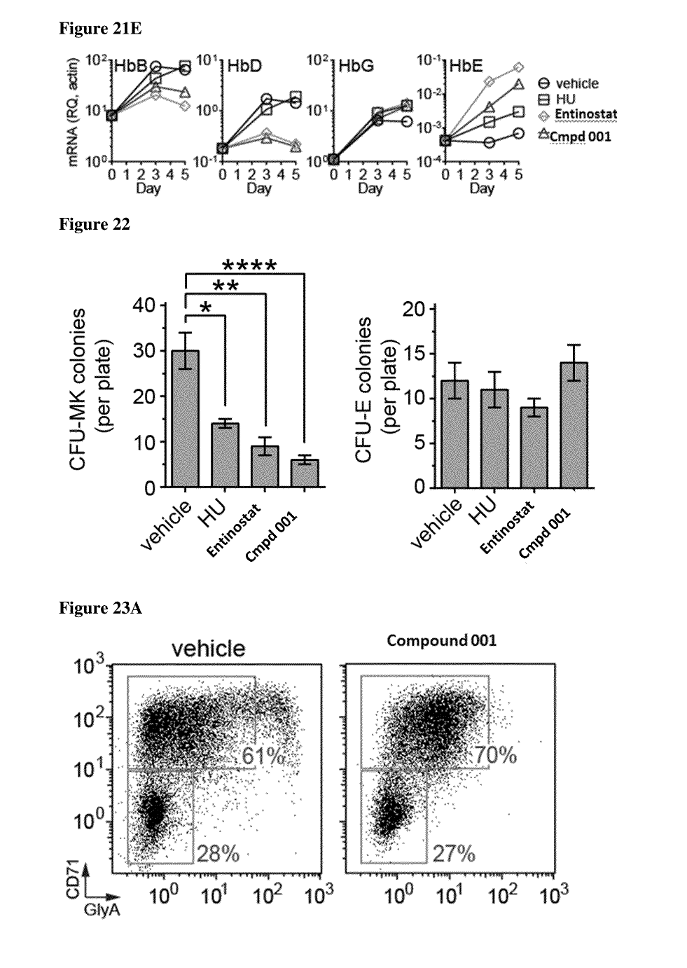

To evaluate the ability of Compound 001 to activate HbG, two distinct 2-phase culture systems were utilized, referred to as CS1 and CS2, to derive erythroid progenitors and erythroblasts from CD34+ human bone marrow cells. HDAC inhibition, either by 1 μM Compound 001 or Entinostat, led to a dose- and time-dependent induction in % HbG during the differentiation phase in both culture systems (FIG. 10B and FIGS. 21A-C). Both Entinostat and Compound 001 increased % HbG from 10% to 46%, a level of induction equivalent to, or greater than, that of the known HbG inducers decitabine and hydroxyurea at 1 μM and 30 μM, respectively.

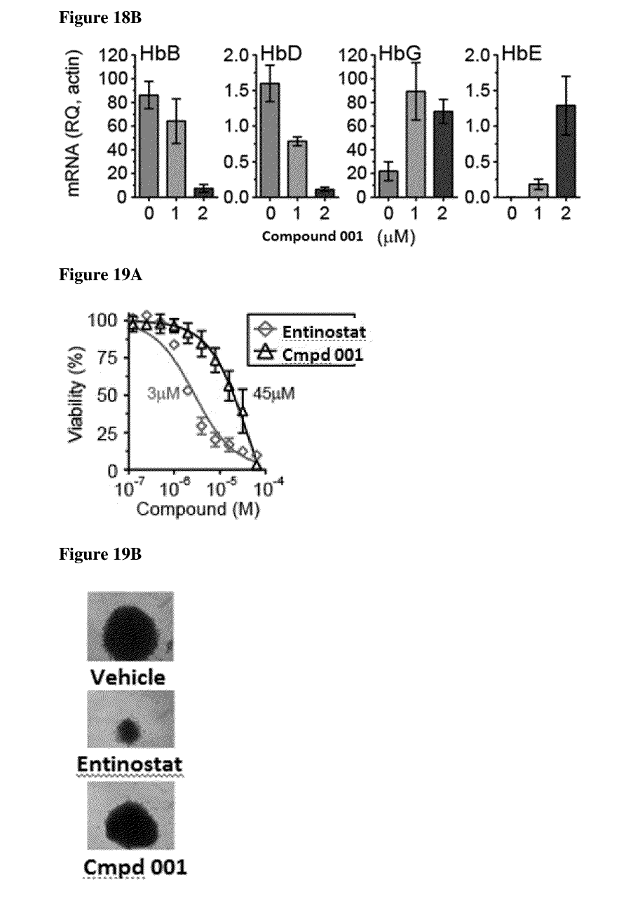

Compound 001 also increased % HbG in a dose-dependent manner in burst forming unit erythroid (BFU-E) colonies (FIG. 18A) and in cells derived from patients homozygous for the sickle cell mutation (FIG. 11B). Increases in % HbG resulting from HDAC1/2 inhibition were due to increased HbG and decreased HbB (FIGS. 18B, 21D and 21E). HDAC1/2 inhibition also induced HbE and suppressed HbD, although their absolute levels accounted for less than 2% of total β-like globin transcripts. Suppression of the HbB and HbD and induction of HbE and HbG by Compound 001 is consistent with a globin switching model of HbG activation.

HbF protein levels were increased by over 3-fold upon treatment with 1 μM Compound 001 or Entinostat (FIG. 10C). HDAC inhibition also increased the mean fluorescent intensity (MFI) of the HbF positive cells, a measure of HbF abundance per cell, by up to 2-fold. The finding that Entinostat and Compound 001 show similar levels of HbG and HbF induction is consistent with the comparable HDAC1/2 potency of both compounds (FIG. 17B, FIG. 9A and FIG. 17D). This result also suggests that HDAC3 does not significantly contribute to HbG induction at the stages of differentiation in these culture systems.

### Example 26

### Differential Effects of HDAC Inhibition on Hematopoietic and Lineage-Specific Progenitors

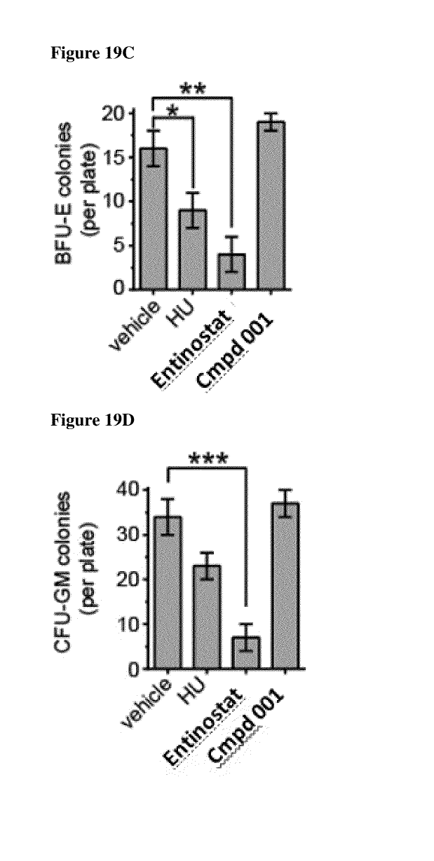

The biochemical and HbG induction assays described above demonstrated that Compound 001 and Entinostat have comparable ability to enter the cell, inhibit HDAC1/2, and elicit a pharmacodynamic response. These matched properties allowed investigation of the hypothesis that an HDAC1/2-selective inhibitor, such as Compound 001, is potentially less cytotoxic compared to an HDAC1/2/3-selective inhibitor, such as Entinostat. This possibility was first tested by measuring the viability of expanded human bone marrow-derived CD34+ cells, composed of primitive and more differentiated hematopoietic progenitors of multiple lineages, following 2 days of treatment with compound. Entinostat had an IC₅₀ value of 3 μM in these cells, while Compound 001 was 10-fold less cytotoxic with an IC₅₀ of 45 μM (FIG. 19A), suggesting that selective inhibition of HDAC1/2 with Compound 001 significantly reduces cytotoxicity to hematopoietic progenitors.

To identify the effects of HDAC inhibition on lineage-specific progenitors, 1 μM of Entinostat or Compound 001 were tested in colony formation assays. 1 μM Entinostat or 10 μM hydroxyurea resulted in a significant reduction in both the size and number of BFU-E colonies, while 1 μM Compound 001 had no effect (FIGS. 19B and 19C). Similarly, 1 μM Entinostat reduced the number of CFU-GM colonies, while 1 μM Compound 001 had no effect (FIG. 19D). 1 μM Entinostat and 1 μM Compound 001 had no effect on the number of CFU-E colonies, but significantly suppressed the number of CFU-MK colonies (FIG. 22). These findings suggest that inhibition of HDAC1/2/3, as opposed to HDAC1/2, results in greater cytotoxicity to early erythroid progenitors, as well as to granulocyte-monocyte progenitors. Furthermore, megakaryocyte progenitors appear particularly sensitive to the inhibition of HDAC1/2, a finding consistent with the phenotype of HDAC1 or 2 knockout mice.

To investigate the effects of HDAC inhibition on later stages of erythroid maturation, differentiation in CS1 was followed by flow cytometry using fluorescent antibodies against the transferrin receptor (CD71) and glycophorin A (GlyA). Cells treated with vehicle or 1 μM of Compound 001 or Entinostat differentiated normally over the first 5 days, becoming CD71^{pos}GlyA^{mid} (FIG. 19E). The majority of vehicle control cells continued to differentiate, becoming CD71^{pos}GlyA^{pos} by day 8. In contrast, cells treated with either HDAC inhibitor did not fully upregulate GlyA, but rather accumulated at the CD71^{pos}GlyA^{mid} stage. CD71^{pos}GlyA^{mid} cells are equivalent to proerythroblasts (ProE), while CD71^{pos}GlyA^{pos} cells include the more differentiated basophilic and polychromatic erythroblasts. Therefore, while HDAC1/2 inhibition using 1 μM of Compound 001 did not affect BFU-E and CFU-E colony number or size, this concentration of drug was able to block differentiation of proerythroblasts to basophilic erythroblasts in the liquid culture systems utilized in this study.

### Example 27

### Effect of HDAC1/2 Inhibition on Gene Expression

The mechanism through which HDAC1/2 inhibition induces HbG by performing gene expression profiling was interrogated. Because HDAC inhibition prevented cells from fully upregulating GlyA (FIG. 19E), RNA was isolated at day 5 of differentiation, a time point prior to the observed differentiation block. For each of 3 independent experiments, vehicle and Compound 001 treated cells showed similar CD71/GlyA differentiation profiles (FIGS. 1F and 1L), lending confidence that the resulting gene expression profiles were measuring a compound-specific effect that was not confounded by a shift in the maturity of cell populations. Using a filter of absolute fold change greater than 1.5 and a P-value less than 0.025, Compound 001 treatment was found to induce twice as many genes as it suppressed, 1294 and 681 respectively, a result consistent with the positive association of histone acetylation with chromatin accessibility and gene expression (FIG. 1G).

To determine if the gene expression changes resulting from Compound 001 treatment were similar to the gene expression changes resulting from HDAC1 or HDAC2 knockdown (KD), published gene expression data were analyzed for HDAC1 or HDAC2 knockdown in primary erythroblasts, and these gene sets were appended to a list of pre-existing gene sets. This collection of 2781 gene sets was queried against the Compound 001 and vehicle expression profiles. Robust and statistically significant enrichment was identified for the gene set ‘Up in HDAC2 KD’ (FIG. 1J), as well as for the gene sets ‘Up in HDAC1 KD’ and ‘Down in HDAC2 KD’ (FIG. 1M). As a measure of biological specificity, false discovery rate was plotted as a function of the gene set normalized enrichment score (FIG. 1K). ‘Up in HDAC1 KD’ and ‘Up in HDAC2 KD’ were the top two enriched gene sets in the Compound 001 expression profile. Taken together, these finding suggest that pharmacologic inhibition of HDAC1/2 recapitulates genetic ablation of HDAC1 or HDAC2.

Next, using the GeneChip data, a candidate gene approach was taken to determine which HbG modulators were changing as a result of both chemical and genetic HDAC1/2 inhibition (FIG. 1A). It was found that HbG repressors Bcl11a14 and Sox6, were down-regulated 1.3- and 2.5-fold by Compound 001 treatment, respectively. Bcl11a was also suppressed 2-fold by HDAC1 or HDAC2 KD. In contrast, other HbG repressors, such as Myb and Klf1 were unaffected. Expression changes were not observed for other genes involved in HbG regulation, including KDM1A (LSD1), NR2C1 (TR2) and NR2C2 (TR4), NR2F2 (COUP-TFII) and nuclear factor Y subunits, and proteins known to associate with Bcl11a12 (data not shown). However, Gata2, a proposed HbG and HbE activator, was up-regulated 2.8-fold by Compound 001 treatment and 1.5-fold by knockdown of HDAC1 or HDAC2 (FIG. 1A).

These observations were confirmed and extended using QPCR to measure temporal gene expression changes resulting from Compound 001 treatment (summarized in FIG. 1I). Gata1 increased and Gata2 decreased in control cells during the 5 days differentiation period, a result consistent with the known expression pattern of these genes during erythropoiesis (FIG. 1C). Gata1 and Klf1, master regulators of erythropoiesis, were unaffected by Compound 001 treatment (FIG. 1C and FIG. 1N), a finding consistent with CD71/GlyA profiles (FIG. 1F), further suggesting differentiation is unaffected by Compound 001 treatment prior to the basophilic erythroblast stage. In contrast, Compound 001 treatment prevented the suppression of Gata2, resulting in a 2-fold increase relative to control cells at day 2 and a 5-fold increase at day 5 (FIGS. 1B and C). Taken together, these results suggest that inhibition of HDAC1/2 prevents the suppression of Gata2 gene expression during normal erythroid maturation. Furthermore, unlike Bcl11a and Sox6 suppression, Gata2 induction by Compound 001 correlated with the timing of HbG induction (compare FIG. 10B with FIG. 1N), raising the possibilities that Gata2 may act as an HbG activator and Compound 001 may influence Gata2 gene regulation directly, possibly through altering histone acetylation at this locus.

### Example 28

### Gata2 Overexpression Induces HbG and Suppresses HbB

To test the hypothesis that Gata2 is an HbG activator, full length Gata2 or green fluorescent protein (GFP) were lentivirally delivered to expanded cells and then placed in differentiation media (FIG. 12A). In cells with ectopic Gata2 (oeG2), Gata2 mRNA and protein levels were 2.5-fold higher than GFP control cells (oeCtrl) throughout the differentiation period (FIG. 12B). Overexpression of Gata2 significantly increased the % HbG relative to control cells at day 3 and 5 of differentiation (FIG. 12D). Interrogation of each individual β-like globin transcript relative to actin revealed that the elevated % HbG in oeG2 cells resulted from increased HbG mRNA and decreased HbB mRNA (FIG. 12E). Gata2 overexpression also significant increased HbE mRNA.

Altering normal levels of Gata2 has the potential to affect erythroid differentiation, which could confound the interpretation of the finding above. Therefore, we measured cell surface levels of CD71 and GlyA by flow cytometry in oeCtrl and oeG2 cells at day 5 (FIG. 12C). We found their CD71/GlyA profiles to be highly similar, with the majority of cells upregulating both markers. As an additional indicator of erythroid differentiation stage, we measured the total β-like globin mRNA levels in control and Gata2 overexpressing cells. Consistent with the CD71/GlyA profiles, we found little difference in the total level of β-like globin mRNA at day 5 (FIG. 12F). Taken together, these data suggest that elevated Gata2 expression in erythroid progenitors is sufficient to induce HbG, without overtly affecting their maturation.

### Example 29

### Gata2 Knockdown Attenuates HbG Induction by Compound 001

To determine if Gata2 is necessary for HbG induction by Compound 001, shRNA targeting Gata2 (shG2-1 or shG2-2), or non-targeting control (shCtrl) were lentivirally delivered to cells (FIG. 13A). Since Gata2 levels decline during erythroid differentiation (FIGS. 1B and 1C), knockdown experiments were performed entirely in CS1 expansion media, which supports hematopoietic progenitors, and maintained Gata2 mRNA at a constant level in shCtrl cells following infection (FIG. 13B). Gata2 mRNA was reduced by 85% or 50% in shG2-1 or shG2-2 cells, respectively, while Gata2 protein was reduced by 50% by each hairpin, relative to shCtrl cells (FIG. 13B and FIG. 13D). Upon exposure to 1 uM Compound 001, cells expressing shCtrl induced Gata2 mRNA (1.4-fold) and Gata2 protein (5-fold) (FIG. 13B and FIG. 13D), that was coincident with a time-dependent induction of HbG mRNA (FIG. 13C). HbG mRNA was also induced by Compound 001 in shG2-1 and shG2-2 cells (FIG. 13C). However, the magnitude of this induction was reduced by 25% relative to shCtrl cells, indicating that reduced levels of Gata2 attenuates HbG induction by Compound 001, further supporting a role for Gata2 in HbG activation.

### Example 30

### HDAC1 and HDAC2 Co-Occupy the Gata2 Locus

To investigate how HDAC1/2 inhibition drives Gata2 activation, HDAC1 and 2 ChIP-seq experiments were performed on primary erythroid progenitors at a differentiation stage similar to the cells used for GeneChip experiments (FIG. 23A). It was found that HDAC1/2 are both highly abundant within a 15 kilobase (kb) region of the Gata2 locus (FIG. 14B, black histograms), and that this region is tightly correlated to HDAC1/2 binding in K562 cells from ENCODE (FIG. 14B, gray histograms). The strong correlation of HDAC1 and HDAC2 binding peaks suggests co-occupancy of this region. Furthermore, we observed that this region is bounded by the previously described +9.5 and −3.9 kb regulatory regions of the Gata2 gene, which are clearly identified by Gata2 binding peaks in K562 cells (FIG. 14B, deashed lines). This region of HDAC1/2 occupancy also includes the −2.8 kb and −1.8 kb Gata2 regulatory regions. Gata2 is known to be activated by a positive auto-regulatory loop in which Gata2 binding at the +9.5, −1.8, −2.8, and −3.9 kb regulatory regions plays a key role. The replacement of Gata2 by Gata1 at these regulatory regions, a process referred to as ‘gata switching,’ results in the suppression of Gata2 gene expression during early stages of erythroid cell maturation. Gata2 silencing is associated with a decrease in histone acetylation and chromatin accessibility at the +9.5, −1.8, −2.8, and −3.9 kb sites.

### Example 31

### Compound 001 Increases Histone Acetylation and Gata2 Binding at Gata2 Regulatory Regions

To test whether Compound 001-mediated inhibition of HDAC1/2 was leading to increased histone acetylation at the above-noted important regulatory regions, thereby promoting or prolonging Gata2 gene expression, primary erythroid progenitors were treated with vehicle or Compound 001 and used ChIP-QPCR to query levels of H2BK5ac, H3K9ac, and H3K27ac. In chromatin state maps these histone modifications, especially H3K9ac and H3K27ac, have been associated with active regulatory regions, such as enhancers and promoters of actively transcribed genes. The differentiation profile was unaffected by compound treatment and similar to that of cells used in experiments above (FIG. 23B). It was found that Compound 001 treatment led to significant increases histone acetylation at the +9.5, −1.8, −2.8, and −3.9 kb Gata2 regulatory regions, with maximum increases of 4- to 8-fold at the −1.8 kb region (FIG. 15B).

To see if the increases in histone acetylation were associated with increases in Gata2 binding, Gata2 ChIP-seq was performed in vehicle or Compound 001 treated cells. As above, differentiation profiles of compound treated cells were highly similar to control cells (FIG. 23A). It was observed that Gata2 occupancy at the Gata2 locus is tightly correlated between K562 and primary erythroid progenitors. In both cases, binding was limited to the +9.5, −1.8, −2.8, and −3.9 kb regulatory regions (data not shown). In response to Compound 001 treatment, Gata2 protein showed increased binding at all Gata2 regulatory regions, with a maximum increase in peak height of 3-fold at the −1.8 kb region. These experiments demonstrate that Compound 001 treatment results in elevated histone acetylation and Gata2 occupancy at Gata2 enhancer sites, suggesting that HDAC1/2 inhibition maintains the activity of the Gata2 autoregulatory loop, which is normally inactivated during erythroid maturation.

### Example 32

### Compound 001 Increases Gata2 Binding at a Region Near the HbD Promoter

The results described herein suggest that elevated Gata2 during erythropoiesis contributes to HbG induction, but the mechanism through which this occurs remains unknown. The tight correlation in the timing of Gata2 and HbG induction in response to Compound 001 treatment (compare FIG. 10B and FIGS. 1B and 1C), suggests that Gata2 may be acting directly on the β-like globin gene cluster. To investigate this possibility, the β-like globin gene cluster was looked at in the Gata2 ChIP-seq data described above. Six statistically significant Gata2 binding peaks were identified in vehicle and Compound 001 treated cells corresponding to four locus control region (LCR) hypersensitivity sites, a region in the HbB gene, and a region near the HbD gene promoter. Upon treatment with Compound 001, Gata2 binding increased 1.8-fold at the HbD promoter region, while the other regions were not affected (FIGS. 8A and 8B. An independent ChIP-QPCR experiment confirmed that Compound 001 treatment increases Gata2 binding by 2-fold at the HbD promoter, while a control region within the HbB gene was unaffected (FIG. 8C).

A role for the HbD promoter region in regulating HbG expression is supported by genetic studies; HbF levels in hemoglobinopathy patient samples correlated with the extent of HbD gene deletion, suggesting that regions 5′ to the HbD gene or the HbD gene itself were involved in HbG regulation, and, more recently, a region near the HbD promoter was identified as necessary for HbG repression. ENCODE data for K562 cells, in which HbG and HbE account for >99% of the β-like globin mRNA content, also suggest the HbD promoter region may contribute to HbG expression; it is marked as an active enhancer, a region of open chromatin, and a region of Gata2/Gata1 binding (data not shown), and Chromatin Conformation Capture Carbon Copy (5C) data shows that the LCR makes significant looping interactions with the HbD locus (data not shown). The HbD promoter region is co-occupied by Gata1, Sox6, Bcl11a, and the chromatin looping factor Lbd1/NLI. Since Gata2 and Gata1 compete for the same binding sites, it is plausible that elevated Gata2 may disrupt the recruitment of HbG repressors, or other regulatory factors, through displacement of Gata1 at the HbD promoter region.

### Example 33

### Gene Expression Profiling in MV4-11 AML Cell Line

MV4-11 cells were plated at 2×10⁵ cells/ml and treated with azacitidne at 1 μM, Compound 005 at 1 μM, Compound 005 at 2 μM, azacitidine at 1 μM plus Compound 005 at 1 μM, azacitidine at 1 μM plus Compound 005 at 2 μM for 24 h and 48 h. Cells were collected and RNA isolated. RNA samples were subjected to Affymetrix PrimeView Gene Expression profiling. Azacitidine at 1 μM and Compound 005 at 2 μM at 48 h were the focus of the initial data analysis. Molecular signatures were analyzed by GSEA (http://www.broadinstitute.org/gsea/index.jsp). The genes and signatures that were upregulated by the single and combination treatment are significantly more than those that were downregulated, consistent with the mechanisms of the compounds. In order to identify pathways and/or genes that mediate the combinatorial effects of azacitidine with Compound 005, signatures and genes that were upregulated by single agent and further upregulated by combination treatment were identified. Signatures including apoptosis and CEBPA pathway, a major transcription factor driving differentiation, are among the top pathways and/or genes identified. More than 60 genes including GATA2 and CD86 follow this expression pattern.

### Example 34

### Induction of GATA2 Expression in MV4-11 AML Cell Line

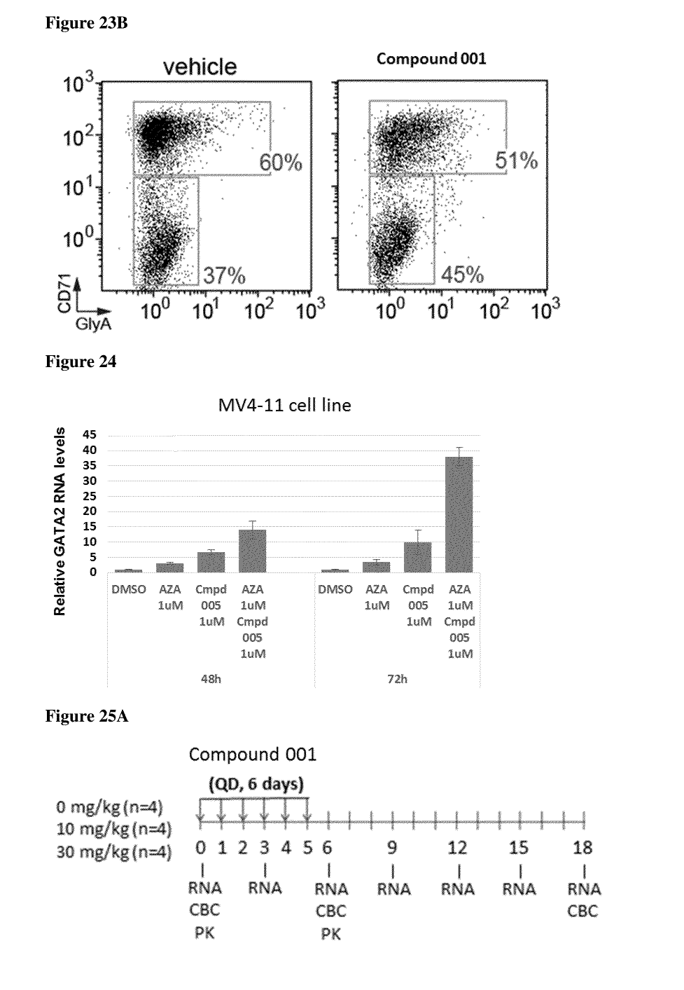

FIG. 24. Treatment of Compound 005 plus azacitidine significantly induced Gata2 in MV4-11 cells. (A-B) MV4-11 cells were plated at 2×10⁵ cells/ml at indicated doses for 48 h and 72 h. RNA was prepared and analyzed for GATA2 and GAPDH as internal control. Azacitidine at 1 uM and Compound 005 at 1 uM induced GATA2 level as single agent at 48 h and 72 h. Combination of azacitidine and Compound 005 further induced GATA2 expression at both time points.

### Example 35

### Experimental Design and Pharmacokinetics of Compound 001 in Rat and Cynomolgus Monkey

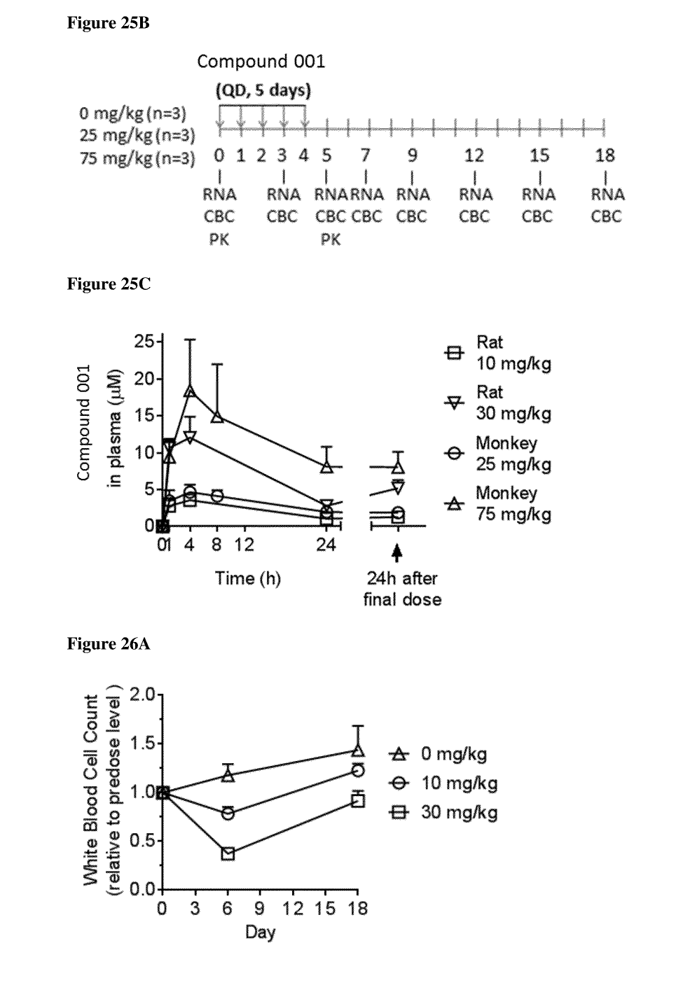

I. A. Rats were dosed once daily by oral gavage for 6 consecutive days at 0 mg/kg, 10 mg/kg, or 30 mg/kg per dose (See FIG. 25A). Dosing days are indicated by a down arrow (FIG. 25A). Peripheral blood was drawn on the indicated days and analyzed for drug level in plasma (PK), complete blood counts (CBC), or isolation of total ribonucleic acid (RNA).

B. Cynomolgus monkey were dosed once daily by oral gavage for 5 consecutive days at 0 mg/kg, 25 mg/kg, or 75 mg/kg per dose (FIG. 25B). Sampling as described in ‘A’.

C. Compound 001 levels in peripheral blood were measured during the 24 hours following the first dose of Compound 001 and at a single point 24 hours following the last dose of Compound 001 for the experiments described in ‘A’ and ‘B’. Points are the average of replicate animals, error bars are the standard deviation of replicate animals (FIG. 25C). FIG. 25C shows that the low dose animals had a minimum of 1 uM of drug exposure for the entire dosing period, while the high dose group maintained a minimum of 5 uM.

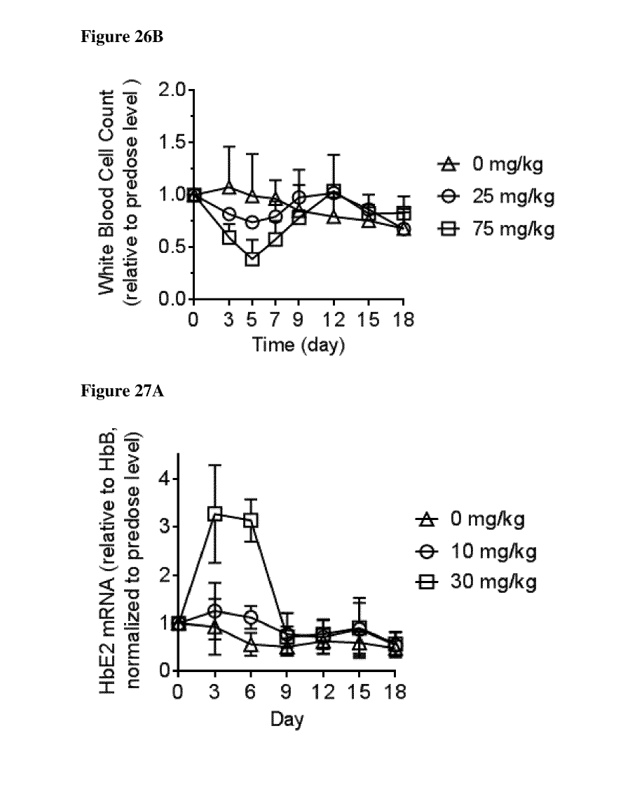

II. White blood cell counts from the experiments described above, Section I of this Example, (see FIGS. 25A-C) were measured (FIGS. 26A and 26B).

A. White blood cell counts were measured in the rats treated with Compound 001 in Section IA (see FIGS. 25A and 26B). For each individual rat, white blood cell counts were expressed relative to their predose level (time=day 0). Points are the average of n=4 animals with error bars showing the standard deviation.

B. White blood cell counts were measured in the monkeys treated with Compound 001 in Section IB (see FIGS. 25B and 26B). For each individual monkey, white blood cell counts were expressed relative to their predose level (time=day 0). Points are the average of n=3 animals with error bars showing the standard deviation.

There was no detected affect on RBC or platelets. These data indicate treatment with Compound 001 induces reversible suppression of white blood cells in both animal models, with peak suppression following 1 day after administration of the last dose of Compound 001. White blood cell counts recovered to baseline in both animal models 5 days after administration of the last dose of Compound 001.

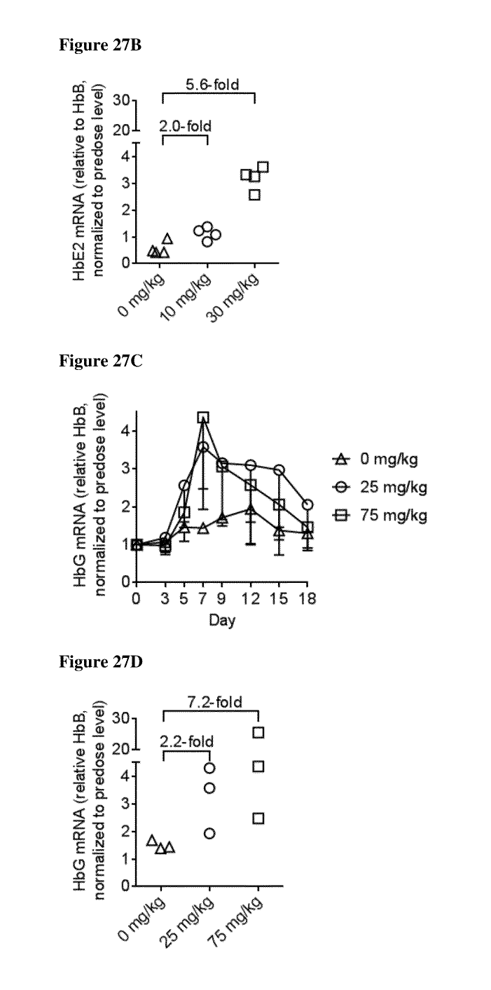

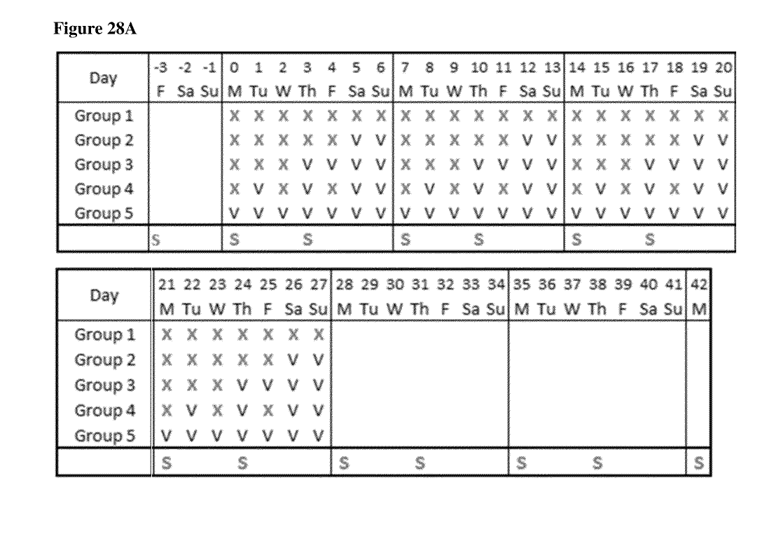

III. HbE and HbG induction by Compound 001 from the experiments described in Section I of this Example (see FIGS. 25A-C) were measured (FIGS. 27A-D).

HbE mRNA levels were measured in the rats treated with Compound 001 (see FIGS. 25A and 27A). For each individual rat, HbE mRNA was expressed relative to HbB mRNA and then normalized to their predose level (time=day 0). Points are the average of n=4 animals with error bars showing the standard deviation (FIG. 27A). HbE mRNA levels for each individual animal at day 6 are shown in FIG. 27B. This data shows a dose-dependent increase in HbE.

HbG mRNA levels were measured in the monkey treated with Compound 001 as described in Section I of this Example (see FIGS. 25B and 27C). For each individual monkey, HbG mRNA was expressed relative to HbB mRNA and then normalized to their predose level (time=day 0). Points are the median of n=3 animals with error bars showing the range. HbG mRNA levels for each individual animal at day 7 are shown in FIG. 27D. This data shows a dose-dependent increase in HbG.

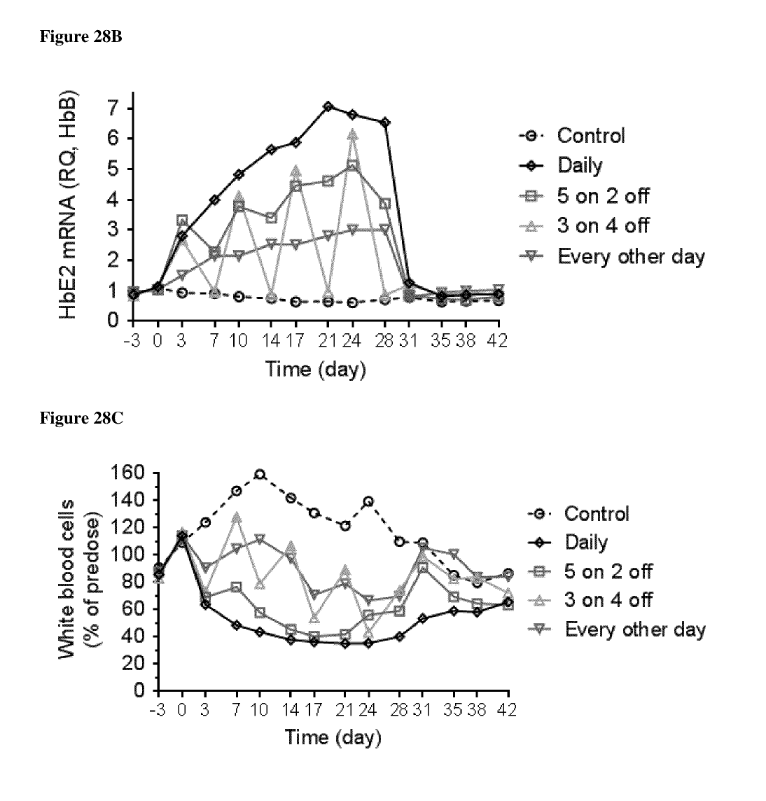

IV. Rats were dosed with 30 mg/kg once daily by oral gavage according to the above schedule. X=30 mg/kg Compound 001 dosing, V=vehicle control dosing, S=sampling of peripheral blood for complete blood counts and RNA isolation. Group 1=daily dosing, Group 2=5 on 2 off, Group 3=3 on 4 off, Group 4=every other day, Group 5=control. N=4 animals per group (FIG. 28A).

FIG. 28B shows the effect of dosing schedule on embryonic globin (HbE2) mRNA induction in peripheral blood. Rats were dosed with Compound 001 as described in FIG. 28A. Data points are the average of N=4 animals. HbE2 mRNA level was determined by quantitative real time PCR and expressed relative to HbB mRNA control. Data for each animal was then normalized to the expression level prior to the onset of dosing (average of day −3 and day 0 values).

FIG. 28C shows the effect of dosing schedule on white blood cell counts in peripheral blood. Rats were dosed with Compound 001 as described in FIG. 28A. Data points are the average of N=4 animals.

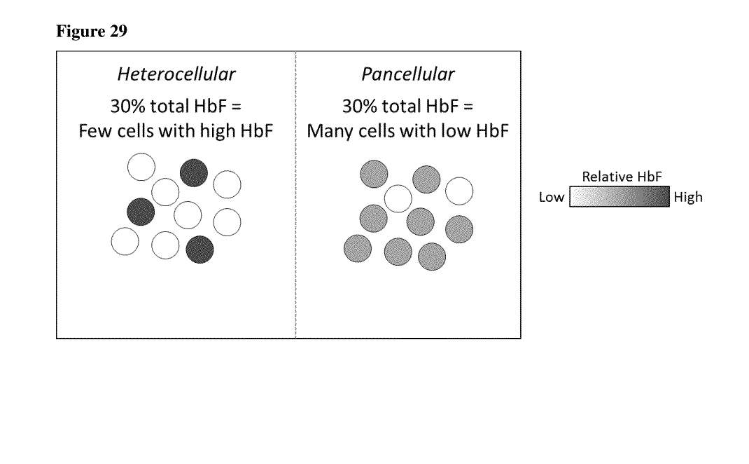

Correction of sickle cell disease has been hypothesized to require pancellular HbF expression and a total HbF of 30% (Steinberg M H, Chui D H, Dover G J, Sebastiani P, Alsultan A “Fetal hemoglobin in sickle cell anemia: a glass half full?” Blood, 2014, 123(4):481-5). Accordingly, one therapeutic goal is pancellular HbF of 30% with minimal mylosuppression (FIG. 29), as opposed to heterocellular HbF of 30% with greater mylosuppression.

The data described in FIGS. 28A-C suggest that dosing and scheduling play a role in meeting this therapeutic goal. These data show that Compound 001 dosing schedules result in dramatically different patterns of HBE induction and myelo suppression in the peripheral blood of Rat (FIGS. 28A-C). A 3 on 4 off dosing schedule suggests a heterocellular mode of HbF expression (FIG. 28B) associated with greater myelo suppression (FIG. 28C). Interestingly, an every other day dosing schedule suggests a pancellular mode of HbF expression (FIG. 28B) associated with less myelosuppression (FIG. 28C).

### INCORPORATION BY REFERENCE

The contents of all references (including literature references, issued patents, published patent applications, and co-pending patent applications) cited throughout this application are hereby expressly incorporated herein by reference in their entireties. Unless otherwise defined, all technical and scientific terms used herein are accorded the meaning commonly known to one with ordinary skill in the art.

### EQUIVALENTS

Those skilled in the art will recognize, or be able to ascertain using no more than routine experimentation, many equivalents of the specific embodiments provided herein. Such equivalents are intended with be encompassed by the following claims.

1. A method for treating a disease or disorder associated with GATA binding protein 2 (Gata2) deficiency comprising administering to a subject in need thereof a therapeutically effective amount of a compound of Formula I, II, III, IV, V, VI, VII, VIII, IX, X, or any of the compounds presented in Table 1, Table 2, Table 3, or Table 4, or a pharmaceutically salt thereof.

2. The method of claim 1, wherein the disease or disorder is selected from the group consisting of: acute myeloid leukemia, familial myelodysplastic syndrome, leukemia, sickle-cell anemia, and beta-thalassemia.

3. The method of claim 1, wherein the compound has the chemical structure of Formula I: 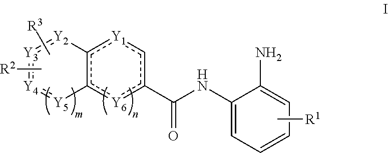
or a pharmaceutically acceptable salt thereof,
wherein
Y₁ is CR⁷ or NR⁷;
Y₂, Y₃, Y₄, Y₅, and Y₆ are each independently CH, CH₂, N, or C(O), wherein at least one of Y₂, Y₃, Y₄, and Y₅ are CH;
R¹ is mono-, bi-, or tri-cyclic aryl or heteroaryl, wherein the mono-, bi-, or tri-cyclic aryl or heteroaryl is optionally substituted with one or more groups selected from halo, C₁₋₄-alkyl, CO₂R⁶, C(O)R⁶, or C₁₋₆-alkyl-OR⁶;
R² and R³ are each independently selected from C₂₋₆-alkenyl, C₂₋₆-alkynyl, C₃₋₆-cycloalkyl, C₁₋₆-alkyl-C₃₋₆-cycloalkyl, heterocycloalkyl, C₁₋₆-alkyl-heterocycloalkyl, NR⁴R⁵, O—C₁₋₆-alkyl-OR⁶, C₁₋₆-alkyl-OR⁶, aryl, heteroaryl, C(O)N(H)-heteroaryl, C(O)-heteroaryl, C(O)-heterocycloalkyl, C(O)-aryl, C(O)—C₁₋₆-alkyl, CO₂—C₁₋₆-alkyl, or C(O)—C₁₋₆-alkyl-heterocycloalkyl, wherein the cycloalkyl, heterocycloalkyl, aryl, or heteroaryl is optionally, independently substituted one or more times with C₁₋₄-alkyl, CO₂R⁶, C(O)R⁶, or C₁₋₆-alkyl-OR⁶;
R⁴ is H, C₁₋₆-alkyl, or C₁₋₆-alkyl-OR⁶;
R⁵ is CO₂R⁶, C₁-C₆-alkyl-aryl, or C₁₋₆-alkyl-OR⁶;
R⁶ is H or C₁₋₆-alkyl;
R⁷ is null, H, C₁₋₆-alkyl, C₃₋₆-cycloalkyl, C₁₋₆-alkyl-C₃₋₆-cyclo alkyl, heterocycloalkyl, or C₁₋₆-alkyl-heterocycloalkyl;
a [symbol] line denotes an optionally double bond;
m is 0 or 1; and
n is 0 or 1, provided at least one of m or n is 1.

4. The method of claim 3, wherein
R¹ is mono-, bi-, or tri-cyclic aryl or heteroaryl, wherein the mono-, bi-, or tri-cyclic aryl or heteroaryl is optionally substituted with halo, C₁₋₄-alkyl, CO₂R⁶, C(O)R⁶, or C₁₋₆-alkyl-OR⁶; and
R² and R³ are each independently selected from C₂₋₆-alkenyl, C₂₋₆-alkynyl, C₃₋₆-cycloalkyl, C₁₋₆-alkyl-C₃₋₆-cycloalkyl, heterocycloalkyl, C₁₋₆-alkyl-heterocycloalkyl, NR⁴R⁵, O—C₁₋₆-alkyl-OR⁶, C₁₋₆-alkyl-OR⁶, aryl, heteroaryl, C(O)N(H)-heteroaryl, C(O)-heteroaryl, C(O)-heterocycloalkyl, C(O)-aryl, C(O)—C₁₋₆-alkyl, CO₂—C₁₋₆-alkyl, and C(O)—C₁₋₆-alkyl-heterocycloalkyl, wherein the cycloalkyl, heterocycloalkyl, aryl, or heteroaryl is optionally, independently substituted one or more times with C₁₋₄-alkyl, CO₂R⁶, C(O)R⁶, or C₁₋₆-alkyl-OR⁶.

5. The method of claim 3, wherein
R¹ is monocyclic aryl or heteroaryl, wherein the aryl or heteroaryl is optionally substituted with halo;
R² and R³ are each independently selected from C₂₋₆-alkenyl, C₃₋₆-cycloalkyl, C₁₋₆-alkyl-C₃₋₆-cycloalkyl, heterocycloalkyl, C₁₋₆-alkyl-heterocycloalkyl, NR⁴R⁵, O—C₁₋₆-alkyl-OR⁶, or C₁₋₆-alkyl-OR⁶;
R⁴ is H or C₁₋₆-alkyl;
R⁵ is CO₂R⁶ or C₁₋₆-alkyl-OR⁶; and
R⁶ is C₁₋₆-alkyl.

6. The method of claim 3, wherein
m is 1; n is 1; Y₁ is N; and Y₂, Y₃, Y₄, Y₅, and Y₆ are each CH;
m is 0; n is 1; Y₂ is N; Y₁ is CR⁷; and Y₃, Y₄, and Y₆ are each CH;
m is 0; n is 1; Y₁ is CR⁷; Y₂ is N; Y₃ is C(O); Y₄ is CH₂; and Y₆ is CH;
m is 1; n is 1; Y₁ is CR⁷; Y₂ is N, and Y₃, Y₄, Y₅, and Y₆ are each CH;
m is 0; n is 1; Y₁ is CR⁷; Y₂ and Y₃ are each N; and Y₄ and Y₆ are each CH;
m is 0; n is 1; Y₁ and Y₂ are N; Y₃, Y₄, and Y₆ are each CH; or
m is 1; n is 1; and Y1, Y₂, Y₃, Y₄, Y₅, and Y₆ are each CH.

7. The method of claim 3, wherein the compound of Formula I is selected from the group consisting of: 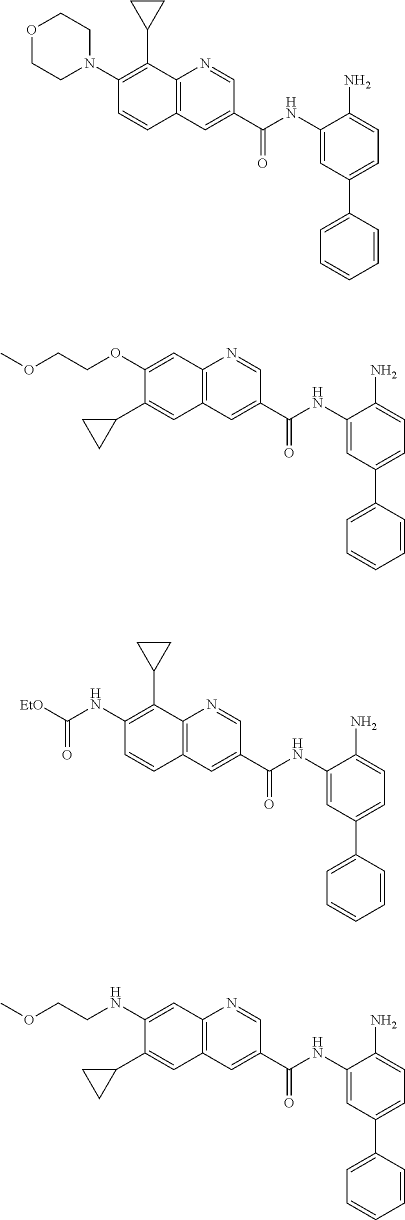 [SMILES: Nc1ccc(-c2ccccc2)cc1NC(=O)c1cnc2c(C3CC3)c(N3CCOCC3)ccc2c1 ; COCCOc1cc2ncc(C(=O)Nc3cc(-c4ccccc4)ccc3N)cc2cc1C1CC1 ; CCOC(=O)Nc1ccc2cc(C(=O)Nc3cc(-c4ccccc4)ccc3N)cnc2c1C1CC1 ; COCCNc1cc2ncc(C(=O)Nc3cc(-c4ccccc4)ccc3N)cc2cc1C1CC1] 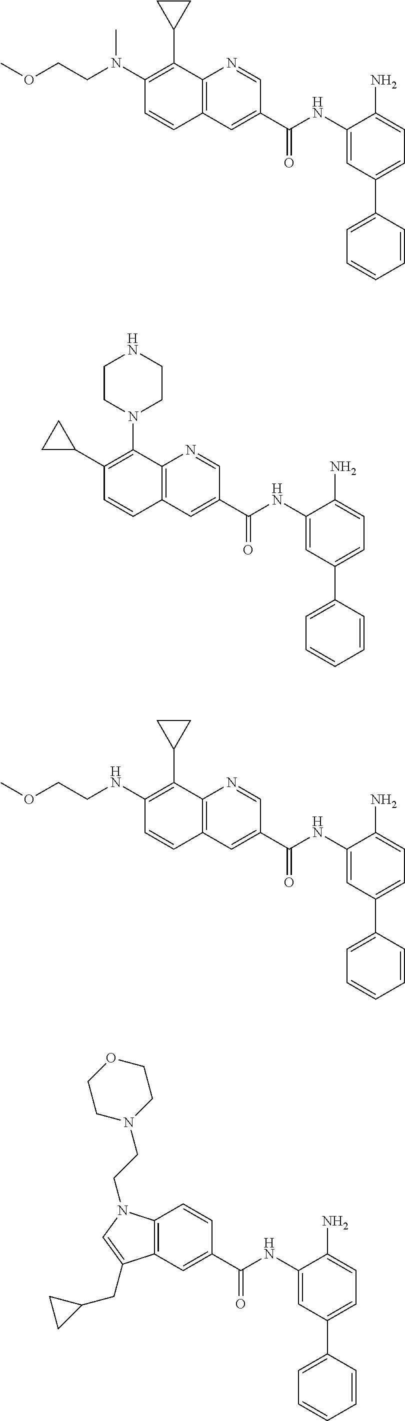 [SMILES: COCCN(C)c1ccc2cc(C(=O)Nc3cc(-c4ccccc4)ccc3N)cnc2c1C1CC1 ; Nc1ccc(-c2ccccc2)cc1NC(=O)c1cnc2c(N3CCNCC3)c(C3CC3)ccc2c1 ; COCCNc1ccc2cc(C(=O)Nc3cc(-c4ccccc4)ccc3N)cnc2c1C1CC1 ; Nc1ccc(-c2ccccc2)cc1NC(=O)c1ccc2c(c1)c(CC1CC1)cn2CCN1CCOCC1] 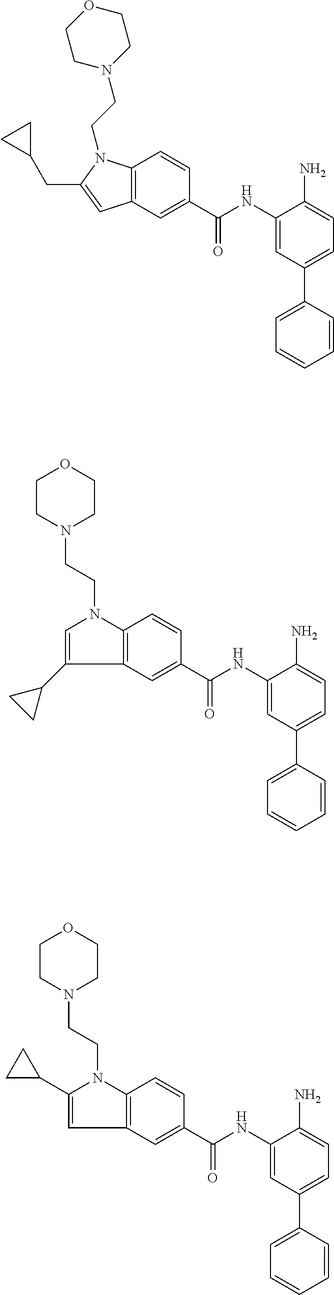 [SMILES: Nc1ccc(-c2ccccc2)cc1NC(=O)c1ccc2c(c1)cc(CC1CC1)n2CCN1CCOCC1 ; Nc1ccc(-c2ccccc2)cc1NC(=O)c1ccc2c(c1)c(C1CC1)cn2CCN1CCOCC1 ; Nc1ccc(-c2ccccc2)cc1NC(=O)c1ccc2c(c1)cc(C1CC1)n2CCN1CCOCC1] 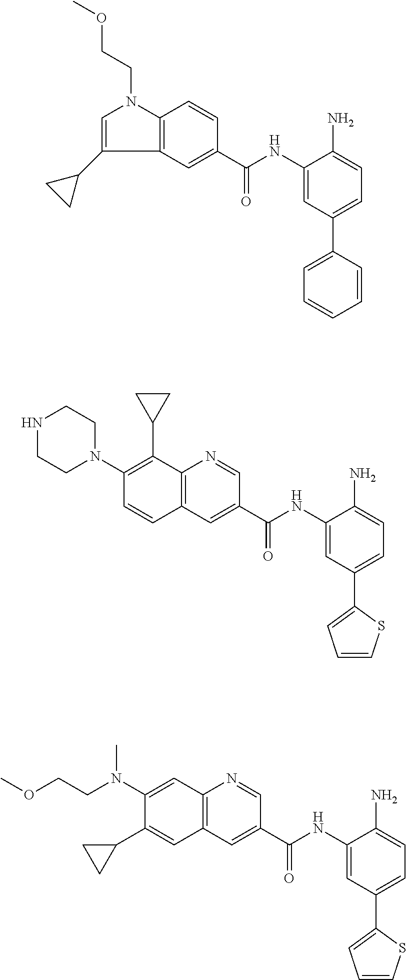 [SMILES: COCCn1cc(C2CC2)c2cc(C(=O)Nc3cc(-c4ccccc4)ccc3N)ccc21 ; Nc1ccc(-c2cccs2)cc1NC(=O)c1cnc2c(C3CC3)c(N3CCNCC3)ccc2c1 ; COCCN(C)c1cc2ncc(C(=O)Nc3cc(-c4cccs4)ccc3N)cc2cc1C1CC1] 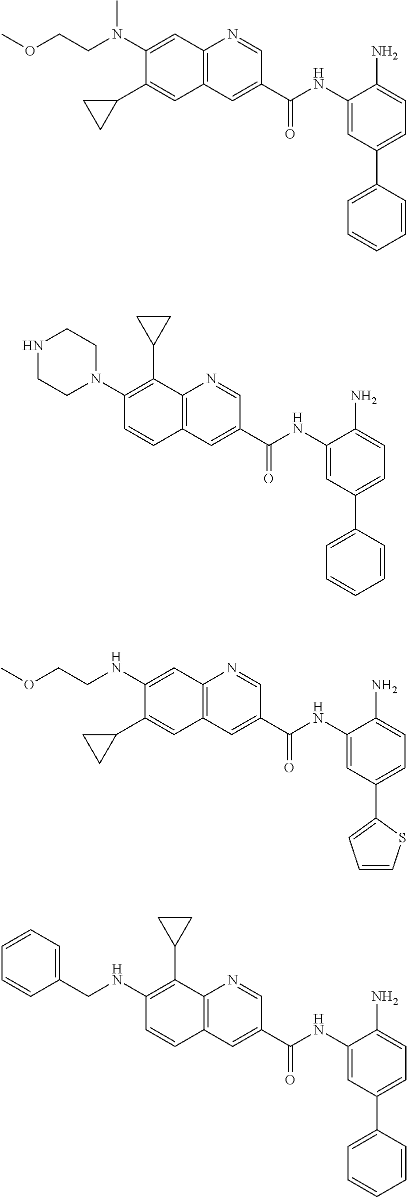 [SMILES: COCCN(C)c1cc2ncc(C(=O)Nc3cc(-c4ccccc4)ccc3N)cc2cc1C1CC1 ; Nc1ccc(-c2ccccc2)cc1NC(=O)c1cnc2c(C3CC3)c(N3CCNCC3)ccc2c1 ; COCCNc1cc2ncc(C(=O)Nc3cc(-c4cccs4)ccc3N)cc2cc1C1CC1 ; Nc1ccc(-c2ccccc2)cc1NC(=O)c1cnc2c(C3CC3)c(NCc3ccccc3)ccc2c1] 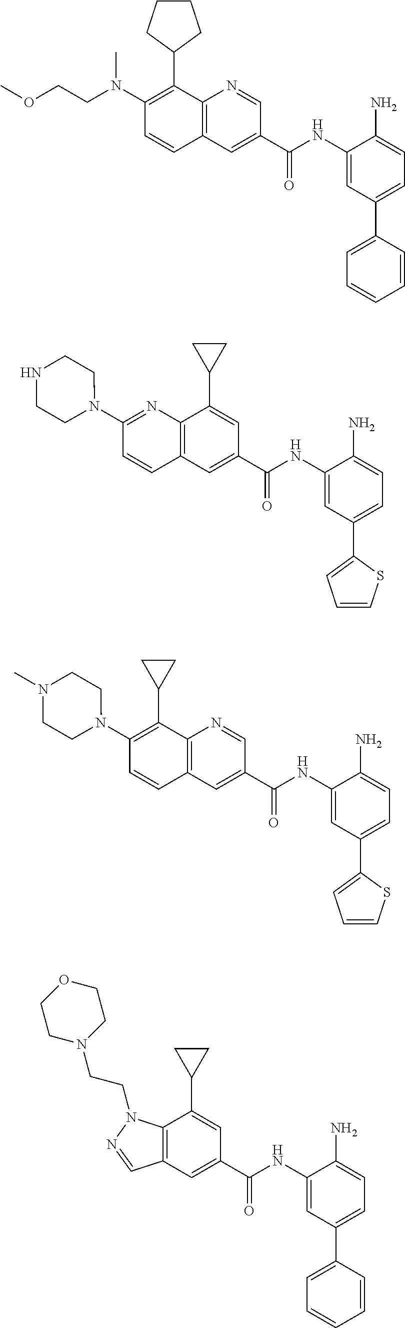 [SMILES: COCCN(C)c1ccc2cc(C(=O)Nc3cc(-c4ccccc4)ccc3N)cnc2c1C1CCCC1 ; Nc1ccc(-c2cccs2)cc1NC(=O)c1cc(C2CC2)c2nc(N3CCNCC3)ccc2c1 ; CN1CCN(c2ccc3cc(C(=O)Nc4cc(-c5cccs5)ccc4N)cnc3c2C2CC2)CC1 ; Nc1ccc(-c2ccccc2)cc1NC(=O)c1cc(C2CC2)c2c(cnn2CCN2CCOCC2)c1] 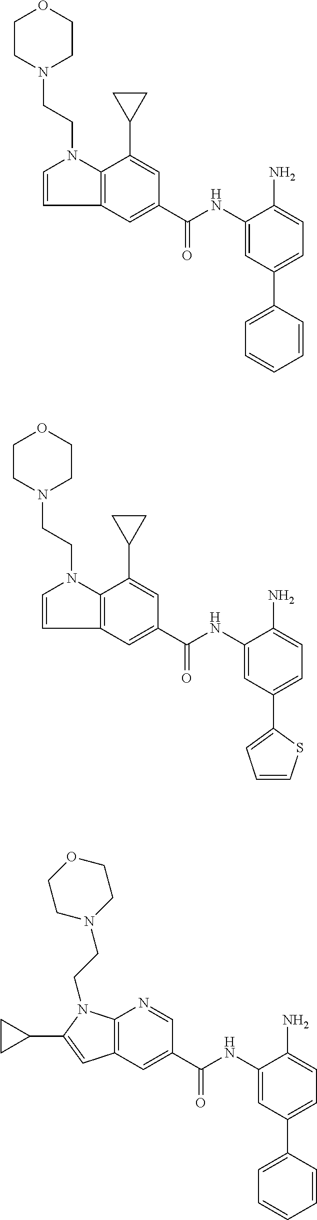 [SMILES: Nc1ccc(-c2ccccc2)cc1NC(=O)c1cc(C2CC2)c2c(ccn2CCN2CCOCC2)c1 ; Nc1ccc(-c2cccs2)cc1NC(=O)c1cc(C2CC2)c2c(ccn2CCN2CCOCC2)c1 ; Nc1ccc(-c2ccccc2)cc1NC(=O)c1cnc2c(c1)cc(C1CC1)n2CCN1CCOCC1] 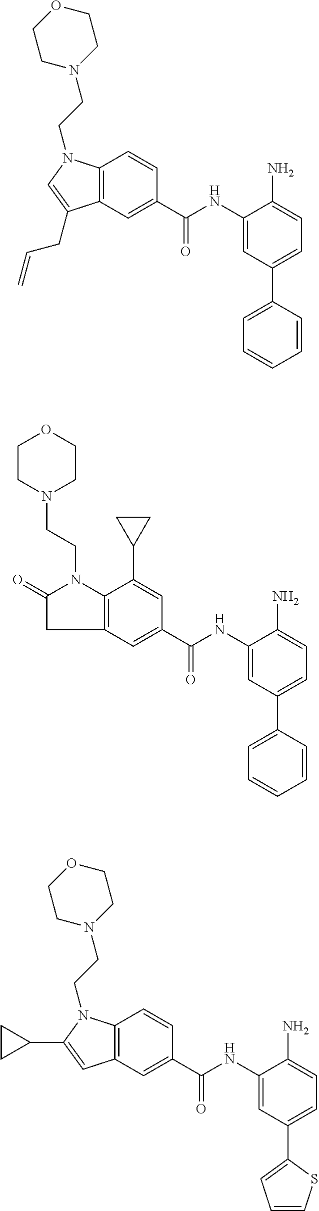 [SMILES: C=CCc1cn(CCN2CCOCC2)c2ccc(C(=O)Nc3cc(-c4ccccc4)ccc3N)cc12 ; Nc1ccc(-c2ccccc2)cc1NC(=O)c1cc2c(c(C3CC3)c1)N(CCN1CCOCC1)C(=O)C2 ; Nc1ccc(-c2cccs2)cc1NC(=O)c1ccc2c(c1)cc(C1CC1)n2CCN1CCOCC1] 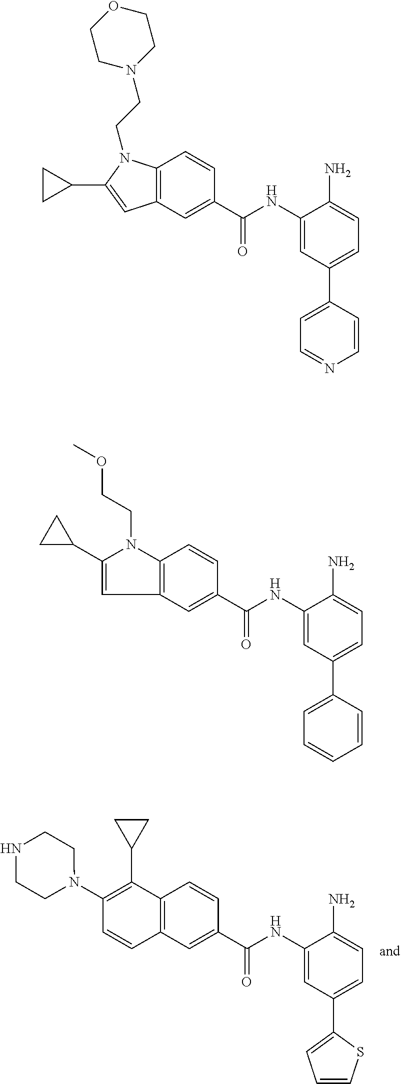 [SMILES: Nc1ccc(-c2ccncc2)cc1NC(=O)c1ccc2c(c1)cc(C1CC1)n2CCN1CCOCC1 ; COCCn1c(C2CC2)cc2cc(C(=O)Nc3cc(-c4ccccc4)ccc3N)ccc21 ; Nc1ccc(-c2cccs2)cc1NC(=O)c1ccc2c(C3CC3)c(N3CCNCC3)ccc2c1] 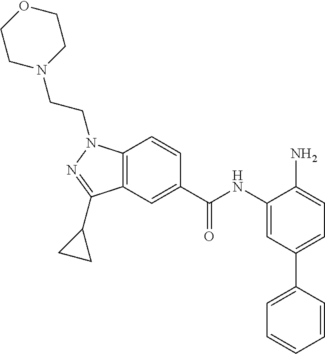 [SMILES: Nc1ccc(-c2ccccc2)cc1NC(=O)c1ccc2c(c1)c(C1CC1)nn2CCN1CCOCC1]
or a pharmaceutically acceptable salt thereof.

8. The method of claim 1, wherein the compound has the chemical structure of Formula II: 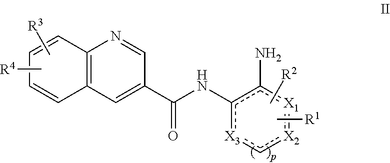
or a pharmaceutically acceptable salt thereof;
wherein
R¹ and R² are independently H, mono-, bi-, or tri-cyclic aryl or heteroaryl, wherein the mono-, bi-, or tri-cyclic aryl or heteroaryl is optionally substituted with halo, C₁₋₄-alkyl, CO₂R⁷, C(O)R⁷, or C₁₋₆-alkyl-OR⁷;
or R¹ and R² are linked together to form a group of Formula: 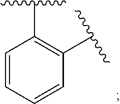 [SMILES: *c1ccccc1*]
R³ and R⁴ are independently selected from H, C₁₋₆-alkyl, C₂₋₆-alkenyl, C₂₋₆-alkynyl, C₃₋₆-cycloalkyl, C₁₋₆-alkyl-C₃₋₆-cycloalkyl, heterocycloalkyl, C₁₋₆-alkyl-heterocycloalkyl, NR⁵R⁶, O—C₁₋₆-alkyl-OR⁷, aryl, heteroaryl, C(O)N(H)-heteroaryl, C(O)-heteroaryl, C(O)-heterocycloalkyl, C(O)-aryl, C(O)—C₁₋₆-alkyl, CO₂—C₁₋₆-alkyl, or C(O)—C₁₋₆-alkyl-heterocycloalkyl, wherein the cycloalkyl, heterocycloalkyl, aryl, or heteroaryl is optionally substituted with halo, C₁₋₄-alkyl, CO₂R⁷, C(O)R⁷, or C₁₋₆-alkyl-OR⁷;
R⁵ is H, C₁₋₆-alkyl, CO₂R⁷ or C₁₋₆-alkyl-OR⁷;
R⁶ is H, C₁₋₆-alkyl, CO₂R⁷ or C₁₋₆-alkyl-OR⁷;
R⁷ is H or C₁₋₆-alkyl;
X₁, X₂, and X₃ are each independently CH, N, or S, wherein at least one of X₁ or X₂ is N or S;
a [symbol] line denotes an optionally double bond; and
p is 0 or 1.

9. The method of claim 8, wherein
R¹ is mono-, bi-, or tri-cyclic aryl or heteroaryl, wherein the mono-, bi-, or tri-cyclic aryl or heteroaryl is optionally substituted with halo, C₁₋₄-alkyl, CO₂R⁷, C(O)R⁷, or C₁₋₆-alkyl-OR⁷;
R² is H;
or R¹ and R² are linked together to form the following fused ring: 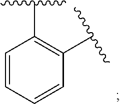 [SMILES: *c1ccccc1*]
and
R³ and R⁴ are independently selected from H, C₁₋₆-alkyl, C₂₋₆-alkenyl, C₂₋₆-alkynyl, C₃₋₆-cycloalkyl, C₁₋₆-alkyl-C₃₋₆-cycloalkyl, heterocycloalkyl, C₁₋₆-alkyl-heterocycloalkyl, NR⁵R⁶, O—C₁₋₆-alkyl-OR⁷, aryl, heteroaryl, C(O)N(H)-heteroaryl, C(O)-heteroaryl, C(O)-heterocycloalkyl, C(O)-aryl, C(O)—C₁₋₆-alkyl, CO₂—C₁₋₆-alkyl, or C(O)—C₁₋₆-alkyl-heterocycloalkyl.

10. (canceled)

11. The method of claim 1, wherein the compound of Table 1 is selected from the group consisting of: 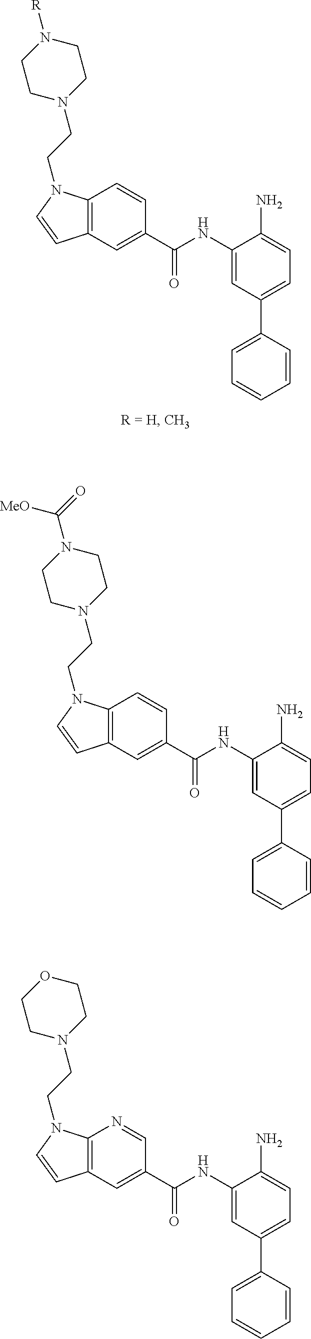 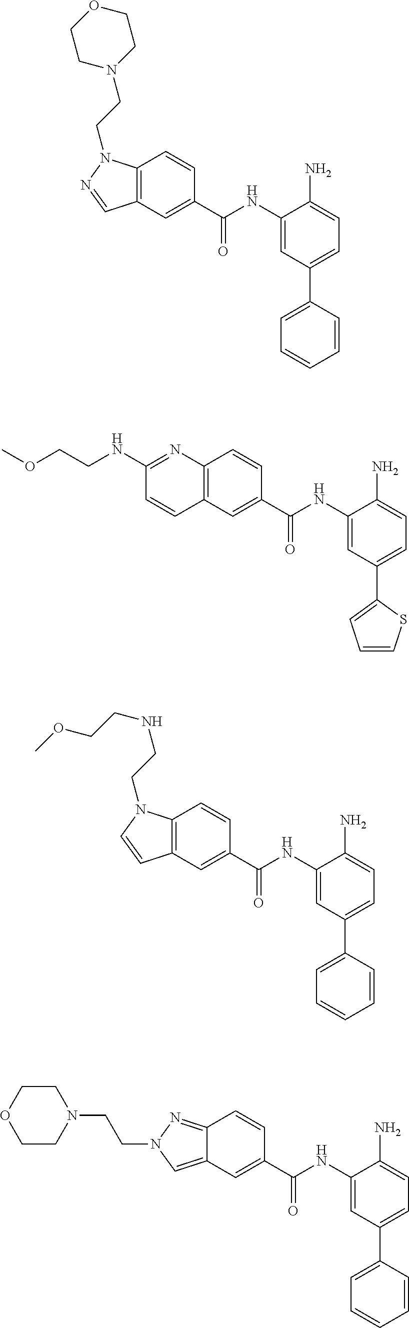 [SMILES: Nc1ccc(-c2ccccc2)cc1NC(=O)c1ccc2c(cnn2CCN2CCOCC2)c1 ; COCCNc1ccc2cc(C(=O)Nc3cc(-c4cccs4)ccc3N)ccc2n1 ; COCCNCCn1ccc2cc(C(=O)Nc3cc(-c4ccccc4)ccc3N)ccc21 ; Nc1ccc(-c2ccccc2)cc1NC(=O)c1ccc2nn(CCN3CCOCC3)cc2c1] 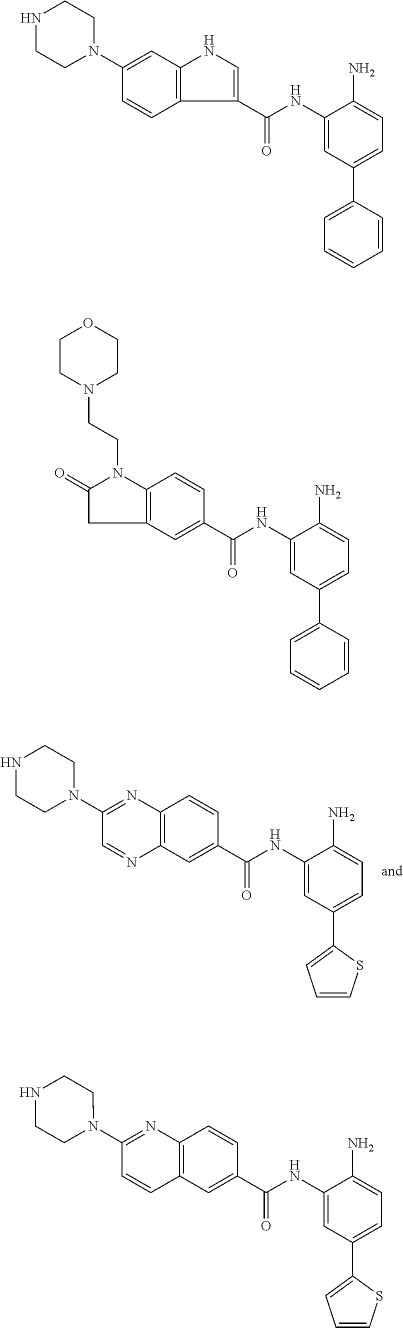 [SMILES: Nc1ccc(-c2ccccc2)cc1NC(=O)c1c[nH]c2cc(N3CCNCC3)ccc12 ; Nc1ccc(-c2ccccc2)cc1NC(=O)c1ccc2c(c1)CC(=O)N2CCN1CCOCC1 ; Nc1ccc(-c2cccs2)cc1NC(=O)c1ccc2nc(N3CCNCC3)cnc2c1 ; Nc1ccc(-c2cccs2)cc1NC(=O)c1ccc2nc(N3CCNCC3)ccc2c1]
or a pharmaceutically acceptable salt thereof.

12. The method of claim 1, wherein the compound has the chemical structure of Formula IV: 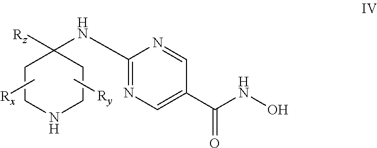
or a pharmaceutically acceptable salt thereof,
wherein,
Rₓ is selected from the group consisting of C₁₋₆-alkyl, C₁₋₆-alkoxy, halo, —OH, —C(O)R¹, —CO₂R¹, —C(O)N(R¹)₂, aryl, —C(S)N(R¹)₂, and S(O)₂R¹, wherein aryl is optionally substituted with one or more groups selected from C₁₋₆-alkyl, C₁₋₆-alkoxy, —OH, halo, and haloalkyl;
R_{y} is selected from the group consisting of H, C₁₋₆-alkyl, C₁₋₆-alkoxy, halo, —OH, —C(O)R¹, —CO₂R¹, and —C(O)N(R¹)₂;
R_{z} is selected from the group consisting of C₁₋₆-alkyl, C₁₋₆-alkenyl, C₁₋₆-alkynyl, C₃₋₈-cycloalkyl, C₃₋₇-heterocycloalkyl, aryl, and heteroaryl, each of which is optionally substituted with C₁₋₆-alkyl, C₁₋₆-alkoxy, halo, or —OH; and
each R¹ is, independently for each occurrence, selected from the group consisting of H, C₁₋₆-alkyl, C₃₋₈-cycloalkyl, C₃₋₇-heterocycloalkyl, aryl, heteroaryl, C₁₋₆-alkyl-cycloalkyl, C₁₋₆-alkyl-heterocycloalkyl, C₁₋₆-alkyl-aryl, and C₁₋₆-alkyl-heteroaryl, wherein C₃₋₈-cycloalkyl, C₃₋₇-heterocycloalkyl, aryl, heteroaryl, C₁₋₆-alkyl-cycloalkyl, C₁₋₆-alkyl-heterocycloalkyl, C₁₋₆-alkyl-aryl, and C₁₋₆-alkyl-heteroaryl is optionally substituted with one or more groups selected from C₁₋₆-alkyl, C₁₋₆-alkoxy, —OH, halo, and haloalkyl.

13. The method of claim 12, wherein the compound has the structure of Formula V: 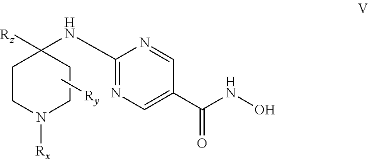
or a pharmaceutically acceptable salt thereof,
wherein,
Rₓ is independently selected from the group consisting of aryl, —C(O)R′, —CO₂R¹, —C(O)N(R¹)₂, —C(S)N(R¹)₂, and S(O)₂R¹;
R_{y} is selected from the group consisting of H, C₁₋₆-alkyl, or, halo; and
R_{z} is selected from the group consisting of C₁₋₆-alkyl, C₃₋₈-cycloalkyl, C₃₋₇-heterocycloalkyl, aryl, and heteroaryl.

14. The method of claim 12, wherein the compound is selected from the group consisting of: 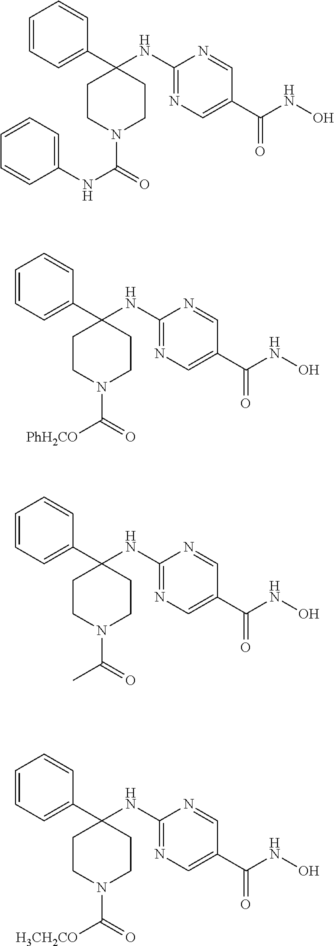 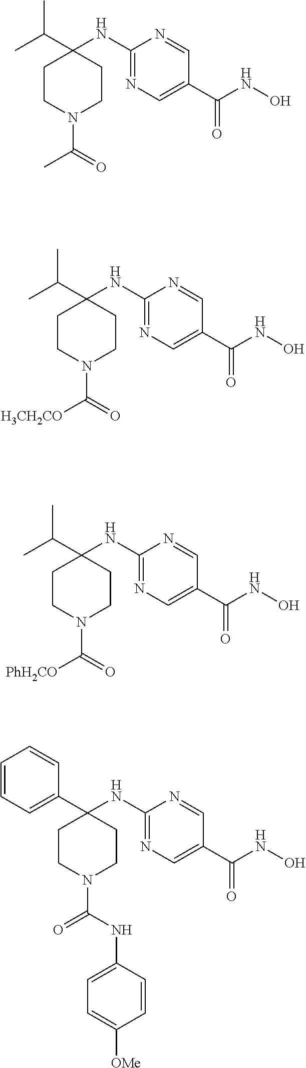 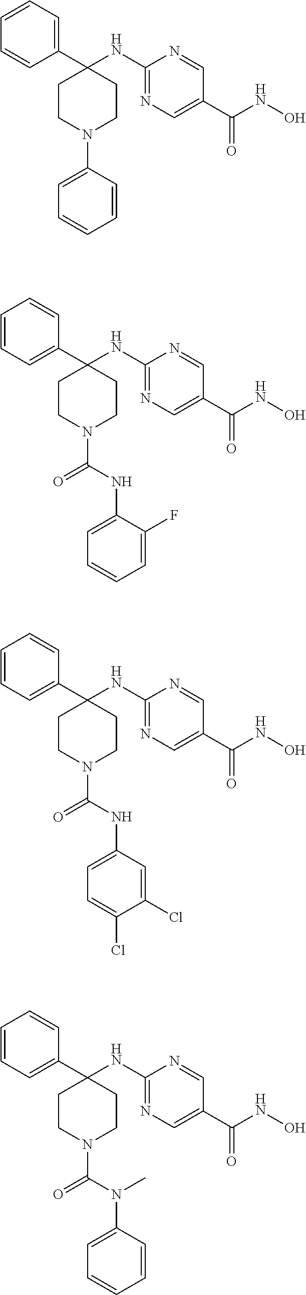 [SMILES: O=C(NO)c1cnc(NC2(c3ccccc3)CCN(c3ccccc3)CC2)nc1 ; O=C(NO)c1cnc(NC2(c3ccccc3)CCN(C(=O)Nc3ccccc3F)CC2)nc1 ; O=C(NO)c1cnc(NC2(c3ccccc3)CCN(C(=O)Nc3ccc(Cl)c(Cl)c3)CC2)nc1 ; CN(C(=O)N1CCC(Nc2ncc(C(=O)NO)cn2)(c2ccccc2)CC1)c1ccccc1] 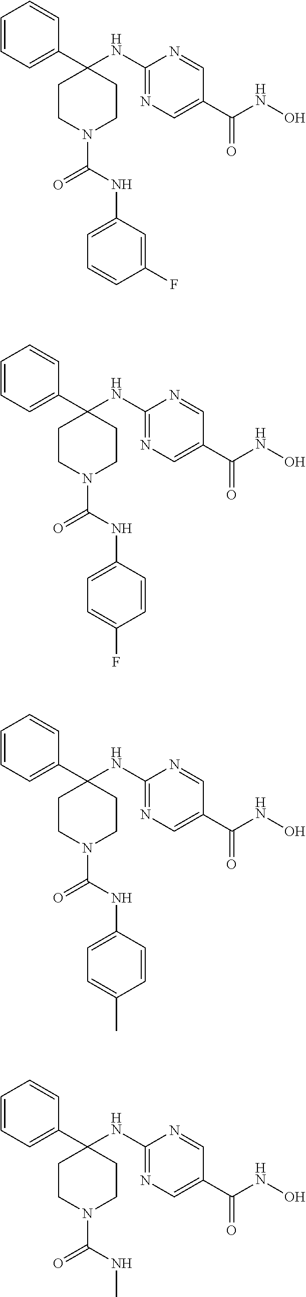 [SMILES: O=C(NO)c1cnc(NC2(c3ccccc3)CCN(C(=O)Nc3cccc(F)c3)CC2)nc1 ; O=C(NO)c1cnc(NC2(c3ccccc3)CCN(C(=O)Nc3ccc(F)cc3)CC2)nc1 ; Cc1ccc(NC(=O)N2CCC(Nc3ncc(C(=O)NO)cn3)(c3ccccc3)CC2)cc1 ; CNC(=O)N1CCC(Nc2ncc(C(=O)NO)cn2)(c2ccccc2)CC1] 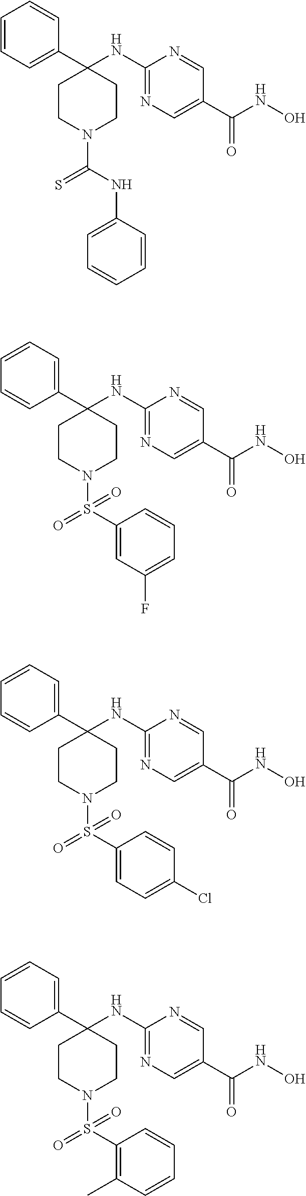 [SMILES: O=C(NO)c1cnc(NC2(c3ccccc3)CCN(C(=S)Nc3ccccc3)CC2)nc1 ; O=C(NO)c1cnc(NC2(c3ccccc3)CCN(S(=O)(=O)c3cccc(F)c3)CC2)nc1 ; O=C(NO)c1cnc(NC2(c3ccccc3)CCN(S(=O)(=O)c3ccc(Cl)cc3)CC2)nc1 ; Cc1ccccc1S(=O)(=O)N1CCC(Nc2ncc(C(=O)NO)cn2)(c2ccccc2)CC1] 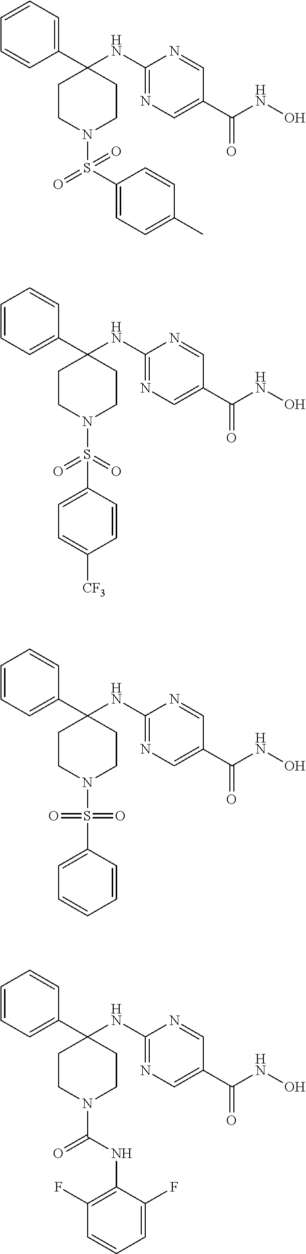 [SMILES: Cc1ccc(S(=O)(=O)N2CCC(Nc3ncc(C(=O)NO)cn3)(c3ccccc3)CC2)cc1 ; O=C(NO)c1cnc(NC2(c3ccccc3)CCN(S(=O)(=O)c3ccc(C(F)(F)F)cc3)CC2)nc1 ; O=C(NO)c1cnc(NC2(c3ccccc3)CCN(S(=O)(=O)c3ccccc3)CC2)nc1 ; O=C(NO)c1cnc(NC2(c3ccccc3)CCN(C(=O)Nc3c(F)cccc3F)CC2)nc1] 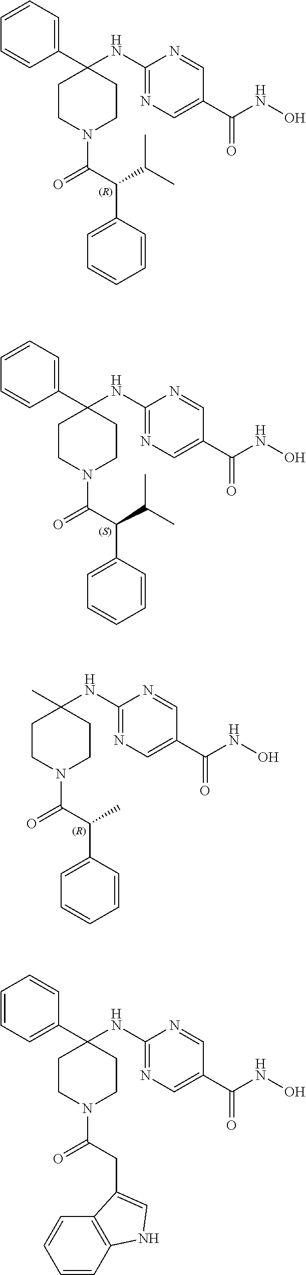 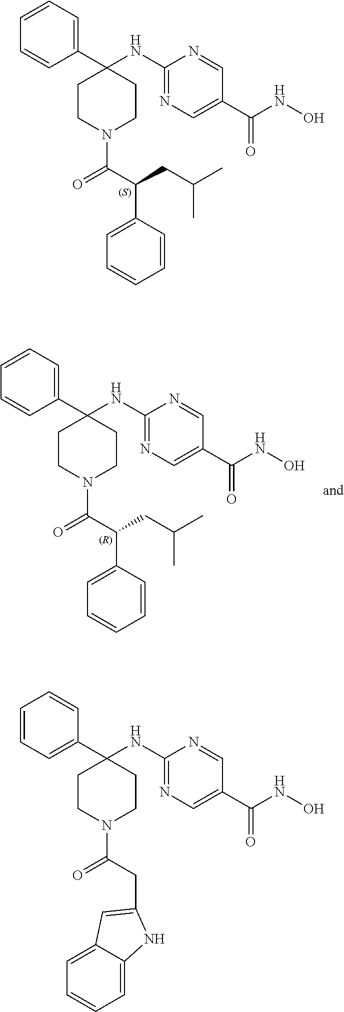
or a pharmaceutically acceptable salt thereof.

15. The method of claim 1, wherein the subject is a human.

16-32. (canceled)

33. A compound of Formula VII: 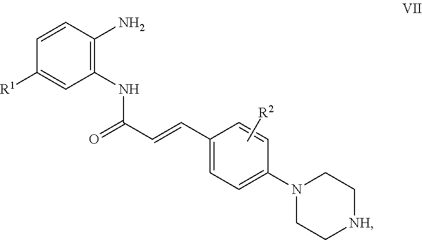
or a pharmaceutically acceptable salt thereof;
wherein
R¹ is phenyl or a 5-membered heteroaryl ring; and
R² is C₃₋₇-cycloalkyl.

34. A compound of Formula VIII: 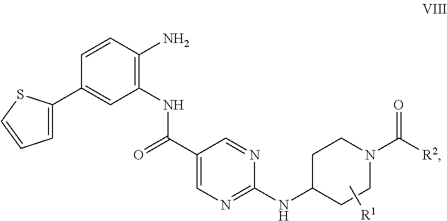
or a pharmaceutically acceptable salt thereof;
wherein
R¹ is C₁₋₄-alkyl; and
R² is a 5- or 6-membered heterocycloalkyl ring optionally substituted with C₁₋₄-alkyl.

35-41. (canceled)
